(19) United States
(12) Patent Application Publication
(10) Pub. No.: US 2015/0216409 A1
(43) Pub. Date: Aug. 6, 2015

(54) Methods And Apparatuses For Providing Laser Scanning Applications

(71) Applicant: Pro Fit Optix, Inc., Irving, TX (US)

(72) Inventors: William T. Hofmeister, Flower Mound, TX (US); Rishi Pampati, Frisco, TX (US); Jagdish Jethmalani, Irving, TX (US); Rudolf Suter, Dallas, TX (US)

(21) Appl. No.: 14/614,276

(22) Filed: Feb. 4, 2015

Related U.S. Application Data
(63) Continuation-in-part of application No. 14/469,351, filed on Aug. 26, 2014.
(60) Provisional application No. 61/936,219, filed on Feb. 5, 2014.
(60) Provisional application No. 61/970,640, filed on Mar. 26, 2014.
(60) Provisional application No. 61/970,640, filed on Mar. 26, 2014.

(51) Int. Cl. A61B 3/103; A61B 3/107

(52) U.S. Cl. CPC A61B 3/103; A61B 3/107

(57) ABSTRACT

An autorefractor-keratometer device including a first laser scanning device that emits a plurality of laser scans, a second laser device that emits a laser pulse, a first detector that detects the plurality of emitted laser line scans to determine corneal topography, and a second detector that detects a reflection of the emitted laser pulse to determine sphero-cylinder refraction.

### CROSS-REFERENCE TO RELATED APPLICATIONS

This application:
claims the priority, under 35 U.S.C. §119, of copending U.S. Provisional Patent Application No. 61/936,219, filed Feb. 5, 2014;
claims the priority, under 35 U.S.C. §119, of copending U.S. Provisional Patent Application No. 61/970,640, filed Mar. 26, 2014; and
is a continuation-in-part of U.S. patent application Ser. No. 14/469,351, filed Aug. 26, 2014 (which application claims the benefit under 35 U.S.C. §119, of copending U.S. Provisional Patent Application No. 61/970,640, filed Mar. 26, 2014);
   the entire disclosures of which are hereby incorporated by reference herein in their entirety.

### STATEMENT REGARDING FEDERALLY SPONSORED RESEARCH OR DEVELOPMENT

Not Applicable

### FIELD OF THE INVENTION

The present invention lies in the field of three-dimensional (3D) laser tracing using triangulation methodology. The present disclosure relates to systems and methods for 3D laser tracing of eyewear frames, lenses, lens template, groove, bevel, and drilled holes on lenses.

### BACKGROUND OF THE INVENTION

As shown in FIG. 1, the present process for manufacturing and delivering eyewear to customers or patients first involves customer contact with either a doctor's office, a storefront, or a store with its own manufacturing lab, which allows it to cut the lenses and fit them to the customer-selected frame. Most of the time, however, the customer interfaces with locations that do not have the ability to prepare their own lenses as well as to fit them to the frames selected by the customer. In such cases, the seller takes the selected frame and ships the frame along with the customer's eyeglass prescription to a global lens producer's manufacturing lab. The global lens producer manufactures the lenses and then sends the uncut lenses to the optical store/doctor. The optical store/doctor then cuts the lenses and fits them to the frames for final transfer to the customer.

FIG. 2 shows a possible alternative for such processing. Here, the seller takes the selected frame and ships the frame along with the customer's eyeglass prescription to a centralized glazing lab and frame warehouse. This warehouse collects and processes many such orders and sends them together to the global lens producer. The global lens producer manufactures the lenses and then sends the uncut lenses back with the box containing the prescription and the frames to the warehouse. The centralized lab cuts the lenses and sends the finished eyewear to the optical store/doctor. Either the centralized lab or the optical store/doctor fits the cut lenses to the frames. After fitting, the eyewear is transferred to the customer.

FIG. 3 is a map of the United States illustrating various centralized labs and manufacturers and the shipping routes between each. In either situation shown in FIGS. 1 and 2, not only is there a disadvantageous cost associated with sending the frames to a, typically, out-of-state lab, there also is a large percentage of breakage and loss while in transit. Once a frame actually arrives at the manufacturing lab, the frame is placed into a unique tray with the relevant paperwork. The tray is given to a lab technician for processing. First, the frame is scanned using a prior art mechanical frame tracer. Some of these tracers go under the names INDO S-Tracer, NIDEK tracer, BRIOT tracer, for example. These prior art mechanical frame tracers, however, have to be calibrated on regular basis, sometime daily and other times every three to four hours, and, if this is not done, the data that is output is not accurate. All output data from prior art frame tracers is input to a computer that converts the data to the Vision Counsel of America (VCA) data communication standard, which is the industry's standard for communicating frame tracing data. This data, which is created by the lab technician, is stored temporarily on the hard drive of the tracing unit. The lab technician automatically or manually transfers this scan data to a lab management system for processing as the lenses and frames are not typically created and fitted by the frame scanning lab technician. The generated data is stored on a shared location at the manufacturing lab, allowing all cutting technicians the ability to access this data. The generated data is also bound to the tray number corresponding to the tray in which the frames and paperwork are kept. The physical tray is then sent to one of the cutting technicians, who loads in the measured data based upon the tray identification number. This data and the tray are processed at a cutting station, at which station the cut lenses are also placed in the tray for assembly. The cutting technician or an assembly technician fits the lenses to the frames and the tray is sent to final processing, at which the assembled frames are, in theory, shipped to the location from which the frames were sent. Again, not only is there a disadvantageous cost associated with sending the frames back to the customer or to the customer's shop, there also is a large percentage of breakage and loss while in transit.

Just in the United States, many spectacle lens orders are submitted along with frames for manufacturing complete eyewear. During the twelve-month period from June 2012 to June 2013, for example, the Vision Council reported that 75 million completed prescription eyewear were sold in the U.S. The American Optometric Association reports that 26 million frames were shipped from an independent eyewear professional to a manufacturing lab. Revenue that is lost with these prior art processes includes the logistics cost of shipping the frames four times (consolidated shipping), the cost of lost or broken frames, and the cost of broken or scratched lenses. It is estimated that this cost is $40 million dollars per year.

Further, the prior art tracers are prone to errors, especially when the lab technicians fail to perform the required calibration at regular intervals. In such cases, the entire manufacturing, cutting, and fitting processes are repeated. Likewise, all of the shipping is repeated. This results in extensive increase of nonrecoverable costs.

In the ophthalmic industry, there are many processes that take place to generate prescription eyewear from thick semifinished lens blanks. These processes are performed on many different machines that block, generate prescription, polish, coat, and edge the lenses.

What is needed is an accurate and precise frame tracer that provides digital data to produce edged lenses for eyewear providers/stores to mount in frames. What is also needed is to reduce the amount and size of the different machines while also improving accuracy.

Thus, a need exists to overcome the problems with the prior art systems, designs, and processes as discussed above.

### SUMMARY OF THE INVENTION

The invention provides systems and methods of 3D laser tracing eyewear that overcome the hereinafore-mentioned disadvantages of the heretofore-known devices and methods of this general type and provide such features with a compact 3D laser frame tracer that is improved over the prior art mechanical frame tracers and that eliminate all need to ship a pair of frames to a lab, thereby eliminating the significant cost of shipment, loss, theft, and breakage.

The 3D laser tracer systems and processes are highly accurate and perform tracing of the frame without any contact. The systems and processes are capable of measuring frames with lenses or frames without lenses and of measuring just a single lens or lens template. The system is compact enough to fit on a user's desktop and, therefore, is placed easily in storefronts and doctors' offices, for example.

The systems and methods described herein are used as a recurring revenue generator. The systems and processes can be purchased or leased. After an initial installation fee, a transaction fee per frame can be charged as well as charging annual maintenance fees for normal wear and tear and software updates, for example. As the system is connected (wirelessly or wired) to the Internet, all use of the systems and processes can be monitored and all revenue generated can be tracked instantaneously. A cloud-based system (e.g., a server) can provide all data to both the manufacturer of the system and the users.

Various computer software is included in the systems and methods. First, laser detection and noise elimination algorithms are provided. Also, 3D reconstruction and registration functions are provided. User interface and system integration routines are included, as well as cloud integration programs.

Benefits provided by the disclosed systems and methods are many. First, frame measurement is more accurate. As such, more complete data is provided to reduce the need for re-cutting lenses. Customer satisfaction is improved because the lenses fit better and, therefore, there are less customer re-dos and returns. Due to its automatic functioning, the 3D laser tracing system requires little or no user interaction for scanning and requires little or no calibration. Further, all data provided into the system and used by the processes is shared across the cloud. The systems and methods also provide an accurate and precise Frame Trace Library that not only includes standard pre-defined frame trace measurements it also stores each frame measured in its own library. Therefore, when the same frame is measured more than once, the system is able to compare those measurements and, from this, determine characteristics of the frame, itself, such as manufacturing tolerances. With this, future measurements can be checked for additional accuracy. Overall, the systems and methods provide significant cost savings and improve customer satisfaction.

FIG. 4 directly compares the specifications of prior art mechanical frame tracers with an exemplary embodiment of a 3D laser tracer system described herein. Each of the measuring points, accuracy, and resolution are significantly improved over the prior art. Calibration is vastly superior. As for tracing time and scanning area, they are comparable. The system also provides free degree of rotation ranging from 0 degrees to up to 90 degrees for bevel/wrap/groove measurement.

FIG. 5 directly compares calibration, maintenance/wear, stylus instability, frame placement issues and measurement detail. Each are improved over the prior art.

The inventive 3D laser eyewear tracers and methods described herein obtain a significantly improved grade of data than prior art mechanical tracers. This occurs because the true shape of an eyewear frame only can be captured when the frame is undistorted from any pressure applied. When all prior art mechanical tracers attempt to measure an eyewear frame, pressure applied by the frame holding mechanism as well as the mechanical stylus, causes distortions in the shape of the lens opening. Therefore, subsequent tracing by the mechanical tracers measure a frame shape that is changed from the true shape. With the methods of frame tracing described herein, the non-contact scanning process along with the design of the frame holder eliminates any distortion caused by contact with the frames.

The calibration interval between the prior art and the inventive 3D laser eyewear tracers and methods described herein also is significant. All prior art mechanical tracers must be calibrated every time the machine is turned on. Also, if a prior art mechanical tracer is bumped while measuring or reaches a burr while measuring, then any measurement of that frame will be off from true and will not be detected by the lab. The lens cutter will only discover that error many steps down the process and then will have to request re-measurement of the eyewear, causing a significant delay in processing of that frame. Further, prior art manufacturers recommend re-calibration of the tracer every four hours of use. The inventive 3D laser eyewear tracers and methods described herein do not require such frequent calibration and are immune to such errors in measurement.

With the foregoing and other objects in view, there is provided, in accordance with the invention, an autorefractor-keratometer device including a first laser scanning device that emits a plurality of laser scans, a second laser device that emits a laser pulse, a first detector that detects the plurality of emitted laser line scans to determine corneal topography, and a second detector that detects a reflection of the emitted laser pulse to determine sphero-cylinder refraction.

Although the invention is illustrated and described herein as embodied in systems and methods for 3D laser frame tracing, it is, nevertheless, not intended to be limited to the details shown because various modifications and structural changes may be made therein without departing from the spirit of the invention and within the scope and range of equivalents of the claims. Additionally, well-known elements of exemplary embodiments of the invention will not be described in detail or will be omitted so as not to obscure the relevant details of the invention.

Additional advantages and other features characteristic of the present invention will be set forth in the detailed description that follows and may be apparent from the detailed description or may be learned by practice of exemplary embodiments of the invention. Still other advantages of the invention may be realized by any of the instrumentalities, methods, or combinations particularly pointed out in the claims.

Other features that are considered as characteristic for the invention are set forth in the appended claims. As required, detailed embodiments of the present invention are disclosed herein; however, it is to be understood that the disclosed embodiments are merely exemplary of the invention, which can be embodied in various forms. Therefore, specific structural and functional details disclosed herein are not to be interpreted as limiting, but merely as a basis for the claims and as a representative basis for teaching one of ordinary skill in the art to variously employ the present invention in virtually any appropriately detailed structure. Further, the terms and phrases used herein are not intended to be limiting; but rather, to provide an understandable description of the invention. While the specification concludes with claims defining the features of the invention that are regarded as novel, it is believed that the invention will be better understood from a consideration of the following description in conjunction with the drawing figures, in which like reference numerals are carried forward.

### BRIEF DESCRIPTION OF THE DRAWINGS

The accompanying figures, where like reference numerals refer to identical or functionally similar elements throughout the separate views, which are not true to scale, and which, together with the detailed description below, are incorporated in and form part of the specification, serve to illustrate further various embodiments and to explain various principles and advantages all in accordance with the present invention. Advantages of embodiments of the present invention will be apparent from the following detailed description of the exemplary embodiments thereof, which description should be considered in conjunction with the accompanying drawings in which:

FIG. 1 is a diagram of prior art global lens production;

FIG. 2 is a diagram of prior art global lens production with centralized glazing and frame warehousing;

FIG. 3 is a map of prior art shipment flow for lens manufacturing;

FIG. 4 is a chart comparing prior art mechanical lens tracer specifications with the 3D laser tracing system;

FIG. 5 is a chart comparing prior art mechanical lens tracer characteristics with the 3D laser tracing system;

FIG. 6 is an exploded, perspective view of an exemplary embodiment of a 3D laser eyewear tracer from above a front right corner;

FIG. 7 is a further exploded, perspective view of the 3D laser eyewear tracer of FIG. 6 with the display and eyewear tray removed;

FIG. 8 is an enlarged, perspective view of an exemplary embodiment of the eyewear tray of the 3D laser eyewear tracer of FIG. 6;

FIG. 9 is a diagram of an exemplary embodiment for processing laser-traced eyewear with the 3D laser eyewear tracer;

FIG. 10 is a perspective view of components of a 3D laser eyewear tracer tracing an eyewear lens with only the laser tracing visible, the cover/housing of the tracer removed;

FIG. 11 is a perspective view of the 3D laser eyewear tracer of FIG. 10 with the entire laser beam visible;

FIG. 12 is a perspective view of the components of the 3D laser eyewear tracer of FIG. 10 with a laser-triangulation polygon;

FIG. 13 is a perspective view of components of a 3D laser eyewear tracer tracing an eyewear frame having lenses;

FIG. 14 is a perspective view of the components of the 3D laser eyewear tracer of FIG. 13 with the entire laser beam visible;

FIG. 15 is a perspective view of an exemplary embodiment of a 3D laser eyewear tracer with the display closed from above a front right corner;

FIG. 16 is a perspective view of the 3D laser eyewear tracer of FIG. 15 with the display partially open;

FIG. 17 is a perspective view of the 3D laser eyewear tracer of FIG. 15 with the display open;

FIG. 18 is a perspective view of the 3D laser eyewear tracer of FIG. 17 with the outer shell transparent;

FIG. 19 is a perspective view of the 3D laser eyewear tracer of FIG. 17 with the outer shell removed;

FIG. 20 is a fragmentary, perspective view of the 3D laser eyewear tracer of FIG. 15 with an eyewear lens held in a lens-tracing position from above a left rear corner;

FIG. 21 is a fragmentary, perspective view of the 3D laser eyewear tracer of FIG. 15 with the eyewear lens held in a frame-tracing position from above a right rear corner;

FIG. 22 is an elevational view of another exemplary embodiment of a 3D laser eyewear tracer from a left side;

FIG. 23 is a perspective view of the 3D laser eyewear tracer of FIG. 22 from above a right front corner;

FIG. 24 is a perspective view of the 3D laser eyewear tracer of FIG. 23 with the front panel transparent;

FIG. 25 is a perspective view of the 3D laser eyewear tracer of FIG. 23 with the outer shell removed;

FIG. 26 is a perspective view of the 3D laser eyewear tracer of FIG. 26 from above a right rear corner;

FIG. 27 is a perspective view of the 3D laser eyewear tracer of FIG. 26 from above a left rear corner and with an eyewear lens held in a frame-tracing position;

FIG. 28 is a photograph of a perspective view of another exemplary embodiment of a 3D laser eyewear tracer from above a front left corner;

FIG. 29 is a photograph of a perspective view of the 3D laser eyewear tracer of FIG. 28 from above a rear left corner;

FIG. 30 is a photograph of a perspective view of the 3D laser eyewear tracer of FIG. 28 with a display in an open position and an eyewear drawer in a closed position;

FIG. 31 is a photograph of a perspective view of the 3D laser eyewear tracer of FIG. 30 with the eyewear drawer in an open position;

FIG. 32 is a perspective view of another exemplary embodiment of a 3D laser eyewear tracer from above a right from side and with the outer shell removed;

FIG. 33 is a perspective view of the 3D laser eyewear tracer of FIG. 32 with the display removed;

FIG. 34 is a perspective view of the 3D laser eyewear tracer of FIG. 32 from above a front right side with the scan compartment door in an open position;

FIG. 35 is a perspective view of the 3D laser eyewear tracer of FIG. 32 from above a front right side with the scan compartment door in a closed position;

FIG. 36 is a perspective view of the 3D laser eyewear tracer of FIG. 35 from above a left side with the outer shell removed;

FIG. 37 is a perspective view of the 3D laser eyewear tracer of FIG. 37 from above a front right corner with the scan compartment door in the open position;

FIG. 38A is a perspective view of a lens/frame holder of the 3D laser eyewear tracer of FIGS. 32 to 37 from above;

FIG. 38B is a side elevational view of the lens/frame holder of the 3D laser eyewear tracer of FIG. 38A;

FIG. 38C is a side elevational view of the lens/frame holder of the 3D laser eyewear tracer of FIG. 38B rotated 80 degrees for scanning the bevel of the eyeglass frame;

FIG. 38D is a perspective view of a frame mounted on the holder of the 3D laser eyewear tracer of FIGS. 32 to 38C with a diagrammatic representation of the camera's field of view;

FIG. 38E is a top plan view of the frame mounted on the holder of the 3D laser eyewear tracer of FIG. 38D with the diagrammatic representation of the camera's depth of field of view;

FIG. 38F is a top plan view of the frame and holder of the 3D laser eyewear tracer of FIG. 38E with the frame rotated with respect to the laser and camera;

FIG. 38G is a perspective view of the frame and holder of the 3D laser eyewear tracer from a side thereof with a diagrammatic representation of the camera's diameter of field of view;

FIG. 38H is a front side elevational view of the frame and holder of the 3D laser eyewear tracer of FIG. 38G;

FIG. 38I is a perspective view of a lens holder attachment for mounting a lens or lens template to the lens/frame holder of FIG. 38A;

FIG. 38J is a perspective view of the lens holder attachment of FIG. 38I inserted into a portion of the lens/frame holder of FIG. 38A;

FIG. 39 is a process flow diagram of an exemplary embodiment for tracing eyewear with the 3D laser eyewear tracer;

FIG. 40 is a diagram of an exemplary embodiment of a user interface for the 3D laser eyewear tracer;

FIG. 41 is a diagram of the user interface of FIG. 40 with one eyewear lens traced;

FIG. 42 is a diagram of the user interface of FIG. 40 with both eyewear lenses traced;

FIG. 43 is a diagram of an exemplary embodiment of a user interface for drill processing a left lens with the 3D laser eyewear tracer;

FIG. 44 is a diagram of an exemplary embodiment of a user interface for drill processing a right lens with the 3D laser eyewear tracer;

FIG. 45 is a diagram of an exemplary embodiment of a user interface for drill frame trace placement with the 3D laser eyewear tracer;

FIG. 46 is a diagram of the user interface of FIG. 45 with one eyewear lens traced;

FIG. 47 is a diagram of the user interface of FIG. 45 with two eyewear lenses traced;

FIG. 47 is a diagram of the user interface of FIG. 45 with both eyewear lenses traced;

FIG. 48 is a fragmentary computer code listing of a lens tracing with the 3D laser eyewear tracer in the VCA format;

FIGS. 49 to 73 are periodic photographs of a laser tracing operation of an eyewear frame having lenses with the 3D laser eyewear tracer;

FIGS. 74 to 91 are periodic photographs of a laser tracing operation of a single eyewear lens with the 3D laser eyewear tracer;

FIGS. 92 to 97 are front views of a captured laser tracing of an eyewear frame in various processing steps from the camera capture to the edge processing;

FIG. 97A is a front view of a captured laser tracing of an eyewear frame that is divided into four quadrants, each of which possessing different Z values;

FIG. 97B is a front view of a captured laser tracing of an eyewear frame for one lens of the frame of FIG. 97A and which is further divided into two quadrants, each of which possessing different Z values;

FIG. 98 is a front view of all possible frame coordinates for another eyewear frame where the right lens is removed and the left lens is still mounted in the frame;

FIG. 99 is a front view of all possible frame edges for the eyewear frame of FIG. 98 where the frame edges are detected in the process of FIGS. 92 to 97;

FIG. 100 is an outer edge of a top of a right half of the frame of FIG. 98 without the lens;

FIG. 101 is an inner edge of the top of the right half of the frame of FIG. 98 without the lens;

FIG. 102 is an inner edge of a bottom of the right half of the frame of FIG. 98 without the lens;

FIG. 103 is an outer edge of the bottom of the right half of the frame of FIG. 98 without the lens;

FIG. 104 is the complete edge detail of the right half of the frame of FIG. 98 without the lens;

FIG. 105 is the complete lens edge of the right half of the frame of FIG. 98 without the lens;

FIG. 106 is a refined lens edge of the right half of the frame of FIG. 98 without the lens;

FIG. 107 is a depiction of a final step in a process for determining the lens edge of the right half of the frame of FIG. 98 without the lens;

FIG. 108 is a flow chart of an exemplary embodiment of an edge sorting algorithm according to the invention;

FIG. 109 is a 3D contour of a metal eyeglass frame from a tracer and which shows a wrap in the frame design;

FIG. 110 is a 3D scan of an inside bevel of a plastic frame laser traced by the 3D laser eyewear tracer;

FIG. 111 is a 3D scan of a frame bevel laser traced by the 3D laser eyewear tracer;

FIG. 112 is a dissection of the 3D scan of the frame bevel of FIG. 111;

FIG. 113 is a depiction of a dissected contour of the 3D scan of the frame bevel of FIG. 111 which shows that the bevel is shaped as U or V;

FIG. 114 is a 3D scan of a lens bevel laser traced by the 3D laser eyewear tracer;

FIG. 115 is a dissection of the 3D scan of the lens bevel of FIG. 114;

FIG. 116 is a depiction of a dissected contour of the 3D scan of the lens bevel of FIG. 114 which shows that the bevel is shaped as U or V;

FIG. 117 is a 3D scan of a groove portion on an edge of a lens laser traced by the 3D laser eyewear tracer;

FIG. 118 is a dissection of the 3D scan of the groove portion of the lens of FIG. 117;

FIG. 119 is a depiction of a dissected contour of the groove portion of the lens of FIG. 117 which is shaped as a trench;

FIG. 120 is a raw scan of a single lens along with laser scattering by the 3D laser eyewear tracer;

FIG. 121 is an edge detection of the single lens of FIG. 119 by the 3D laser eyewear tracer;

FIG. 122 is an unprocessed edge profile of the single lens of FIG. 120 by the 3D laser eyewear tracer after removal of laser scattering and noise with roughness in the edge profile;

FIG. 123 is a processed edge profile of the single lens of FIG. 120 with a smoothing function applied by the 3D laser eyewear tracer to create a smooth edge;

FIG. 124 is a comparison of lens traces from a prior art mechanical tracer and the 3D laser eyewear tracer with an overlay of the two disposed therebetween;

FIG. 125 is a diagram of the inventive process that permits sending only the prescription along with an accurate frame trace to a global lens production lab and the lab sending finished eyewear or edge lenses that could be mounted in frame to a storefront without shipment of frames;

FIG. 126 is a diagram of cloud-based communication between the 3D laser eyewear tracer and a cloud file server through the internet cloud from which frame trace data can be remotely accessed by a handheld device, a terminal, or a computer;

FIG. 127 is a diagram of details of cloud-based data sharing between the 3D laser eyewear tracer and smart devices, terminals, optical labs, and other web resources that transfer trace data;

FIG. 128 is a block diagram of possible connections between the 3D laser eyewear tracer (aka, the SmartTracer) from an eye care practitioner office through practice management software to a lab via a cloud-based data sharing using lab management software and communication with various different edger types at the lab or at the eye care practitioner office

FIG. 129 is a diagram illustrating applications of the inventive laser scanning system;

FIG. 130 is a chart comparing prior art blocker solutions with the 3D laser scanning system;

FIG. 131 is a chart comparing prior art blocker specifications with the 3D laser scanning system;

FIG. 132 is a chart comparing prior art lens mapper solutions with the 3D laser scanning system;

FIG. 133 is a chart comparing prior art lens mapper specifications with the 3D laser scanning system;

FIG. 134 is a chart comparing prior art lens mapper/finish blocker solutions with the 3D laser scanning system;

FIG. 135 is a chart comparing prior art lens mapper/finish blocker specifications with the 3D laser scanning system;

FIG. 136 illustrates a prior art corneal topographer;

FIGS. 137 to 150 are periodic diagrams of a laser scanning operation of a cornea;

FIG. 151 is a chart comparing prior art placido disc solutions with the 3D laser scanning system;

FIG. 152 is a chart comparing prior art placido disc solution specifications with the 3D laser scanning system;

FIG. 153 is a prior art Autorefractor;

FIGS. 154 to 167 are periodic diagrams of a laser scanning operation of a cornea;

FIGS. 168 to 174 are periodic diagrams of a refraction measurement and pupil diameter measurement using a laser pulse;

FIG. 175 is a chart comparing prior art placido disc solutions with the laser scanning system;

FIG. 176 is a chart comparing prior art placido disc solution specifications with the laser scanning system;

FIG. 177 is a perspective view of a laser scanner with a frame mounted on the holder of the laser scanner with a diagrammatic representation of a camera's depth of field of view where the camera is orthogonal and a laser is placed at an angle;

FIG. 178 is a perspective view of the laser scanner of FIG. 177;

FIG. 179 is a top plan view of the laser scanner of FIG. 177; and

FIG. 180 is a graphical representation of an exemplary embodiment of neighbor counting.

### DETAILED DESCRIPTION OF THE INVENTION

As required, detailed embodiments of the present invention are disclosed herein; however, it is to be understood that the disclosed embodiments are merely exemplary of the invention, which can be embodied in various forms. Therefore, specific structural and functional details disclosed herein are not to be interpreted as limiting, but merely as a basis for the claims and as a representative basis for teaching one skilled in the art to variously employ the present invention in virtually any appropriately detailed structure. Further, the terms and phrases used herein are not intended to be limiting; but rather, to provide an understandable description of the invention. While the specification concludes with claims defining the features of the invention that are regarded as novel, it is believed that the invention will be better understood from a consideration of the following description in conjunction with the drawing figures, in which like reference numerals are carried forward.

Alternate embodiments may be devised without departing from the spirit or the scope of the invention. Additionally, well-known elements of exemplary embodiments of the invention will not be described in detail or will be omitted so as not to obscure the relevant details of the invention.

Before the present invention is disclosed and described, it is to be understood that the terminology used herein is for the purpose of describing particular embodiments only and is not intended to be limiting. The terms “a” or “an”, as used herein, are defined as one or more than one. The term “plurality,” as used herein, is defined as two or more than two. The term “another,” as used herein, is defined as at least a second or more. The terms “including” and/or “having,” as used herein, are defined as comprising (i.e., open language). The term “coupled,” as used herein, is defined as connected, although not necessarily directly, and not necessarily mechanically.

Relational terms such as first and second, top and bottom, and the like may be used solely to distinguish one entity or action from another entity or action without necessarily requiring or implying any actual such relationship or order between such entities or actions. The terms “comprises,” “comprising,” or any other variation thereof are intended to cover a non-exclusive inclusion, such that a process, method, article, or apparatus that comprises a list of elements does not include only those elements but may include other elements not expressly listed or inherent to such process, method, article, or apparatus. An element proceeded by “comprises . . . a” does not, without more constraints, preclude the existence of additional identical elements in the process, method, article, or apparatus that comprises the element.

As used herein, the term “about” or “approximately” applies to all numeric values, whether or not explicitly indicated. These terms generally refer to a range of numbers that one of skill in the art would consider equivalent to the recited values (i.e., having the same function or result). In many instances these terms may include numbers that are rounded to the nearest significant figure.

It will be appreciated that embodiments of the invention described herein may be comprised of one or more conventional processors and unique stored program instructions that control the one or more processors to implement, in conjunction with certain non-processor circuits and other elements, some, most, or all of the functions of the powered injector devices described herein. The non-processor circuits may include, but are not limited to, signal drivers, clock circuits, power source circuits, and user input and output elements. Alternatively, some or all functions could be implemented by a state machine that has no stored program instructions, or in one or more application specific integrated circuits (ASICs) or field-programmable gate arrays (FPGA), in which each function or some combinations of certain of the functions are implemented as custom logic. Of course, a combination of these approaches could also be used. Thus, methods and means for these functions have been described herein.

The terms “program,” “software,” “software application,” and the like as used herein, are defined as a sequence of instructions designed for execution on a computer system. A “program,” “software,” “application,” “computer program,” or “software application” may include a subroutine, a function, a procedure, an object method, an object implementation, an executable application, an applet, a servlet, a source code, an object code, a shared library/dynamic load library and/or other sequence of instructions designed for execution on a computer system. The term “scanning” or “tracing” and the like as used herein, are defined as a 3D laser scan of eyeglass frames or lenses or lens templates or bevel or groove.

Herein various embodiments of the present invention are described. In many of the different embodiments, features are similar. Therefore, to avoid redundancy, repetitive description of these similar features may not be made in some circumstances. It shall be understood, however, that description of a first-appearing feature applies to the later described similar feature and each respective description, therefore, is to be incorporated therein without such repetition.

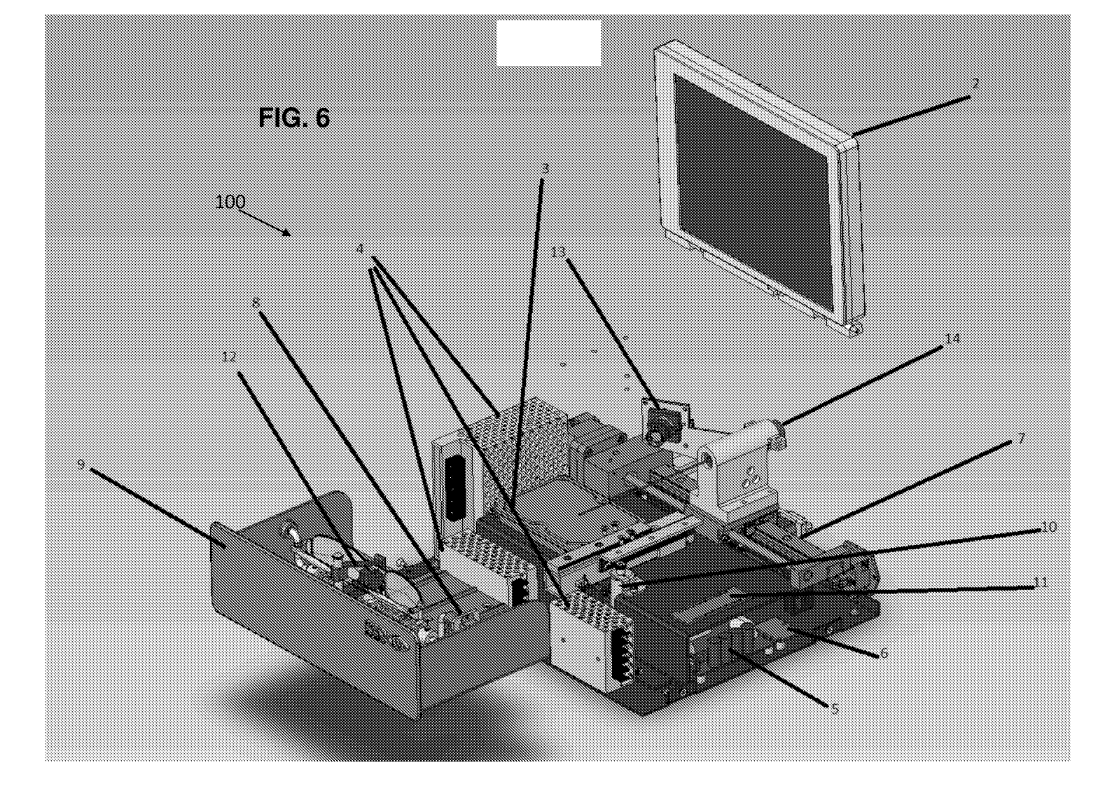

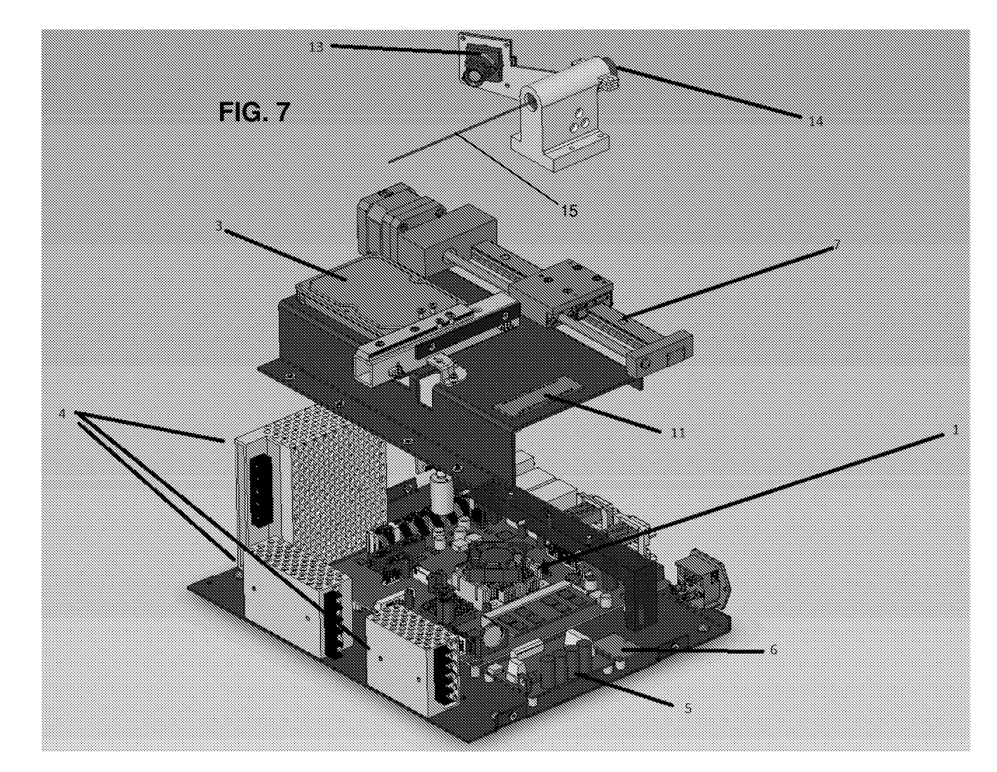

Described now are exemplary embodiments of the present invention. Referring now to the figures of the drawings in detail and first, particularly to FIGS. 6 to 8, there is shown a first exemplary embodiment of a 3D laser eyewear tracer 100 having an embedded computer 1 that provides control of all mechanisms and performs processing of all data, for example, the computer 1 can be a Windows-based single board computer. The computer 1 provides control of all mechanisms and performs processing of all data. The computer 1 also facilitates communication with external systems via Ethernet, Wi-Fi, USB and RS232, to name a few. A display 2, e.g., a touch screen, provides the viewable data and user interfaces for the program that executes the laser tracing. The display 2 is connected to the computer 1, for example, through a DVI or USB connection. A storage unit 3, e.g., a hard drive or other memory, provides local storage of all programs and data for the computer 1. One or more power supplies 4 provide power for the computer 1 as well as all drive motors/controllers, cameras 13, and lasers 14. A stepper motor controller 5 provides control of direction and speed of a stepper motor within a linear drive mechanism 7. The computer 1 communicates to the stepper motor controller 5 through, for example, a USB interface. A servo motor controller 6 provides control of direction and speed of a servomotor within a rotary drive mechanism 8. The computer 1 communicates to the servo motor controller 6 through, for example, a USB interface. The linear drive mechanism 7 facilitates lateral scan motion of the camera/laser assembly 13, 14. A stepper motor drives the linear drive mechanism 7. The rotary motion drive mechanism 8 facilitates rotation of the frame/lens so that scans can be performed at specific and/or varied angles.

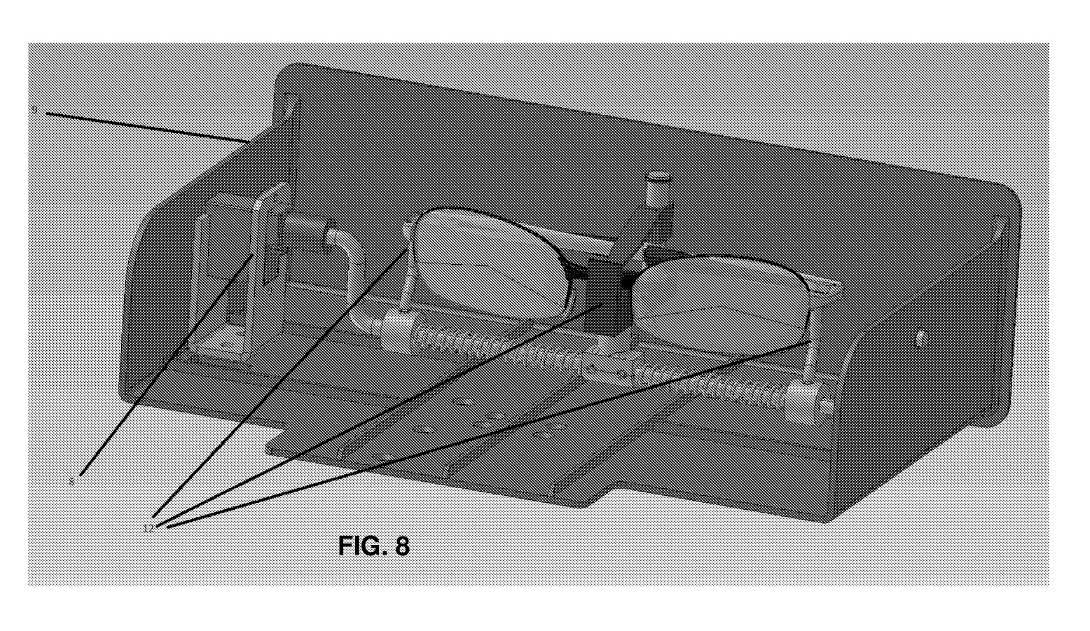

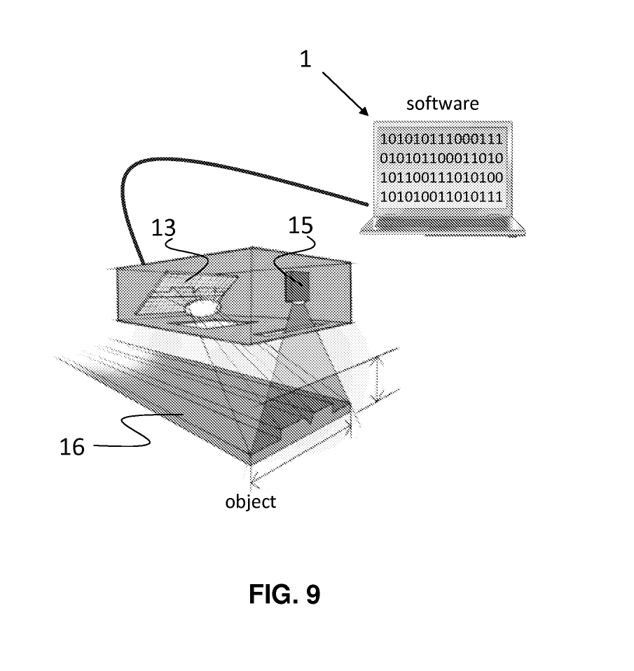

A slide tray 9 houses the rotary motion drive mechanism 8 and a frame/lens holder 12. The slide tray 9 is able to slide into and out from the non-illustrated housing of the tracer 100 to allow a user to place the frame/lens into the frame/lens holder 12 for scanning. A slide tray motor 10 facilitates motion of the slide tray 9. A slide tray motor driver 11 controls speed and direction of the slide tray motor 10 and is controlled, for example, through a digital I/O from the computer 1. The frame/lens holder 12 is shown in FIGS. 6 to 8 as holding the frame/lens in a stable position that is perpendicular to the camera/laser assembly 13, 14. In this exemplary embodiment, the frame/lens holder 12 of the tracer 100 can rotate the frame/lens when desired. In an alternative embodiment, the frame/lens holder 12 fixedly holds the frame/lens in a given position, e.g., substantially perpendicular to a left-right translation assembly (e.g., the linear drive mechanism 7), and it is the camera/laser assembly 13, 14 that not only translates from left to right with respect to the frame/lens but also pivots/rotates about the frame/lens. In such a configuration, the frame/lens experiences no movement and, therefore, scanning errors that could arise from frame/lens movement (e.g., bouncing) are eliminated. The camera 13 is attached to the linear drive mechanism 7 and captures images of a laser line from the laser 14 as the laser 14 moves across the object being scanned. The computer 1 communicates to the camera 13, for example, through a USB connection. The laser 14 also is attached to the linear drive mechanism 7 and generates a laser line 15 (or a laser path such as shown in FIGS. 9, 11, 12, and 14) that is scanned laterally across the surface of the object being scanned as the linear drive mechanism 7 translates, for example, from left to right or right to left.

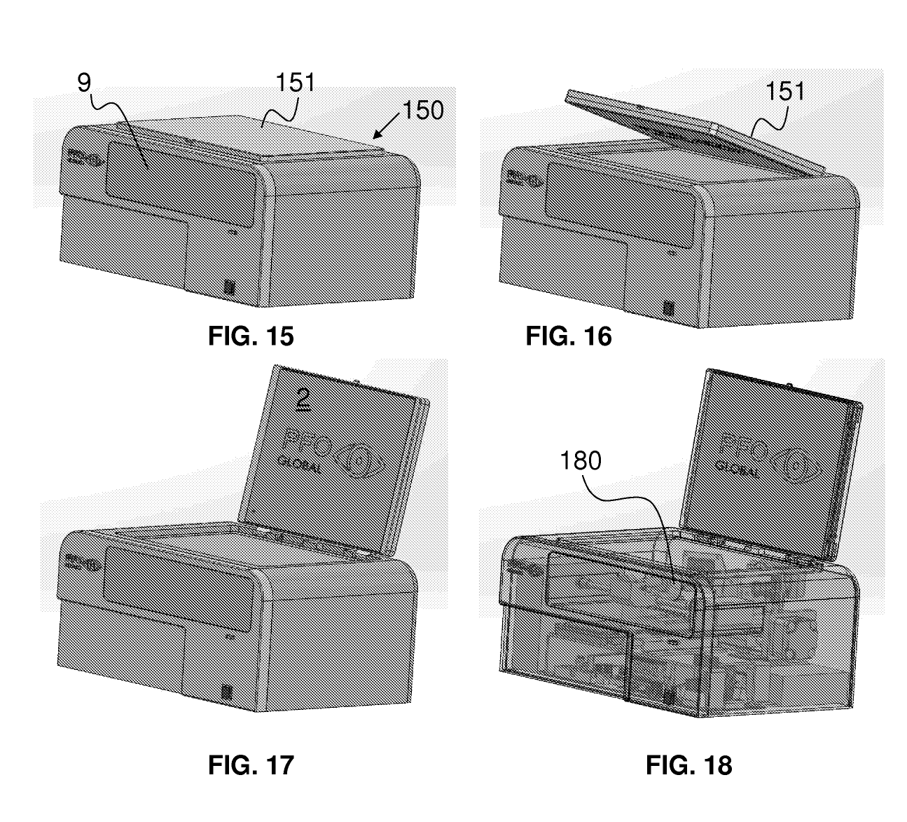

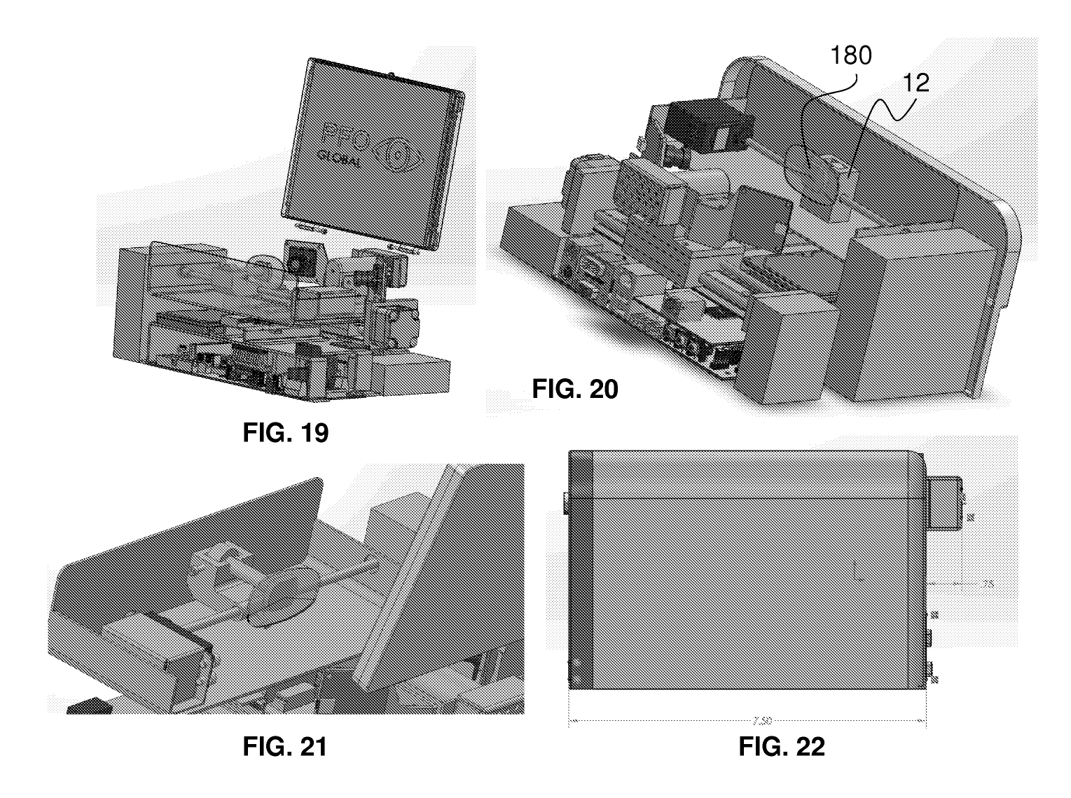

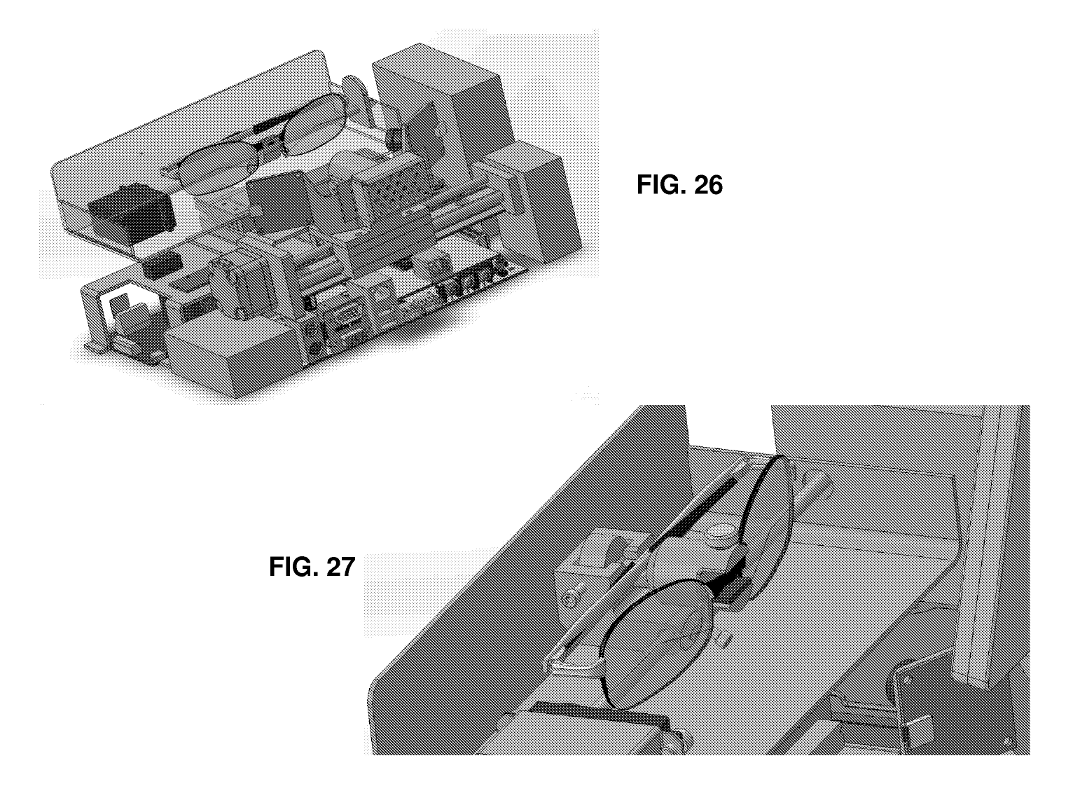

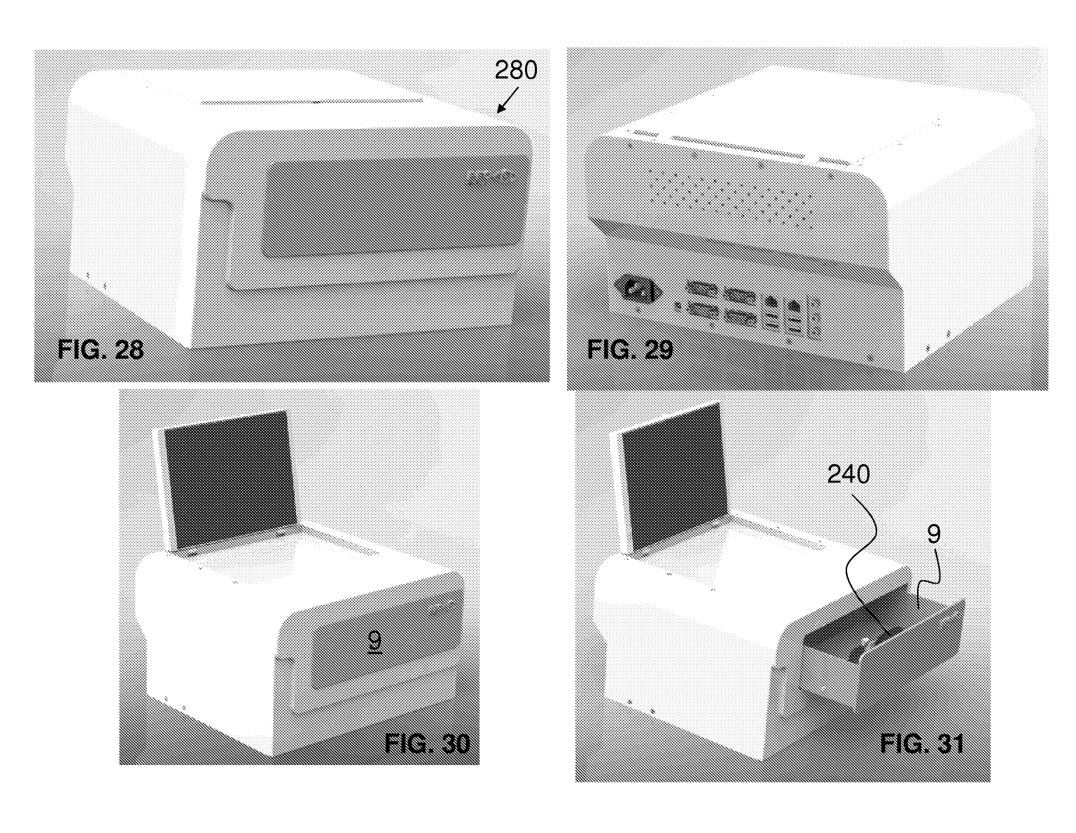

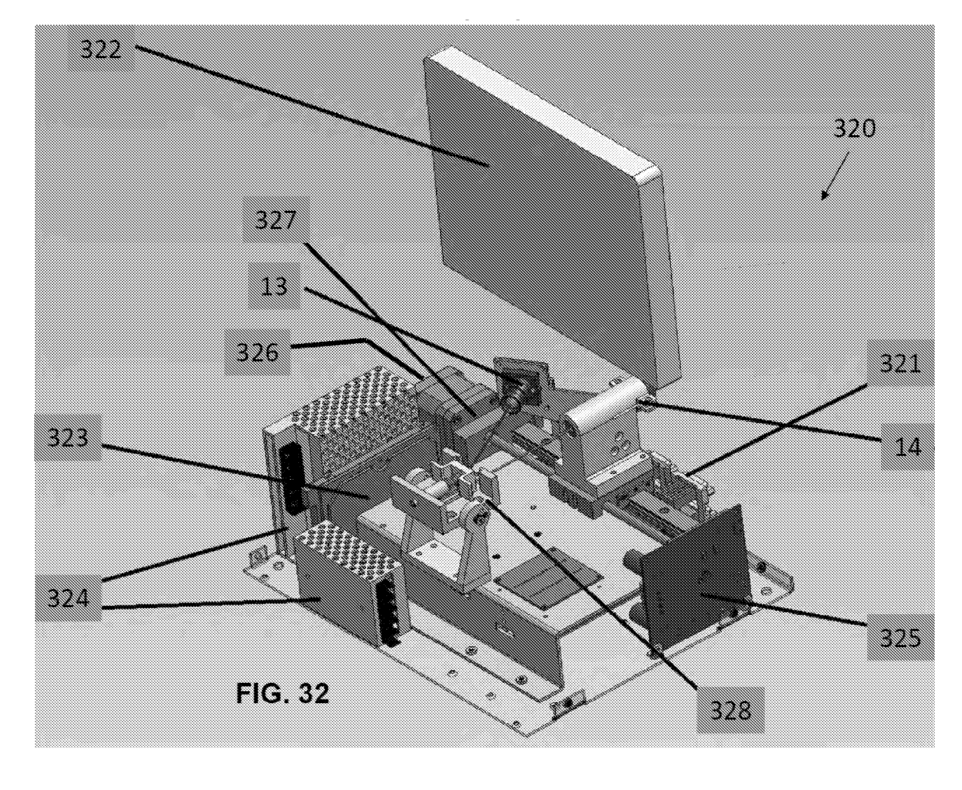

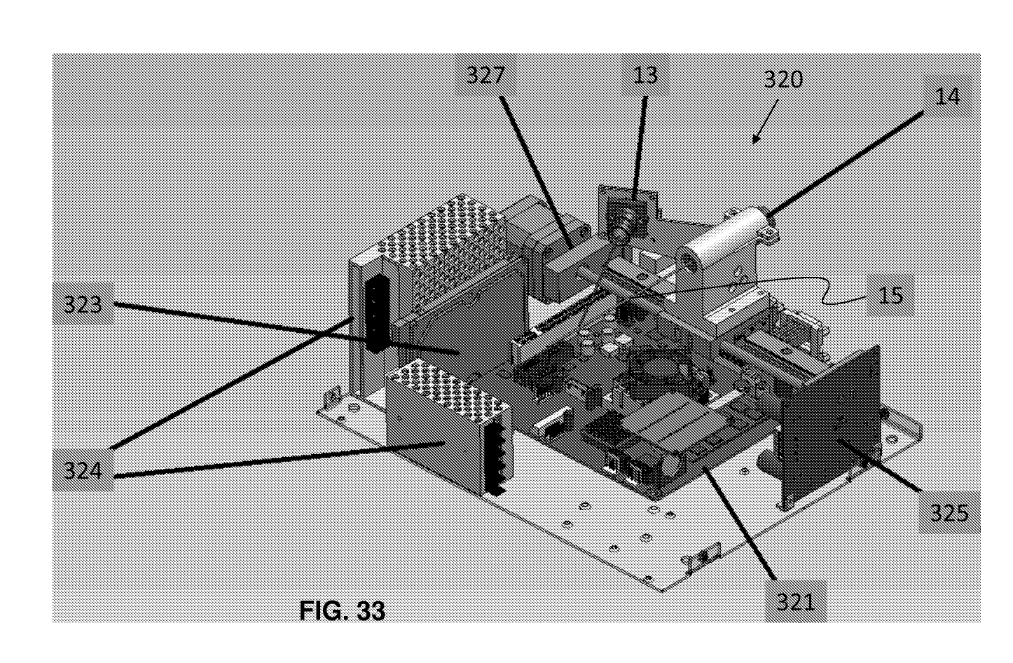

FIGS. 15 to 31 illustrate exemplary embodiments of finished 3D laser tracer products including exemplary embodiments of a 3D laser tracer. FIGS. 15 to 22 illustrate a 3D laser tracer product 150 measuring a single lens 180. In FIG. 15, the 3D laser tracer product 150 is closed and ready to use. The top 151 is opened revealing a touch-screen display 2 protected inside. With the top 151 open and the display 2 facing the user, the 3D laser tracer product 150 is ready for laser tracer scanning. The slide tray 9 is opened and a single lens 180 is held in the lens holder 12 for scanning. FIG. 18 illustrates the body of the 3D laser tracer product 150 transparent to reveal the lens 180 held in the lens holder 12 for scanning. In FIGS. 19 to 21, the outer body of the 3D laser tracer product 150 is removed, revealing the lens 180 in position for scanning.

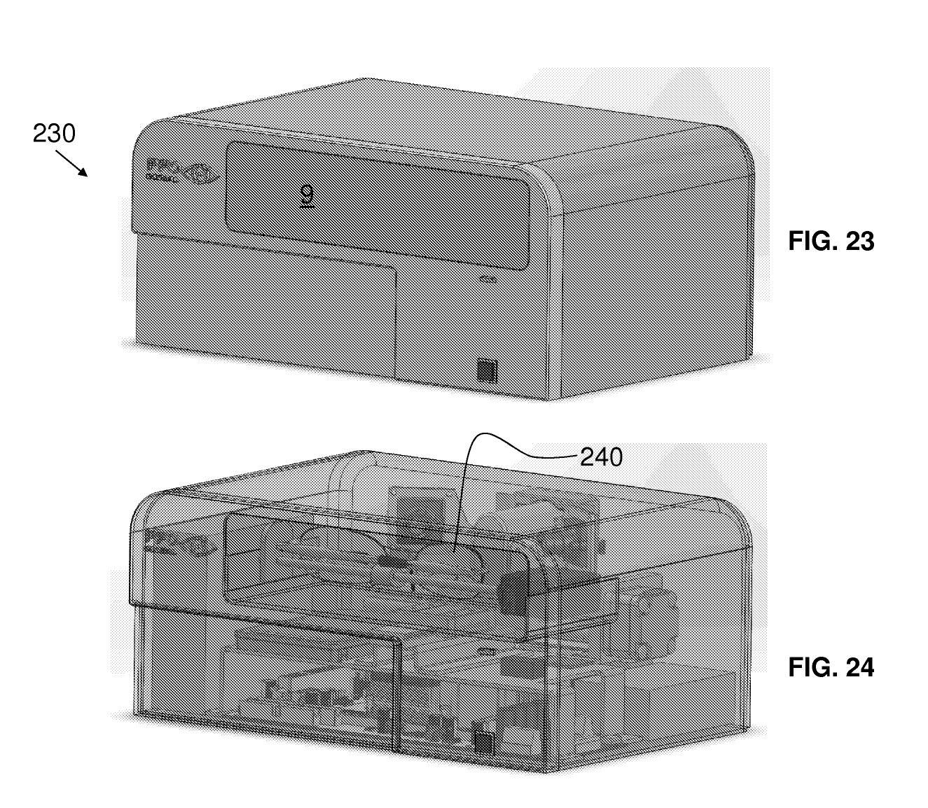

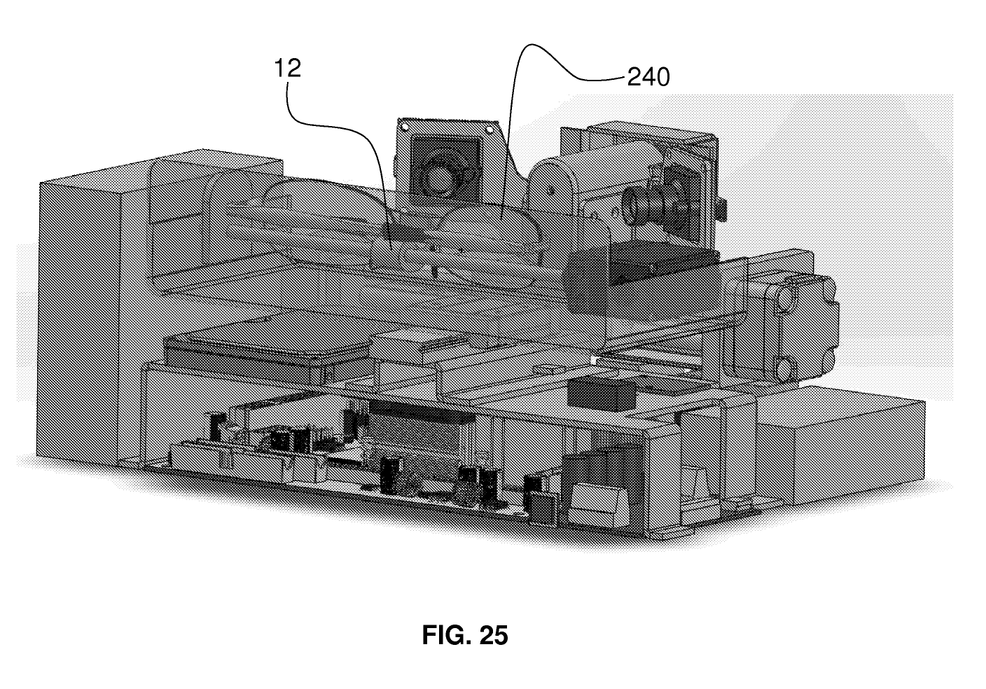

FIGS. 23 to 32 illustrate a 3D laser tracer product measuring an eyewear frame. In FIG. 23, the 3D laser tracer product 230 is closed and ready to use. The slide tray 9 is opened and an eyewear frame 240 is held in the frame holder 12 for scanning. FIGS. 25 to 27 illustrate the body of the 3D laser tracer product 230 removed to reveal the frame 240 held in the frame holder 12 for scanning.

FIGS. 28 to 31 also illustrate a 3D laser tracer product 280 measuring an eyewear frame. In FIGS. 28 and 29, the 3D laser tracer product 280 is closed and ready to use. The slide tray 9 is moved to the open position in the transition from FIG. 30 to FIG. 31 and an eyewear frame 240 is placed in the frame holder 12 for scanning.

Referring now to the figures of the drawings in detail and, particularly, to FIGS. 32 to 38J, there is shown another exemplary embodiment of a 3D laser eyewear tracer 320 having an embedded computer 321 that provides control of all mechanisms and performs processing of all data, for example, the computer 321 can be a Windows-based single board computer. The computer 321 provides control of all mechanisms and performs processing of all data. The computer 321 also facilitates communication with external systems via Ethernet, Wi-Fi, USB and RS232, to name a few. A display 322, e.g., a touch screen, provides the viewable data and user interfaces for the program that executes the laser tracing. The display 322 is connected to the computer 321, for example, through a DVI or USB connection. A storage unit 323, e.g., a hard drive or other memory, provides local storage of all programs and data for the computer 321. One or more power supplies 324 provide power for the computer 321 as well as all drive motors/controllers, cameras 13, and lasers 14. A stepper motor controller 325 provides control of direction and speed of a stepper motor 326 within a linear drive mechanism 327. The computer 321 communicates to the stepper motor controller 325 through, for example, a USB interface. The linear drive mechanism 327 facilitates lateral scan motion of the camera/laser assembly 13, 14 while the stepper motor 326 drives the linear drive mechanism 327. The camera 13 captures the images of the laser line as it moves across the object being scanned. The embedded computer 321 communicates to the camera 13 via USB. A frame holder mechanism 328 facilitates rotation of the frame/lens so that scans can be performed at specific and/or varied angles.

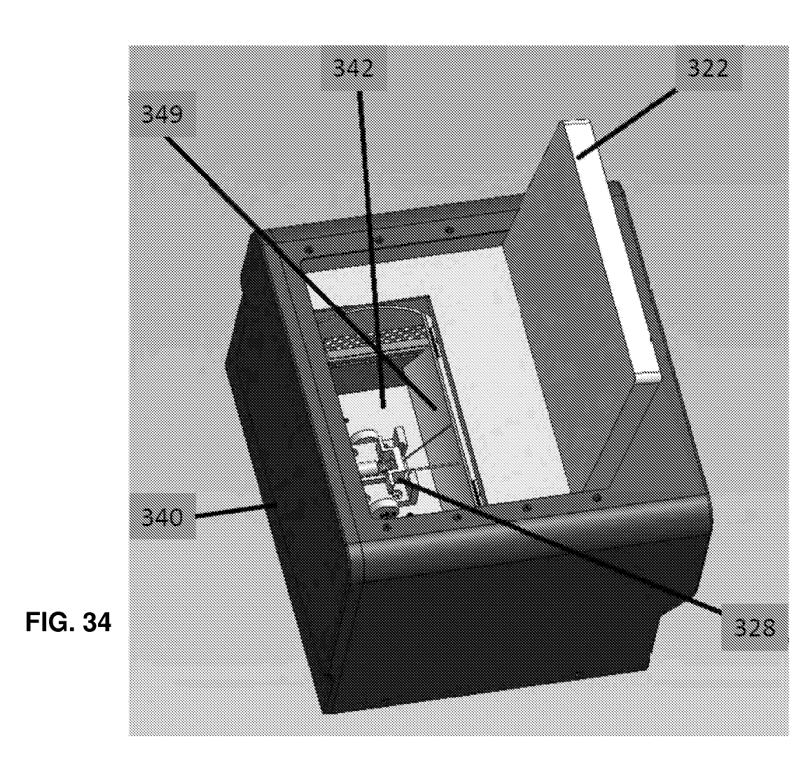

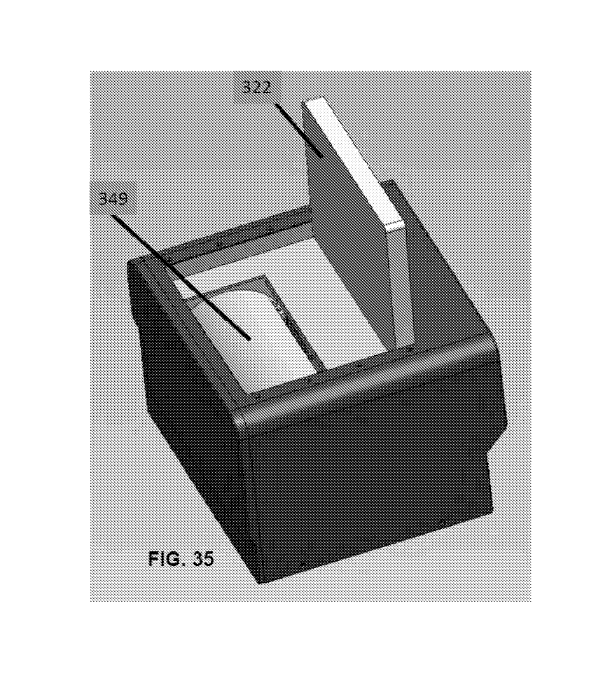

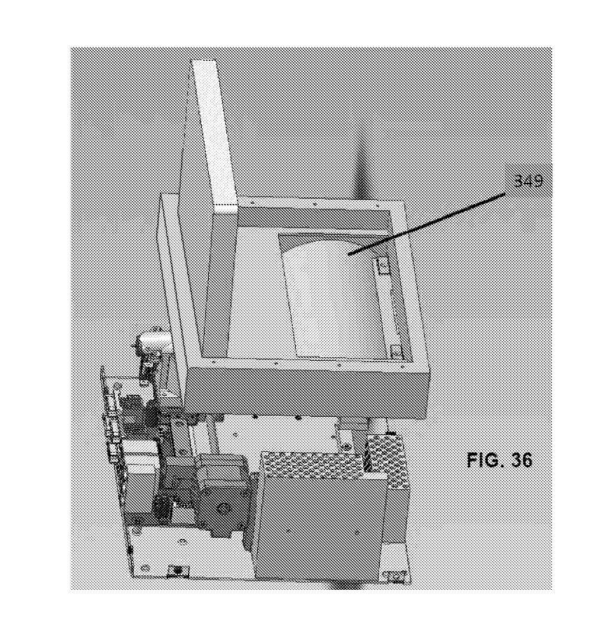

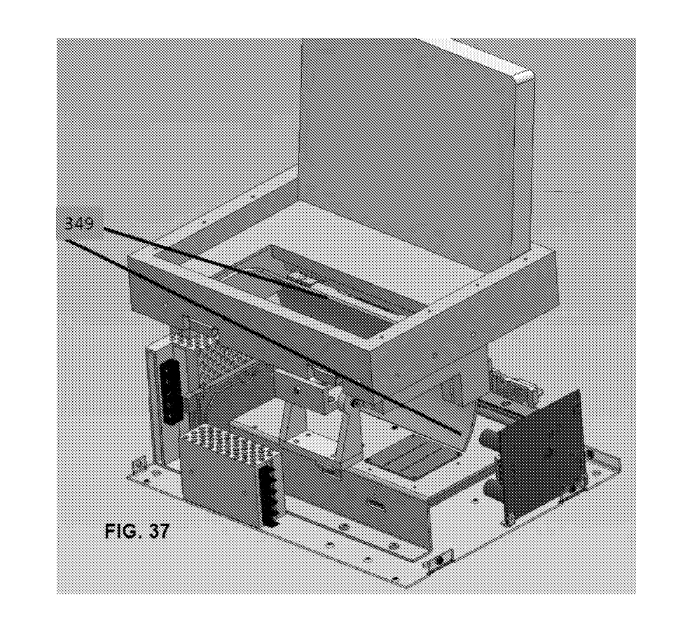

In this exemplary embodiment, instead of a slide tray, an outer case 340 (shown in FIGS. 34 and 35) houses all of the above components (as shown in FIGS. 36 to 38J in which the outer case 340 is removed) and defines therein a scan compartment 342. The scan compartment 342 is an enclosed area, located in the top of the unit, in which the frames and lenses are scanned. The frame/lens holder 328 is located in this area. There is also a scan compartment door 349, located on the top of the scan compartment 342, which, when open, allows an operator to place the frames/lenses into the frame/lens holder 328. The scan compartment door 349 closes off the scan compartment 342 from the environment outside the outer case 340, as shown in FIG. 35. In this embodiment, the scan compartment door 349 is made of a tinted plastic, which allows only a limited amount of light to enter or exit the scan compartment 342 when the door 349 is closed. The tinted door 349 allows the operator to observe the scan progress, but does not allow enough ambient light to interfere with the scan process. If desired, a safety interlock is associated with the door 349 so that the laser 14 cannot be activated while the door 349 is open. The door 349 is configured to block the user's view of all internal components when the door 349 is open. Magnetic catches are utilized to assist in keeping the door 349 in either the open or closed positions.

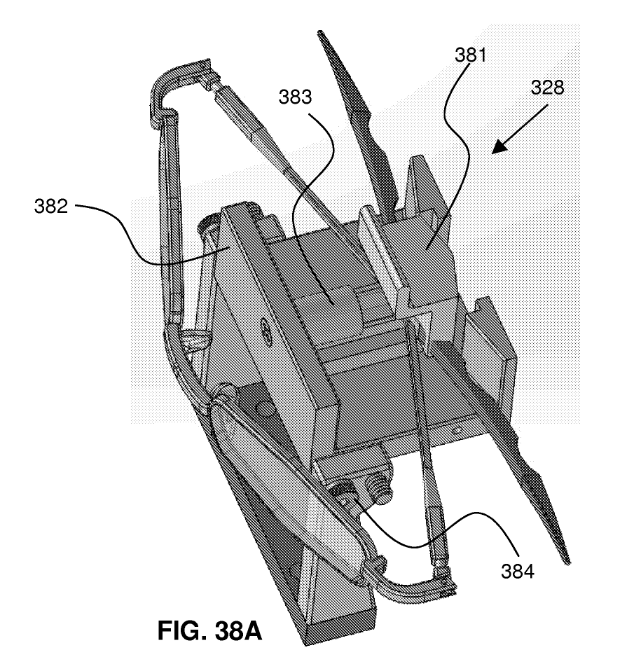

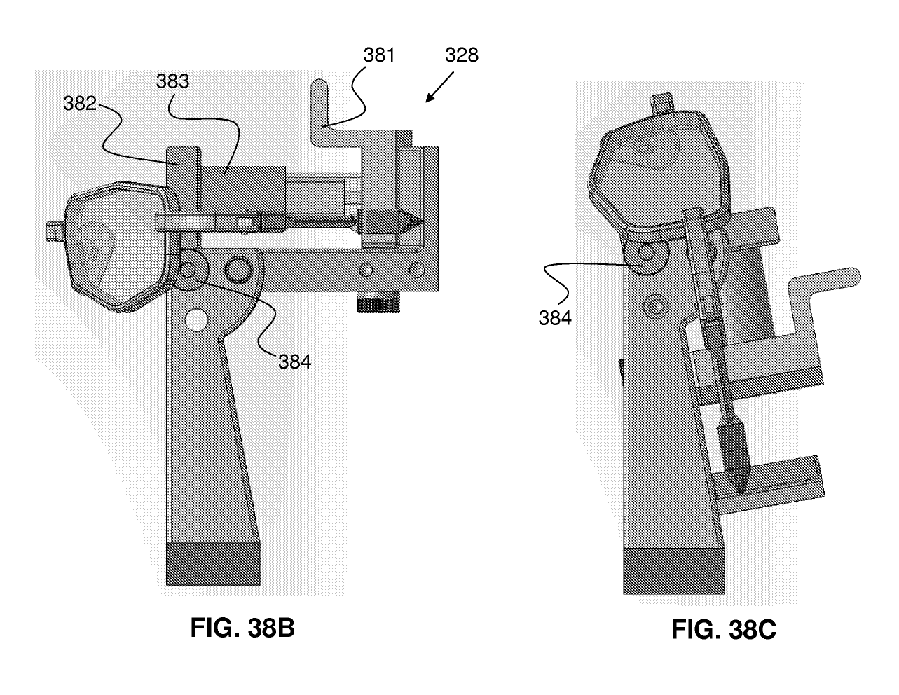

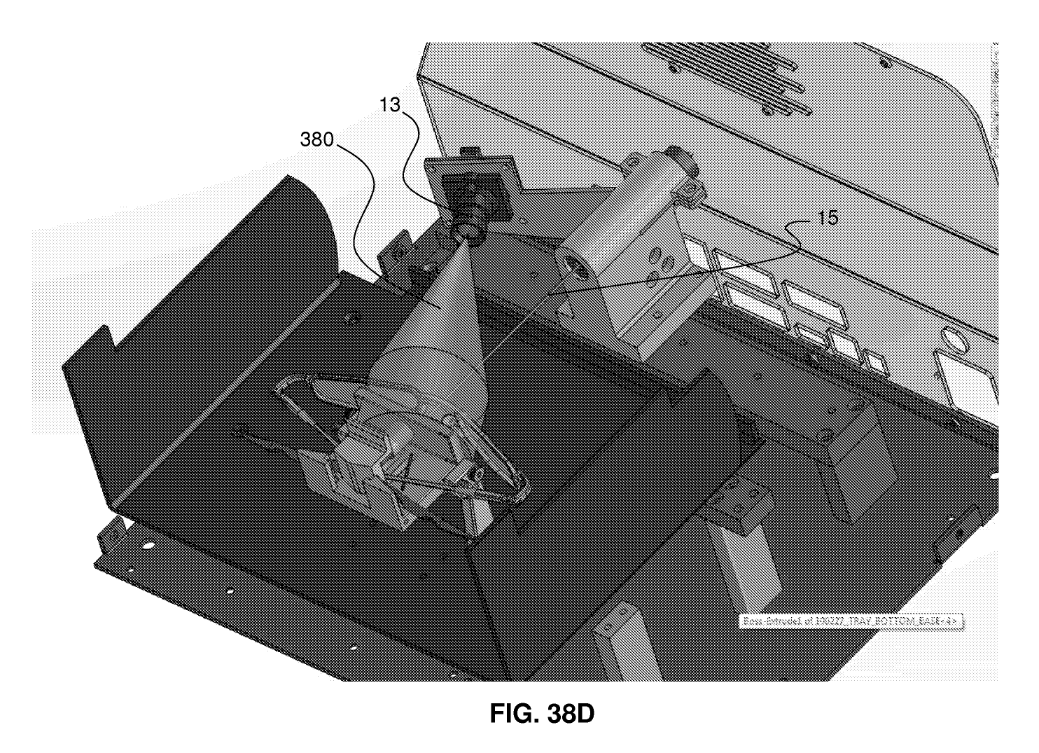

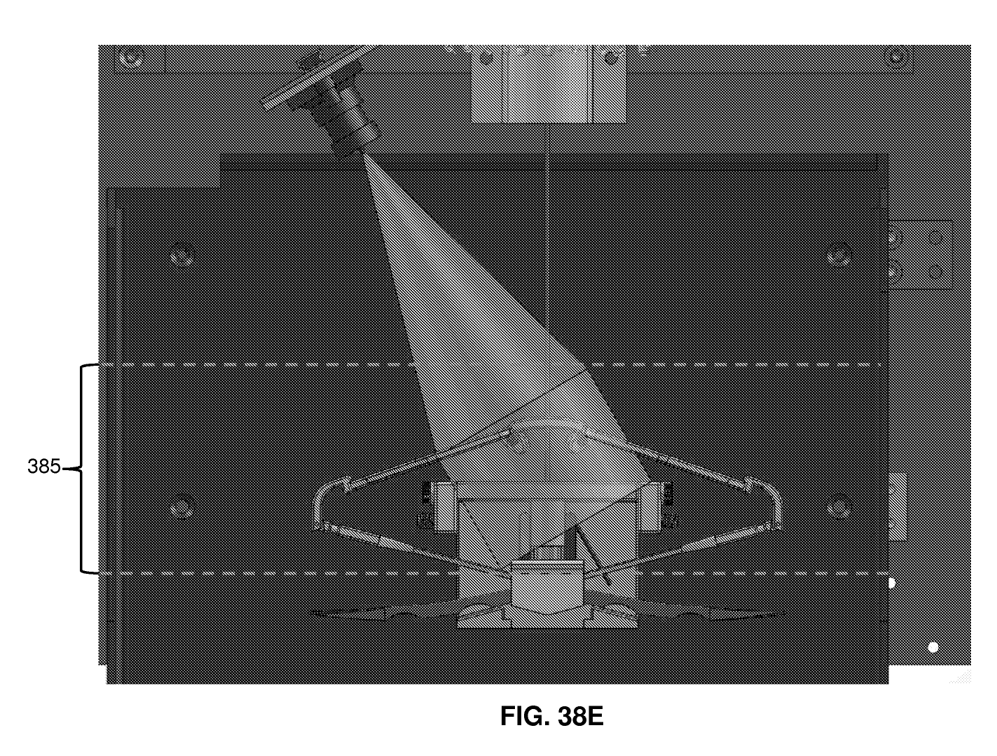

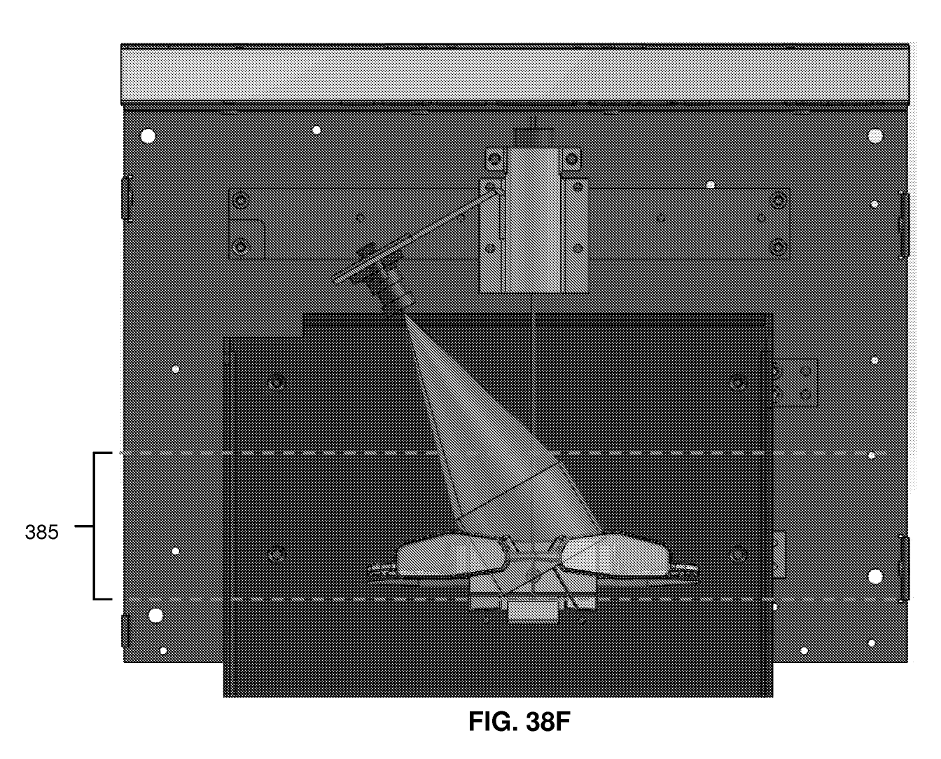

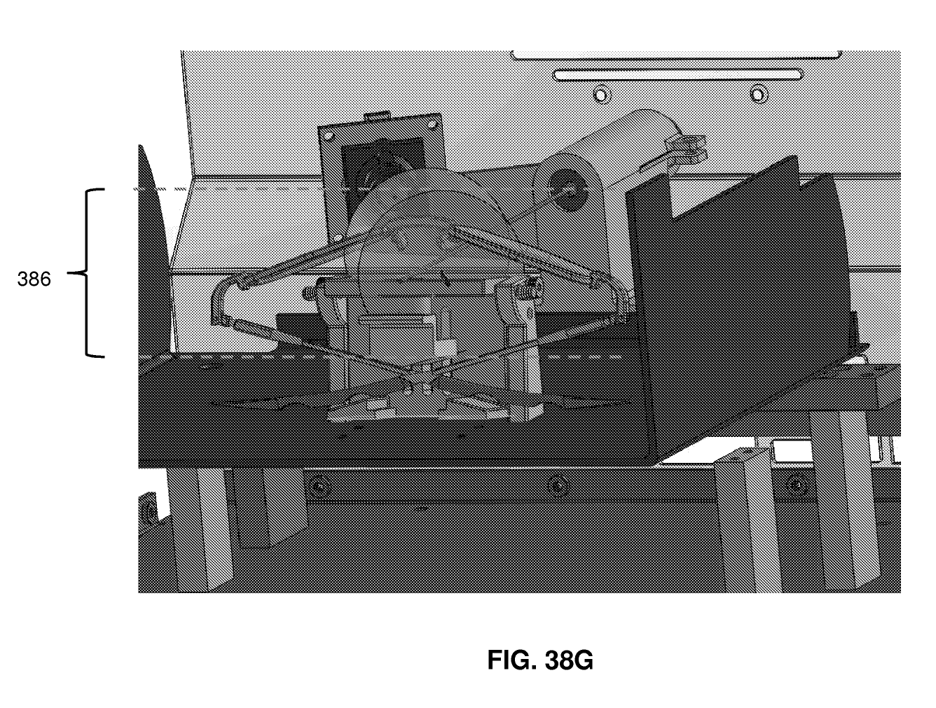

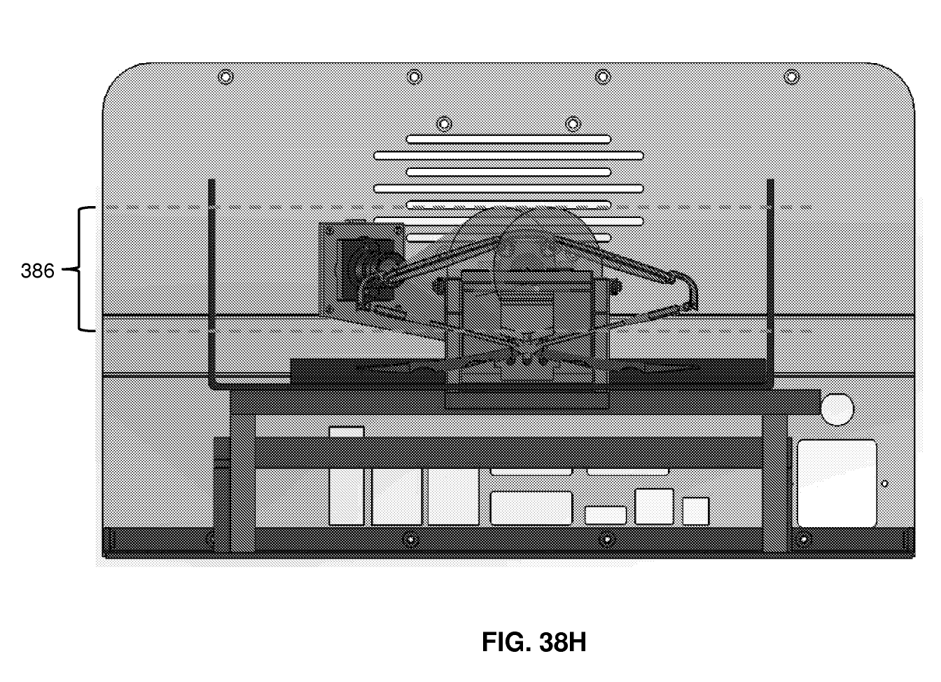

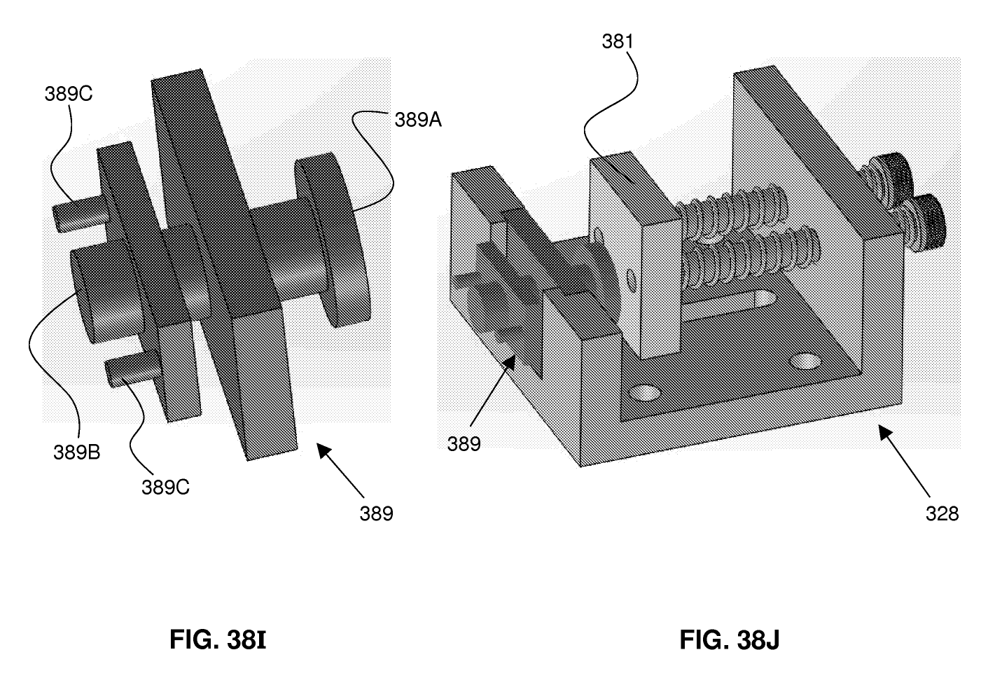

FIGS. 38A to 38J are views of the frame/lens holder 328. The frame/lens holder 328 shown in FIGS. 38A and 38B holds the frame in a stable position that is perpendicular to the camera/laser assembly 13, 14. To removably fix the lens/frame with respect to the camera/laser assembly 13, 14, the frame/lens holder 328 has a clamping mechanism comprising a first movable clamping surface 381 and a second opposing fixed clamping surface 382. A bias device 383 (e.g., a spring) biases the first clamping surface 381 in a direction towards the second clamping surface 382 such that, when the first is moved back to place an object therebetween and is let go, the object is fixedly held between the two surfaces 381, 382.

The frame/lens holder 328 holds the frames/lens in a stable position in relation to the camera/laser assembly 13, 14. The frame/lens holder 328 is configured so that the frames/lenses can be held perpendicular to the camera/laser 13, 14 or they can be tilted, for example, at +80 or −80 degrees in relation to the camera/laser 13, 14, as is shown in FIG. 38C. These positions are set by detents and are fixed. The frame/lens holder 13, 14 uses one or more spring-loaded and cushioned fingers, for example, to hold the ear pieces of the glasses frames. With an attachment, lenses can be held at either 0 degrees or up to 90 degrees in relation to the camera/laser 13, 14. The lenses are held by the attachment with an adhesive pad.

FIGS. 38B and 38C are side views of the lens and eyeglass frame holder. The lens or eyeglass frame is mounted on the holder 328 and can be rotated around the pivot point 384 to an 80 degree downward position so that the eyeglass frame bevel can be scanned by the laser line and viewed by the camera at the same time. This approach allows the measurement of the frame bevel or the lens bevel or lens groove for mounting lenses in metal zyl or fish-wire eyeglass frames. For drilled rimless frames, the laser scanning of the lens template where the laser is orthogonal to the lens plane also captures the drill holes information and/or notch information in case of prescription lenses with drilled holes already present in the lens so that the lens can be mounted in drilled frame temples and nose-bridge components. FIG. 38D illustrates the frame mounted in the holder 328 inside the 3D laser eyewear tracer and shows, with a transparent cone and cylinder, a field of view 380 of the camera 13 that is large enough to engulf the entire depth of the eyeglass frame while it is being scanned by the laser line 15. FIG. 38E shows from above the frame mounted in the holder 328 inside the 3D laser eyewear tracer, where the depth of field of view 385 of the camera corresponds to a region between the two dotted lines. The depth of field of view 385 is larger than the entire depth of the eyeglass frame, which is orthogonal to the laser line 15 and the camera 13, so that the lens curve and the frame wrap angle are measurable when the laser line 15 scans the eyeglass frame. FIGS. 38F and 38G shows the frame mounted in the holder 328 inside the 3D laser eyewear tracer with the frame rotated at the pivot point 384 by 80 degrees so that the frame bevel can be scanned by the laser line 15 within the camera's depth of field of view 385. The view in FIG. 38G shows the camera's diameter of field of view 386. FIG. 38H shows the 3D laser eyewear tracer with the laser line 15 and camera 13 located at the back and the frame holder towards the front. The camera's diameter of field of view 386 covers the entire bevel and the frame curve as the laser line 15 scans the frame and the camera 13 captures the scanned laser line 15 that interacts with the frame bevel.

FIGS. 38I and 38J illustrate an adapter 389 for a lens that is placed in the frame/lens holder 328 in one of two orientations. In the first orientation where side 389A faces the movable clamping surface 381, a non-illustrated lens is attached removably to side 389A with an adhesive, for example, an adhesive pad. In a second orientation where side 389B faces the movable clamping surface 381, non-illustrated lens templates having a standard hole pattern can be placed on the adapter with the hole pattern matching hole bosses 389C.

In this exemplary embodiment, the frame/lens holder 328 can be rotated about a pivot 384 to move the frame/lens at an angle to the camera/laser assembly 13, 14 but fixedly holds the frame/lens in a given position, e.g., substantially perpendicular to a left-right translation assembly (e.g., the linear drive mechanism 327), and the camera/laser assembly 13, 14 translates from left to right with respect to the frame/lens as well as pivoting/rotating about the frame/lens. In such a configuration, the frame/lens experiences no movement and, therefore, scanning errors that could arise from frame/lens movement (e.g., bouncing) are eliminated. The camera 13 is attached to the linear drive mechanism 327 and captures images of the laser line from the laser 14 as the laser 14 moves across the object being scanned. The computer 321 communicates to the camera 13, for example, through a USB connection. The laser 14 also is attached to the linear drive mechanism 327 and generates a laser line 15 (or a laser path such as shown in FIGS. 9, 11, 12, and 14) that is scanned laterally across the surface of the object being scanned as the linear drive mechanism 327 translates, for example, from left to right or right to left.

The inventive 3D laser eyewear tracer is capable of measuring frame wrap angle, lens base curve, and different types of eyeglass frames such as and not limited to:
a. metal zyl (where the lenses are mounted in metal frames and held by screws) with shiny or matte finish;
b. plastic zyl (where the lenses are mounted in plastic frames and held by screws) with shiny or matte finish; clear or translucent in color;
c. frames made in any material such as plastic, metal, wood, etc.;
d. fish-wire (where the lenses are mounted in frames and held in place using fish-wire);
e. drill-mounts (where the lenses have drilled holes where the temple and nose bridge are attached by screws and nuts);
f. adhesive mount (where the temple and nose bridge are attached to the lenses with adhesive); and
g. lens templates for drill and adhesive mounts including the hole diameter and hole angle; and
h. lens bevels such as U or V shaped, Top Hat, Flat, Groove, back angled bevel, etc.

Each embodiment of the 3D laser tracer described or shown herein employs software to convert the detected laser scan from the camera 13 into computer-readable data that, for example, follows the VCA format and, therefore, can be sent to any lens cutting device to form a frame-usable lens(es). One such software process utilizes a triangulation methodology that is illustrated in FIGS. 9 to 14. FIG. 9 outlines the methodology. In summary, the system includes a laser 15 that emits a laser line to illuminate an object 16. A detector, such as a camera 13, detects the illuminated object 16 and also detects the surroundings of the object 16 that are illuminated as well. The object 16, can be, for example, an eyewear frame or a lens or a pair of lenses. Measurements that can be taken include, for example, a measurement of the frame with lenses, a measurement of the frame and the lens bevel, a measurement of a single lens, and a measurement of a single lens groove. A computer 1 receives the detected data and, with software and/or hardware, processes the detected data with frame tracing software (that eliminates/disregards the surroundings) and converts the result into VCA DCS digital format.

The system shown in FIG. 9 can also be used to provide 3D laser scanning for other applications based on the triangulation methodology. In one embodiment, the hardware, which includes laser 15, detector 13, and software running on computer 1, uses a laser line. In one embodiment, the laser line is a green wavelength. A laser of any wavelength ranging from Ultraviolet to Visible to Infrared may be used for scanning purposes with the requirement that the camera detection system is sensitive to laser wavelength. In one embodiment, the detector 13 is a CMOS camera detector. In one embodiment, the software includes peak detection software, custom edge detection software, custom 3D data analysis software, and VCA DCS digital formatting software.

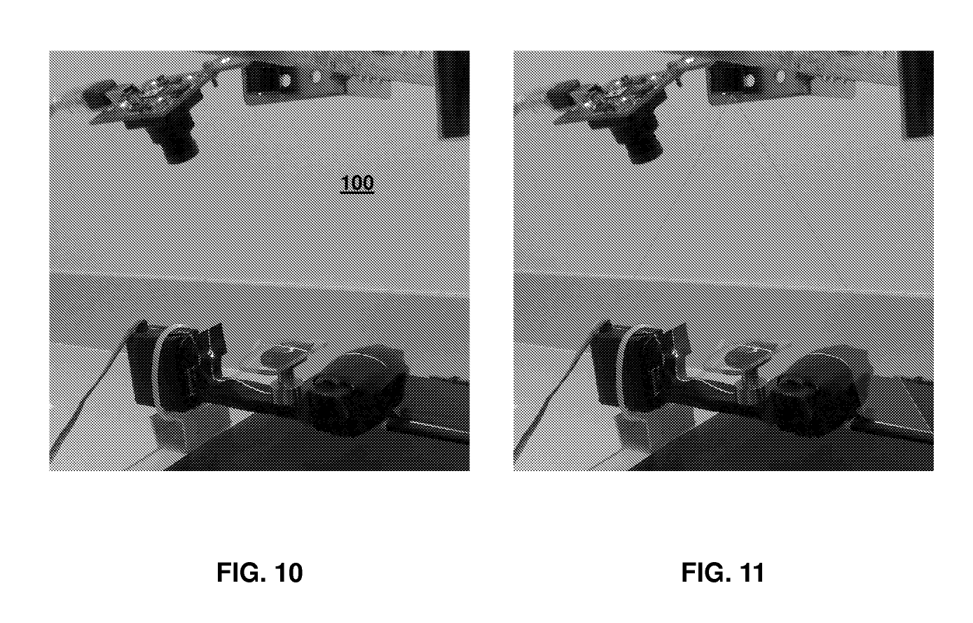

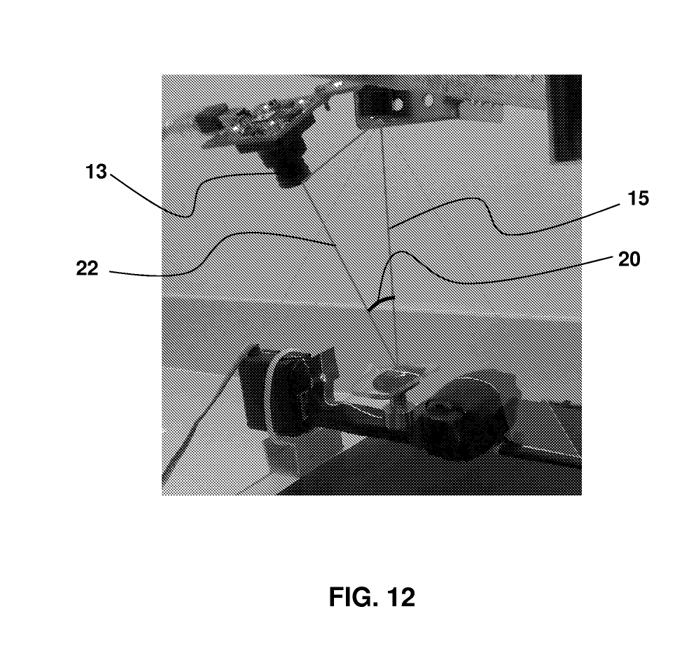

In FIG. 10, a laser line is generated across a single lens, here, in a top-to-bottom direction, and is scanned across the lens from left to right or right to left (with respect to the view of FIG. 10, in a somewhat front to back or back to front direction). The laser line is only shown in FIG. 10 at its termination point (e.g., on the tracer 100). The illustration of FIG. 11 shows the entirety of laser beam rotated/pivoted in the top-to-bottom direction so that a laser “fan” is visible. As can be seen, the laser beam illuminates not only the lens but also the surrounding parts of the lens holder (e.g., tracer 100) above and below the held lens. The embodiments of the tracers described herein are able to remove (via software) reflections from behind the lens (i.e., on the side of the lens opposite the laser) and from above and below the lens as the laser beam is reflected off of the tracer's components. The angle 20 formed between the laser beam 15 and the perpendicular axis 22 of the camera 13 (shown in FIG. 12) allows the software to apply the triangulation process and define points in a 3-dimensional space that, together, form a 3D representation of the lens (or the frame) being scanned.

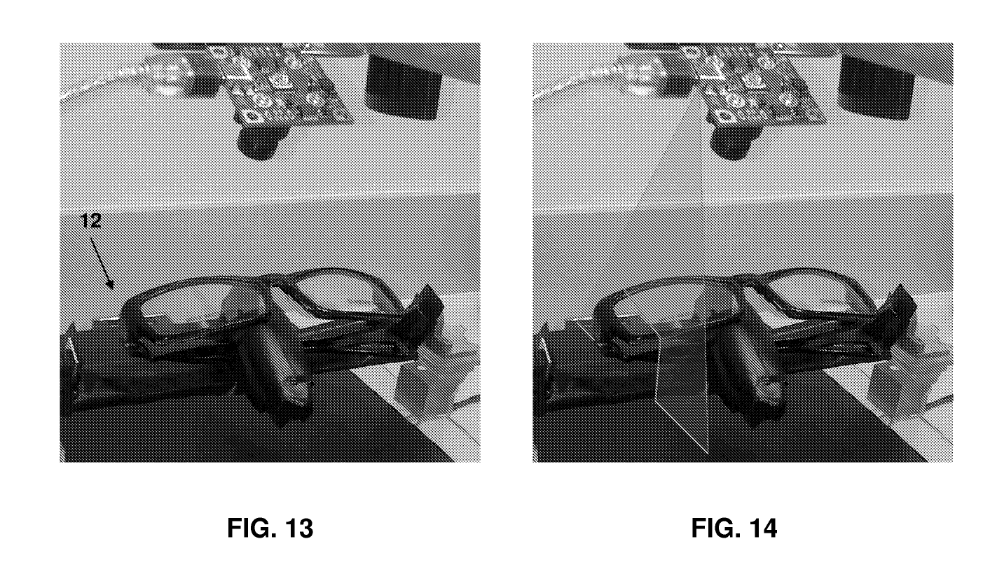

FIGS. 13 and 14 illustrate a similar scanning process for an eyewear frame having blank lenses. In this configuration, a frame holder 12 can tilt the frame with respect to the laser beam 15, as with the frame/lens holder 12 described above. This added tilt feature gives the 3D laser eyewear tracer the ability to most accurately measure the interior bevel of the eyewear frame.

In either configuration, it is beneficial to minimize movement of either the laser or the eyewear being scanned as one is being moved relative to the other. When the eyewear is the moving part, vibration can be encountered during the scanning process. When the laser is moving, in contrast, the holding stage holds the eyewear still as the laser assembly travels along, for example, a rail. Preloaded ball bearings between the rail and the stage eliminates rocking of the stage even though it is being moved in the Y-direction for scanning the eyewear.

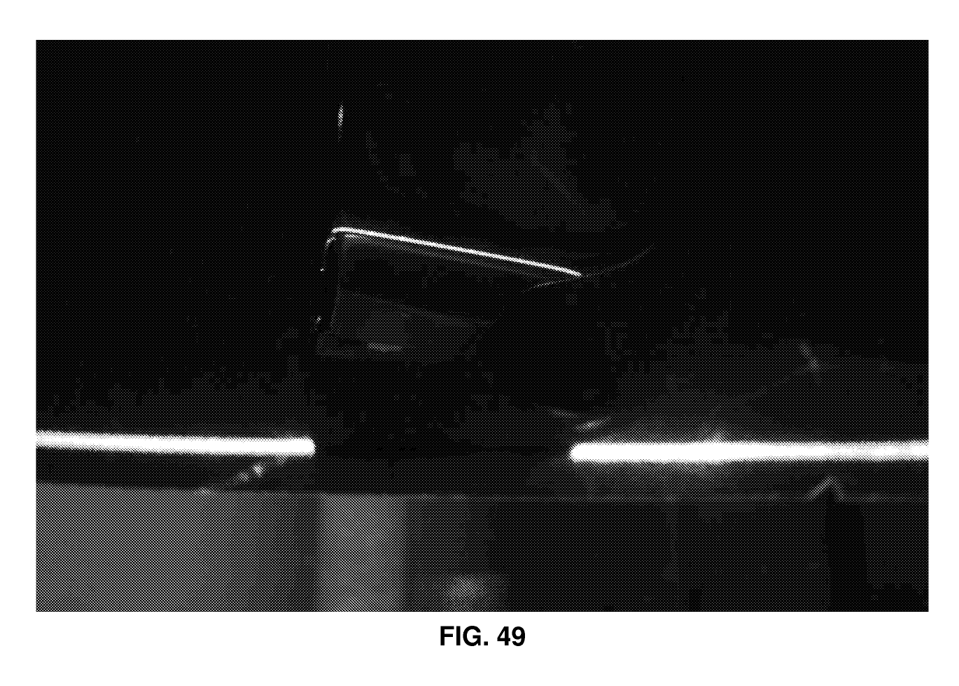

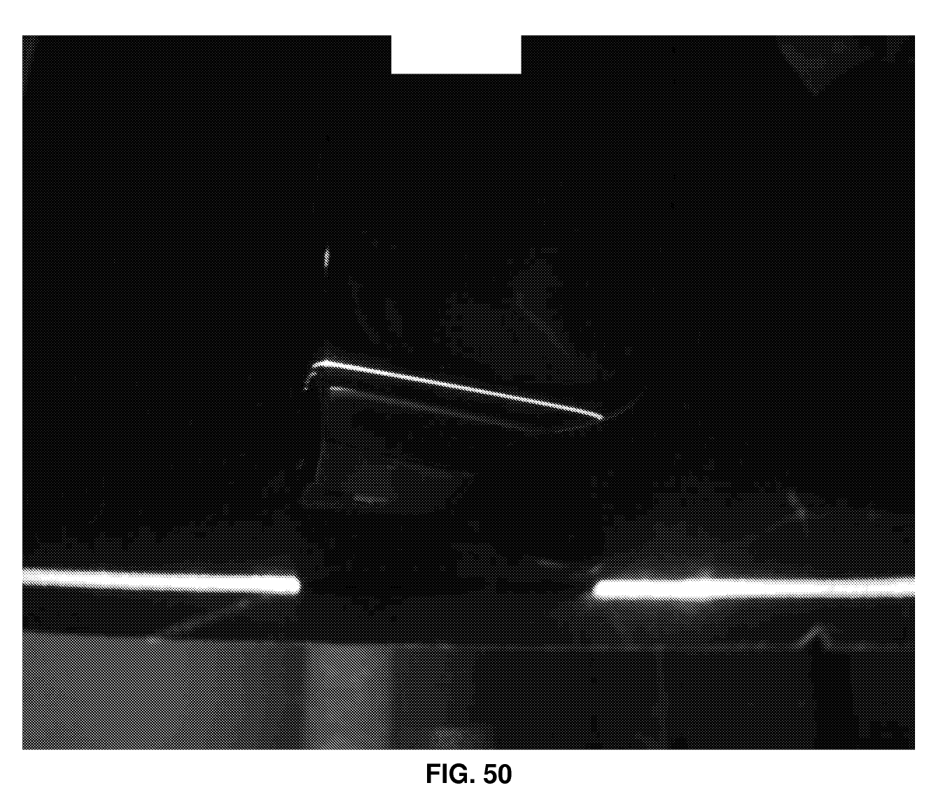

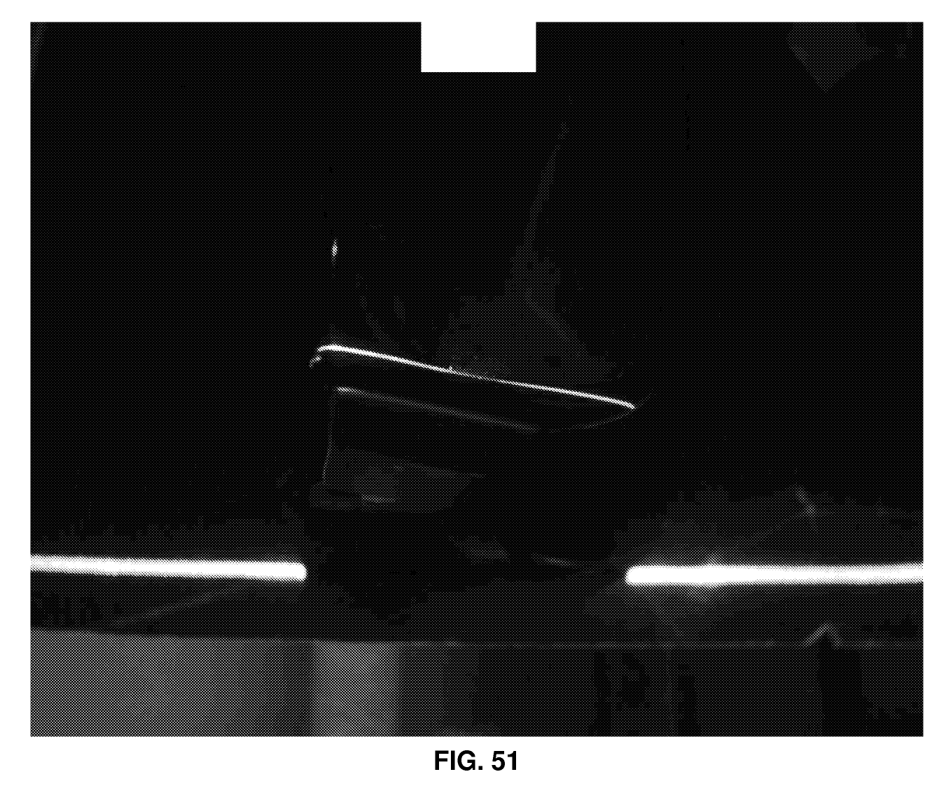

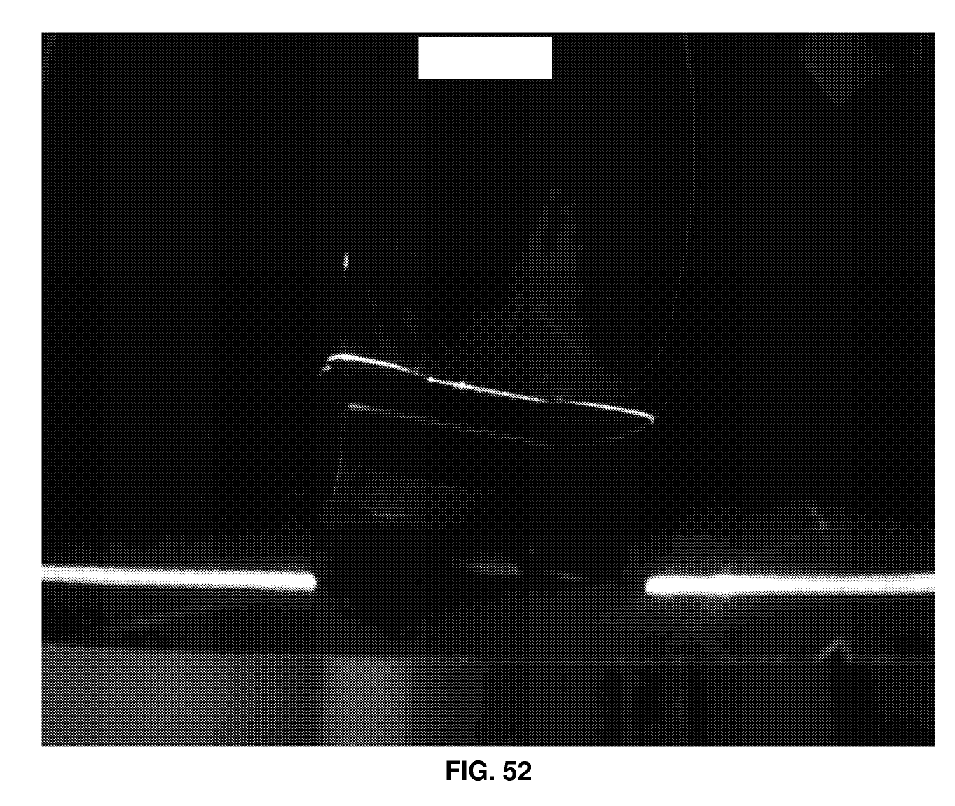

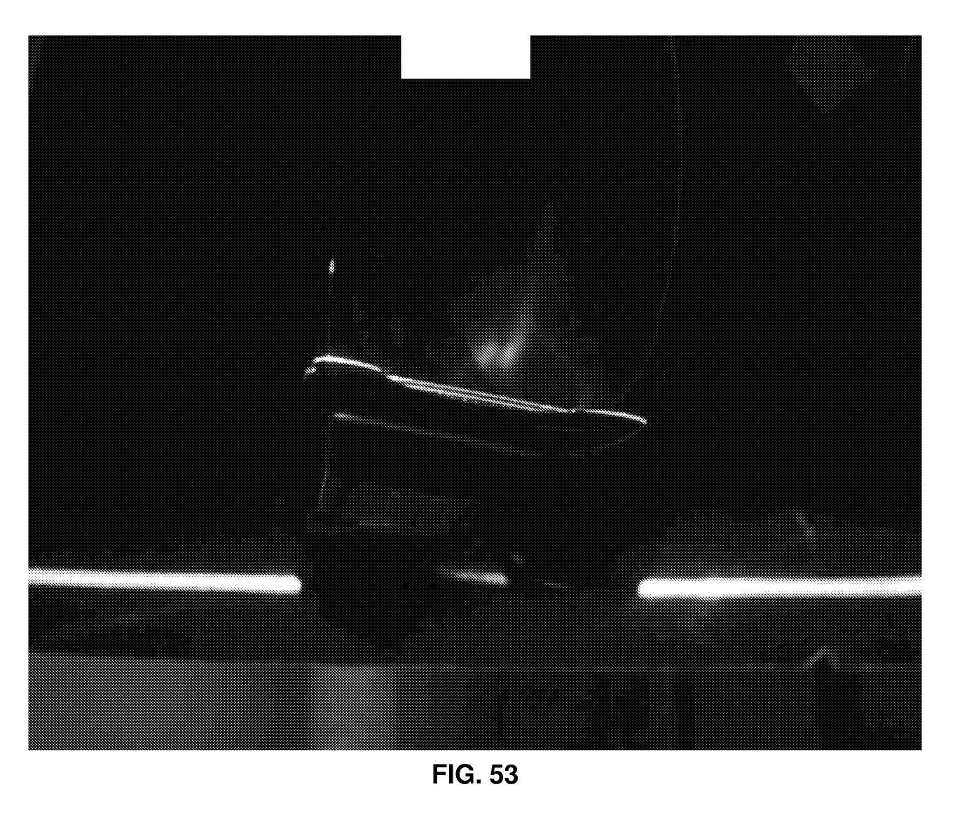

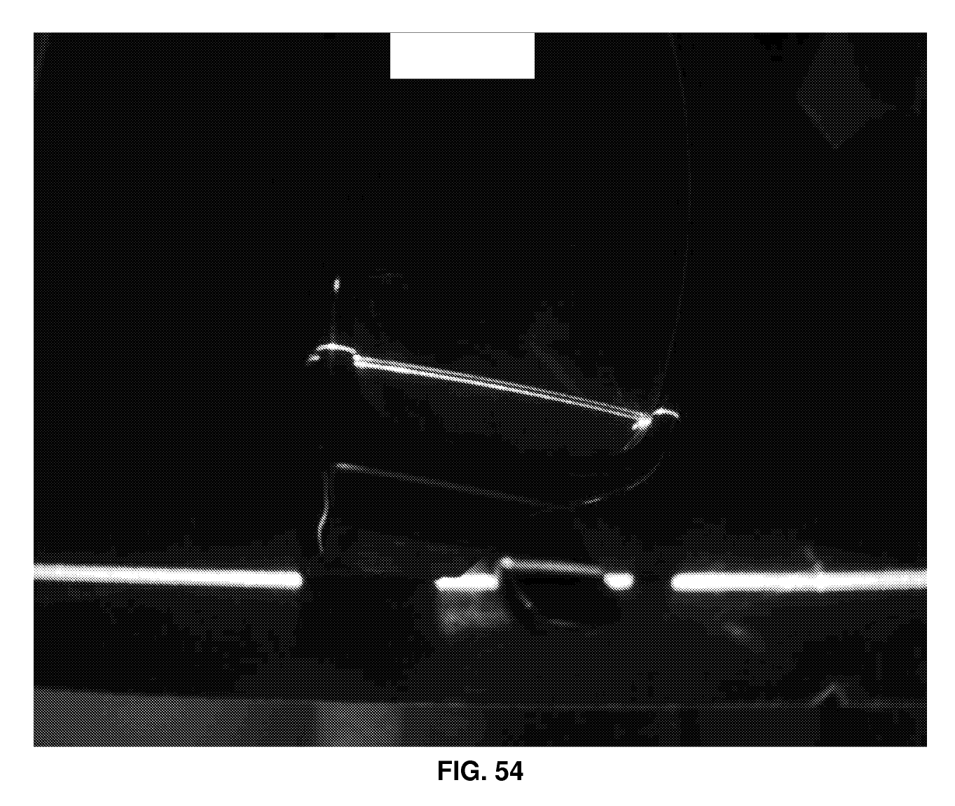

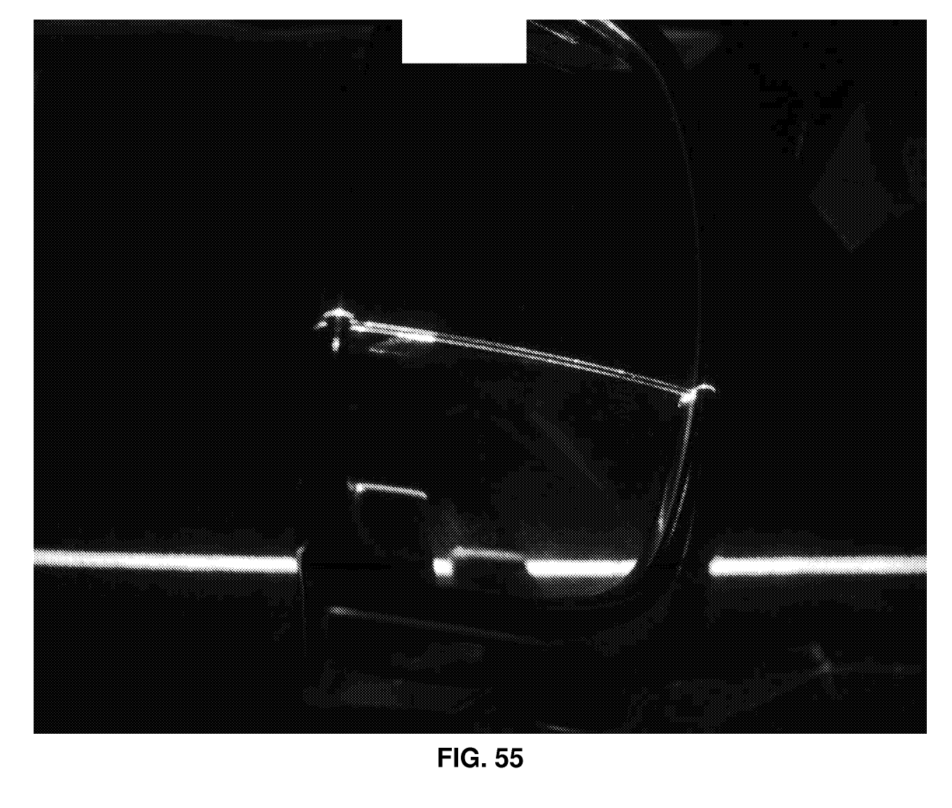

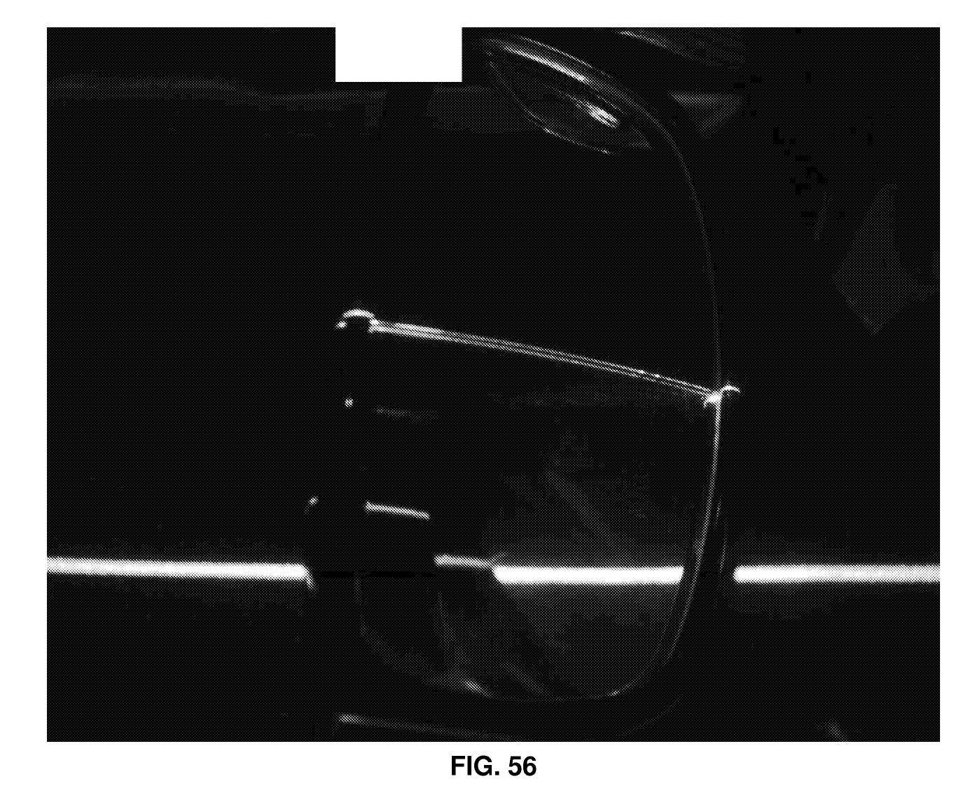

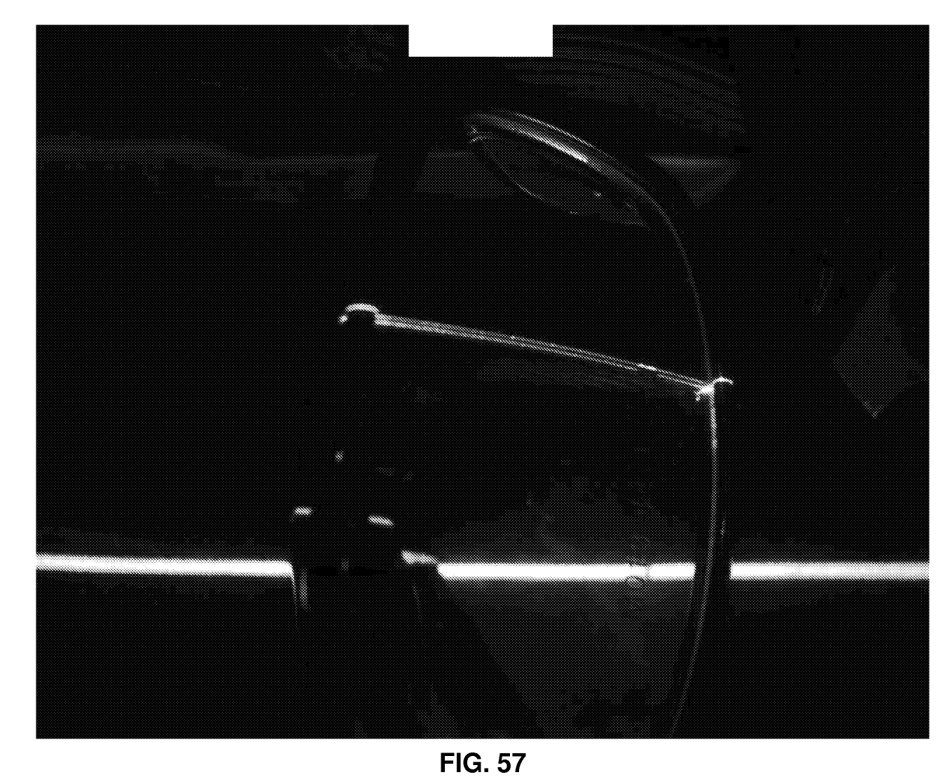

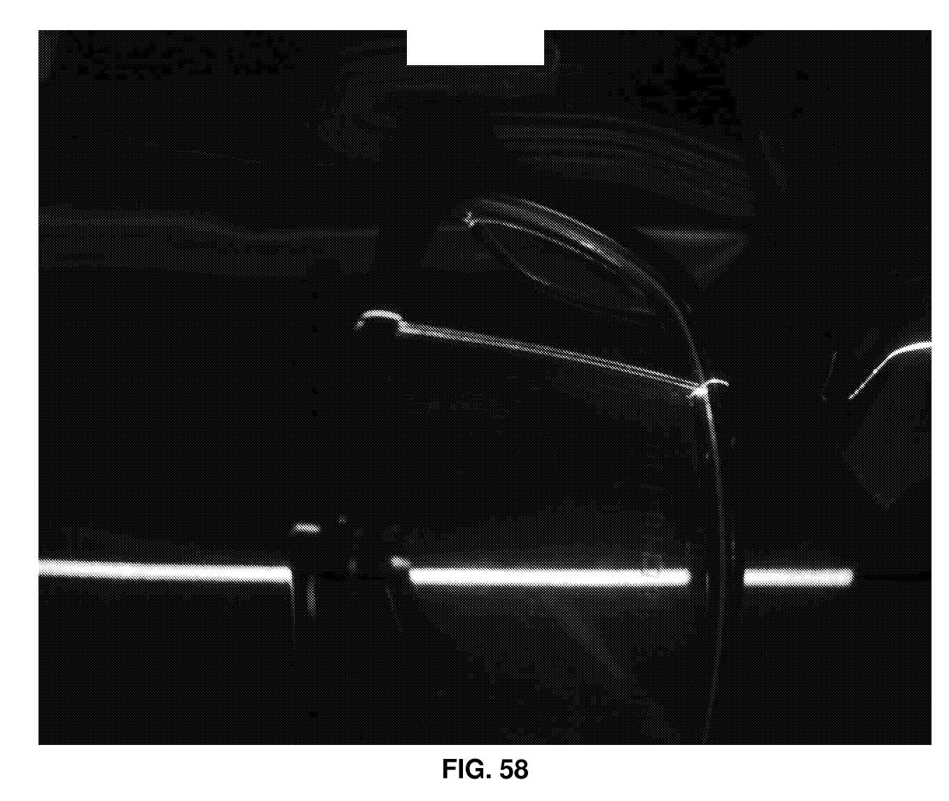

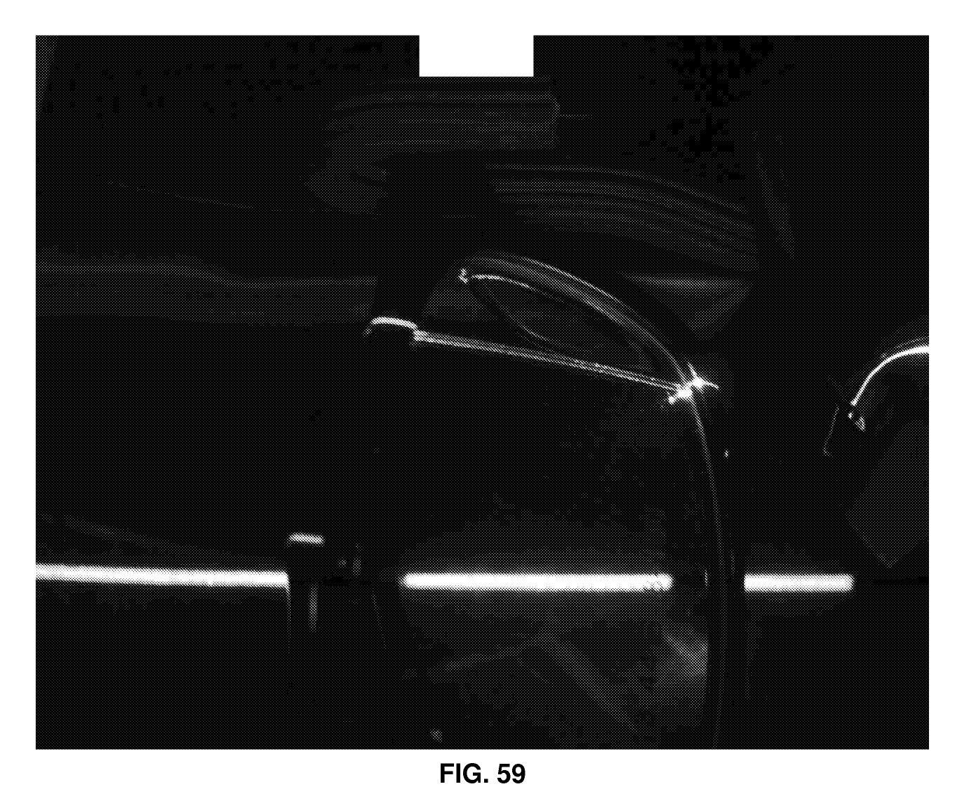

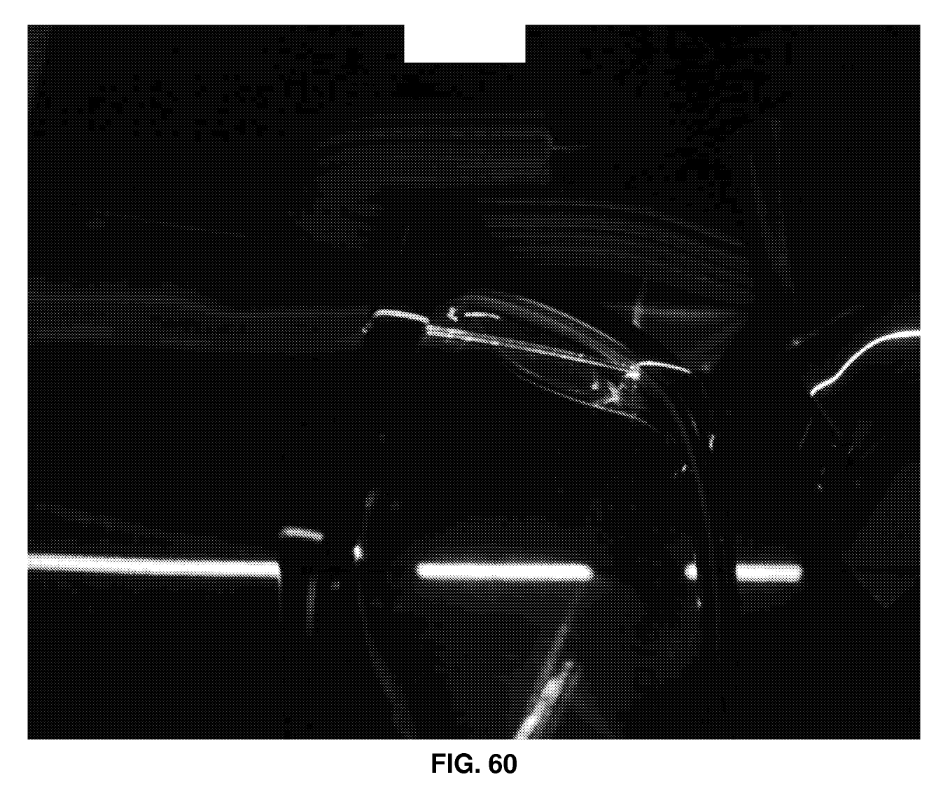

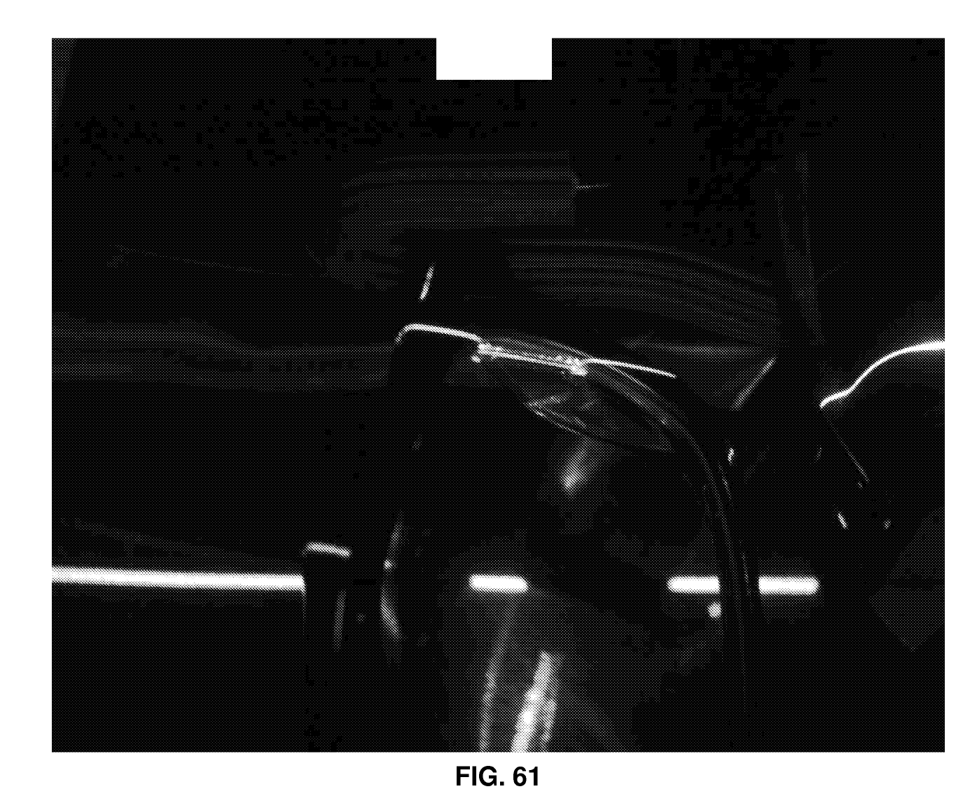

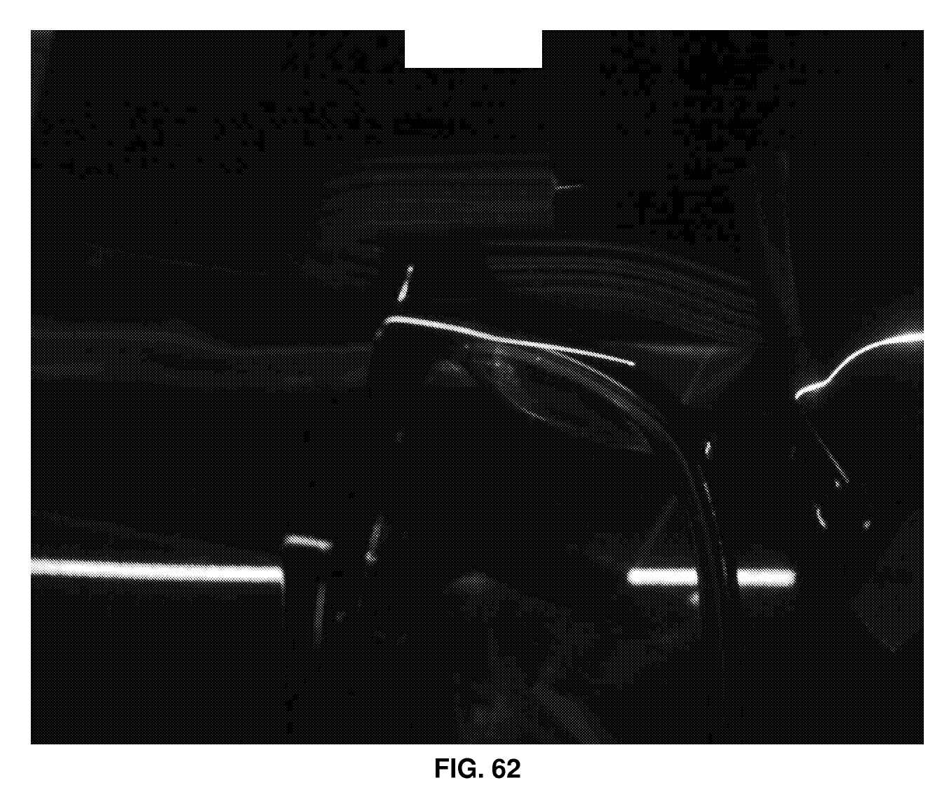

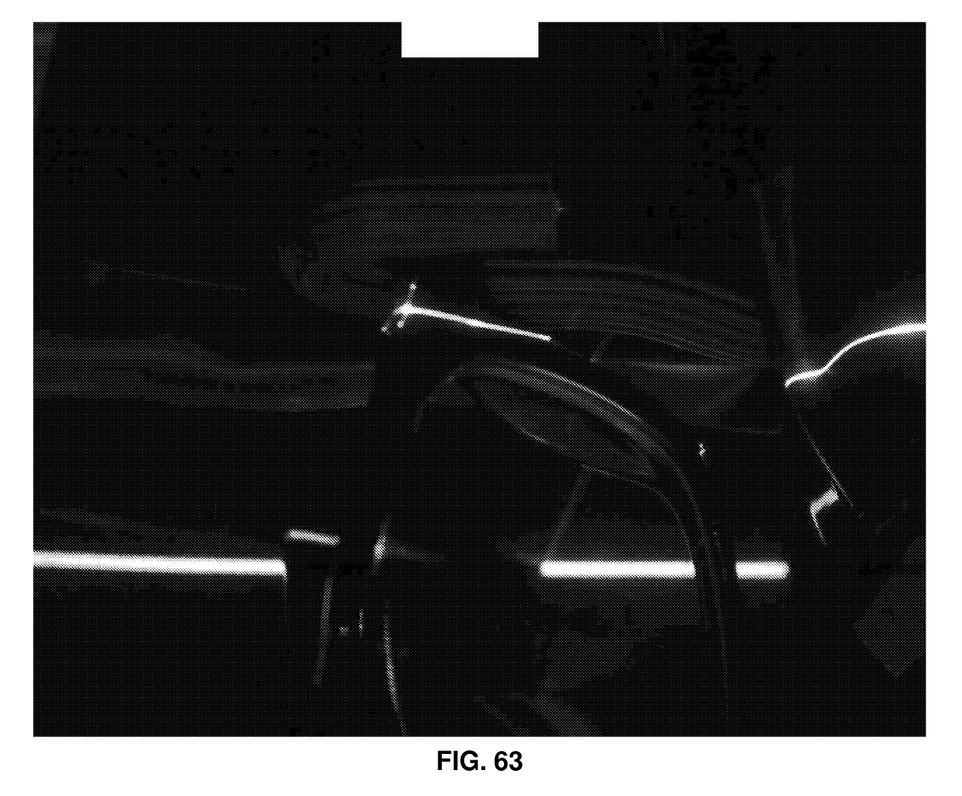

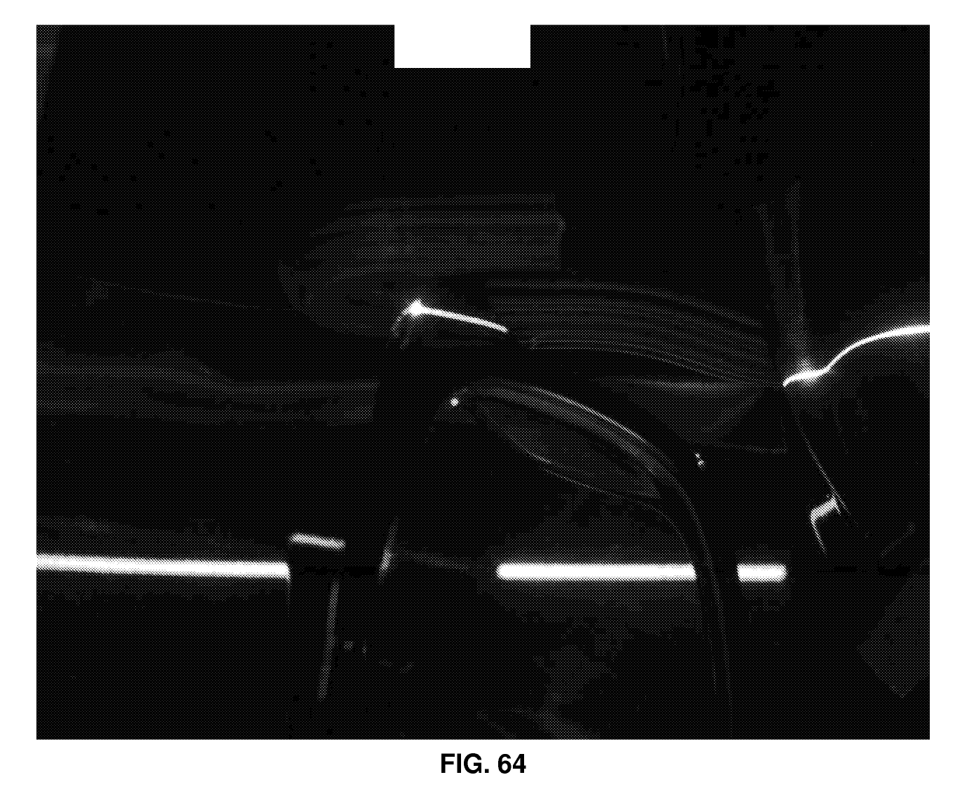

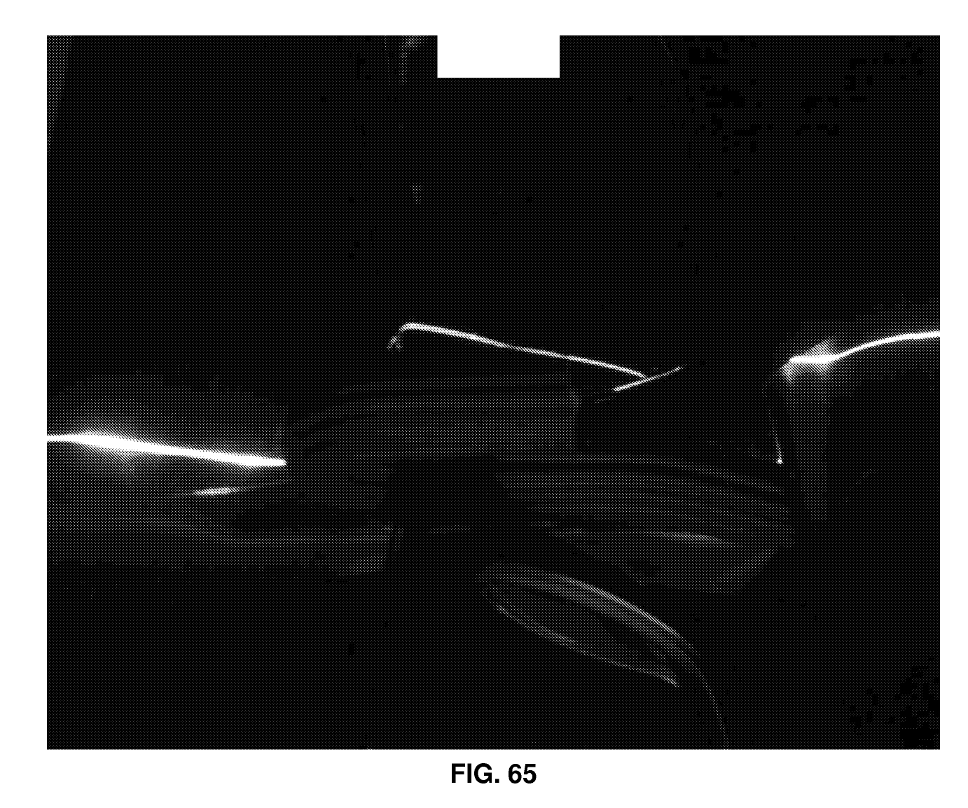

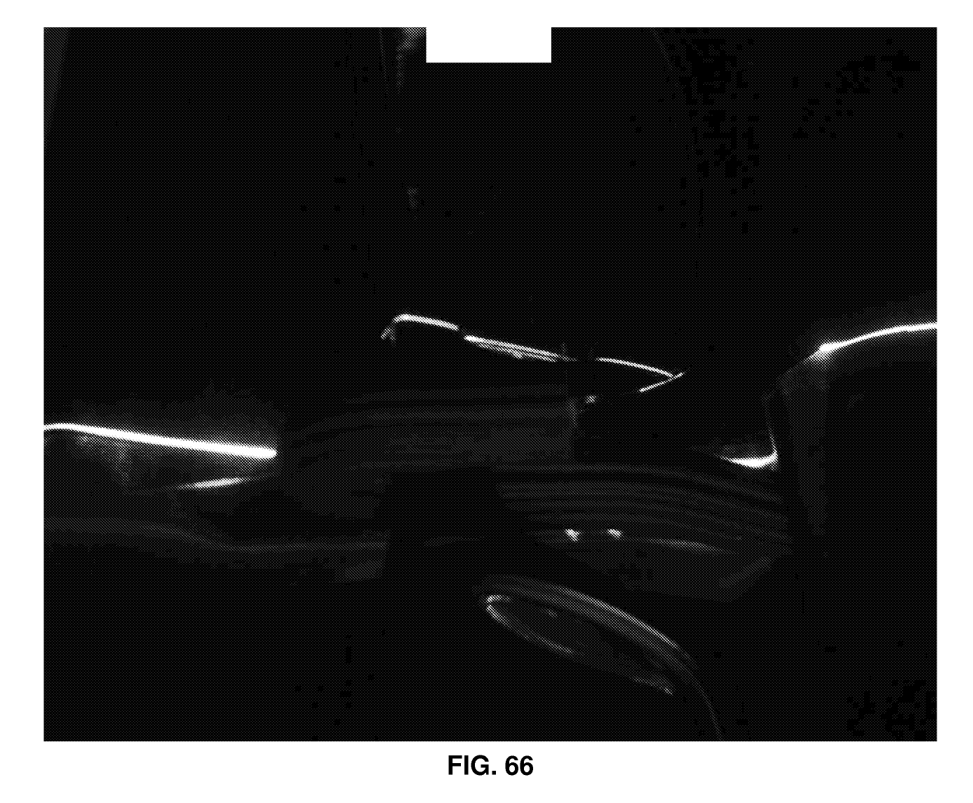

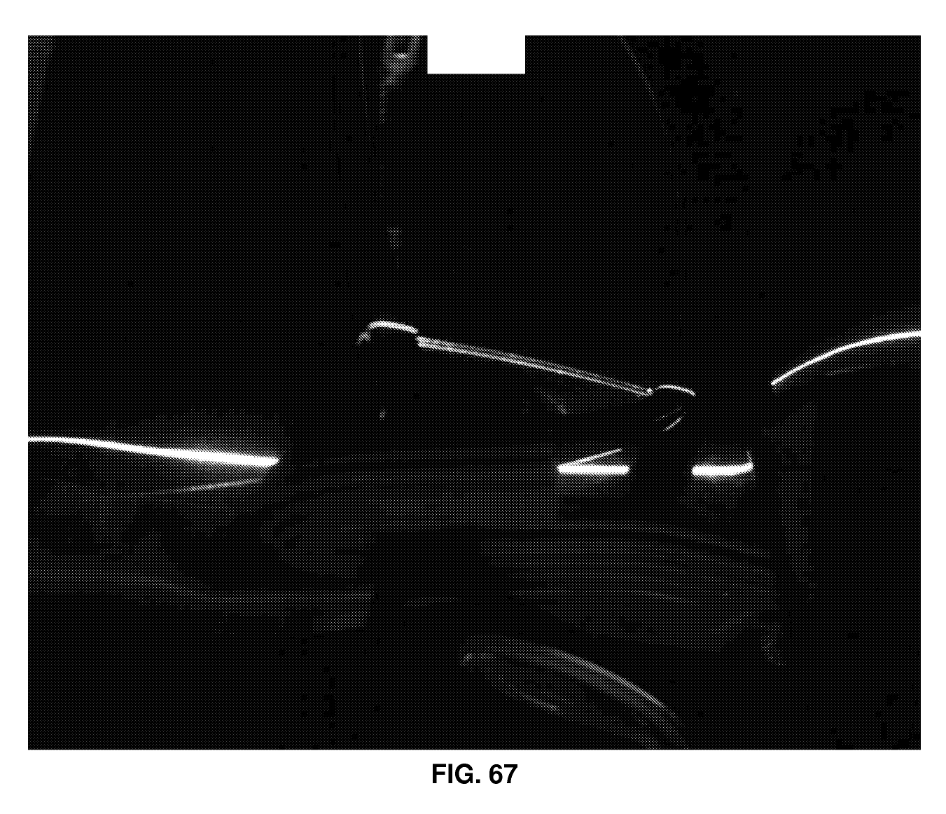

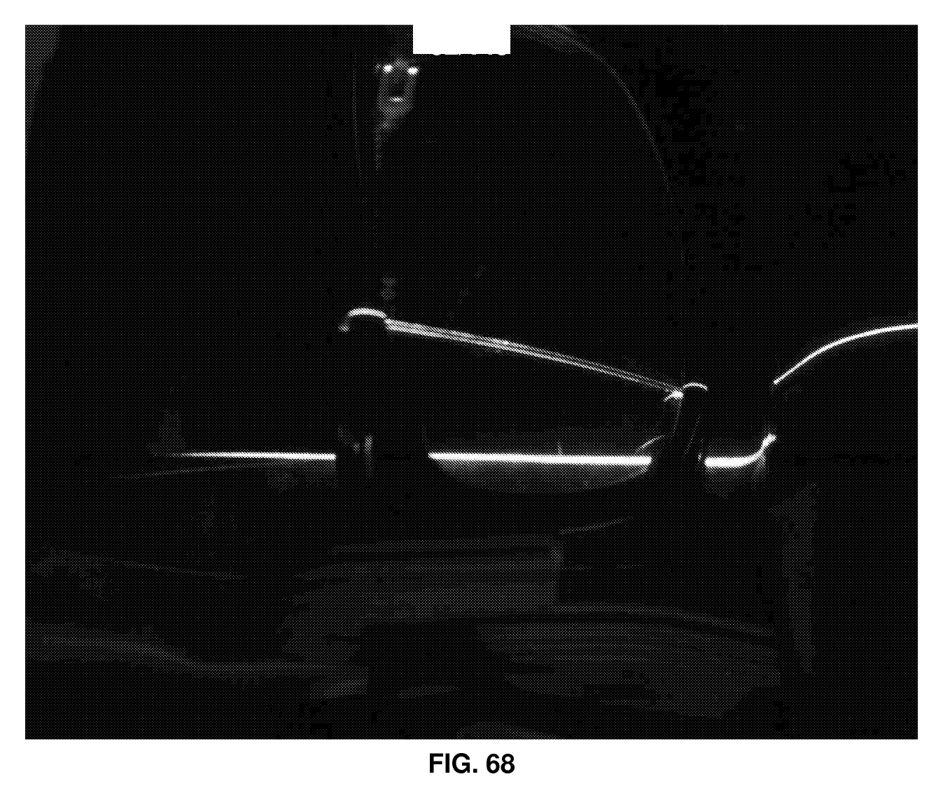

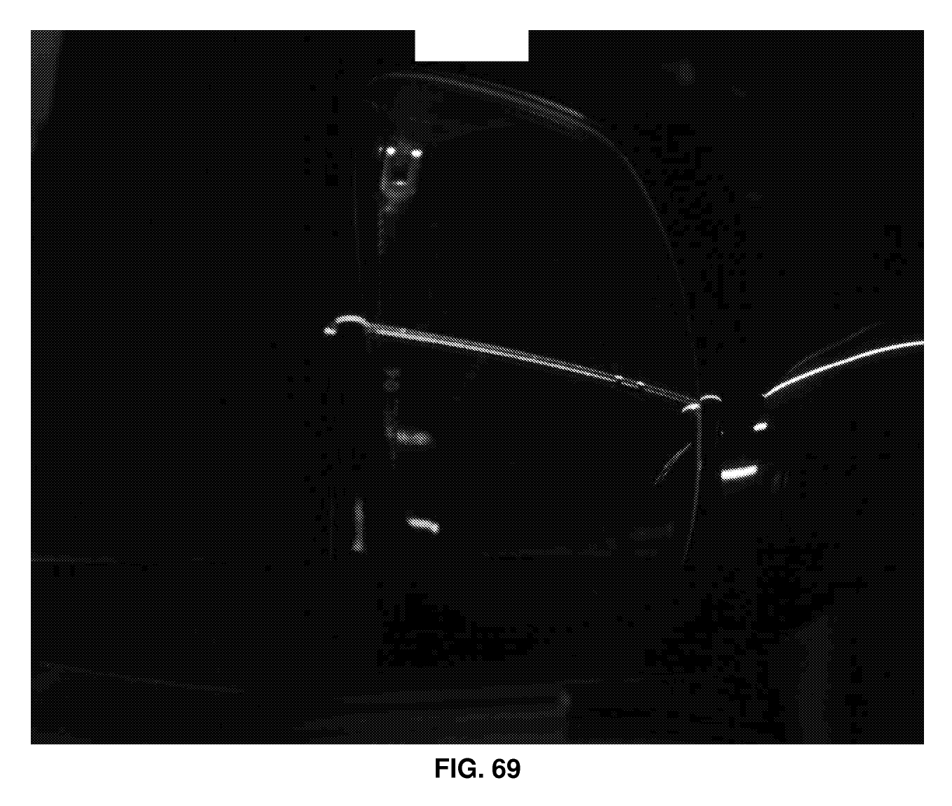

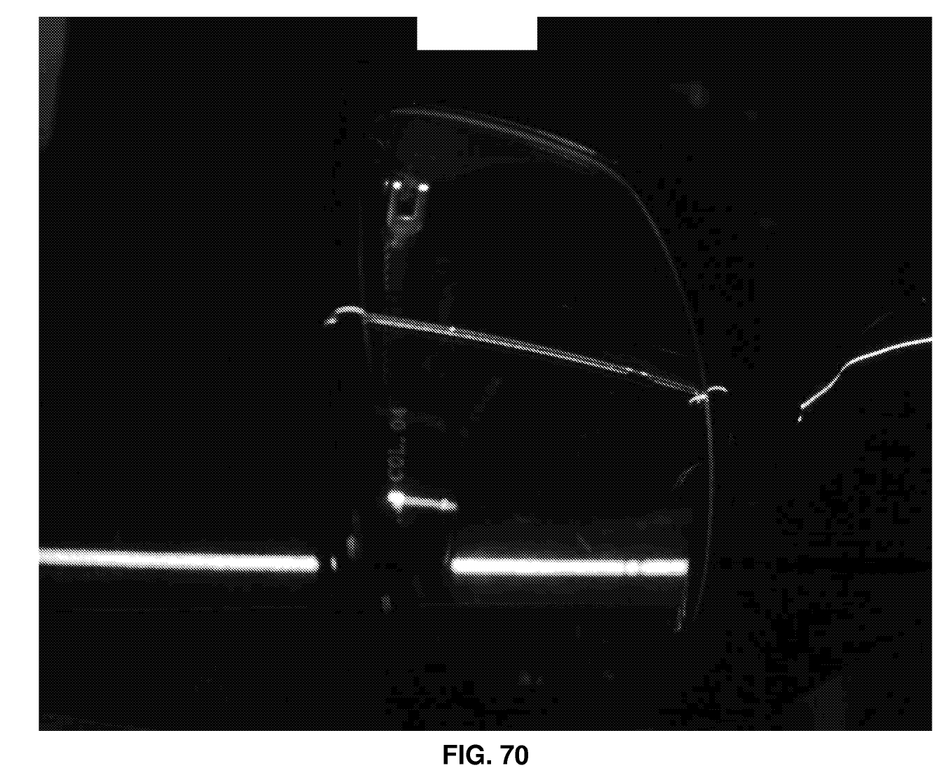

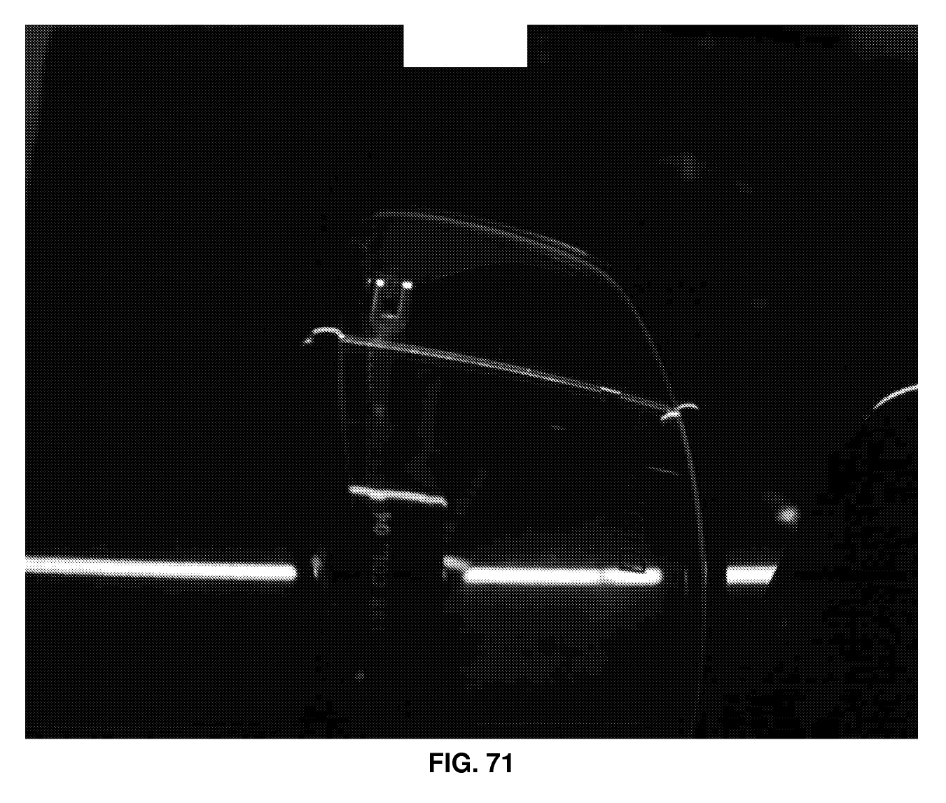

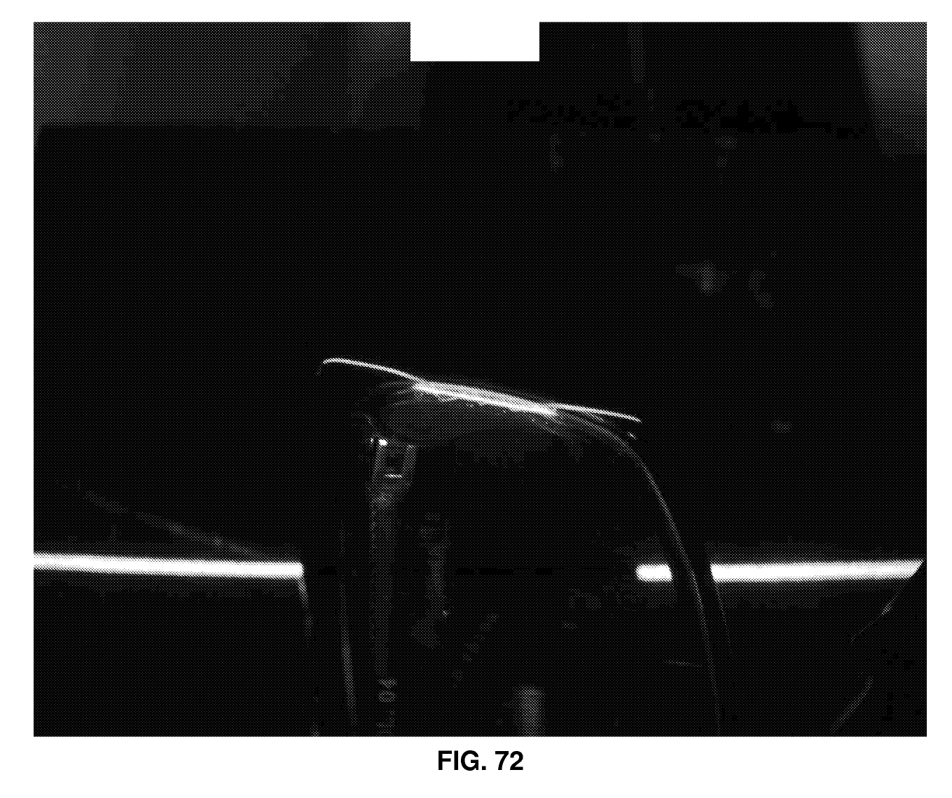

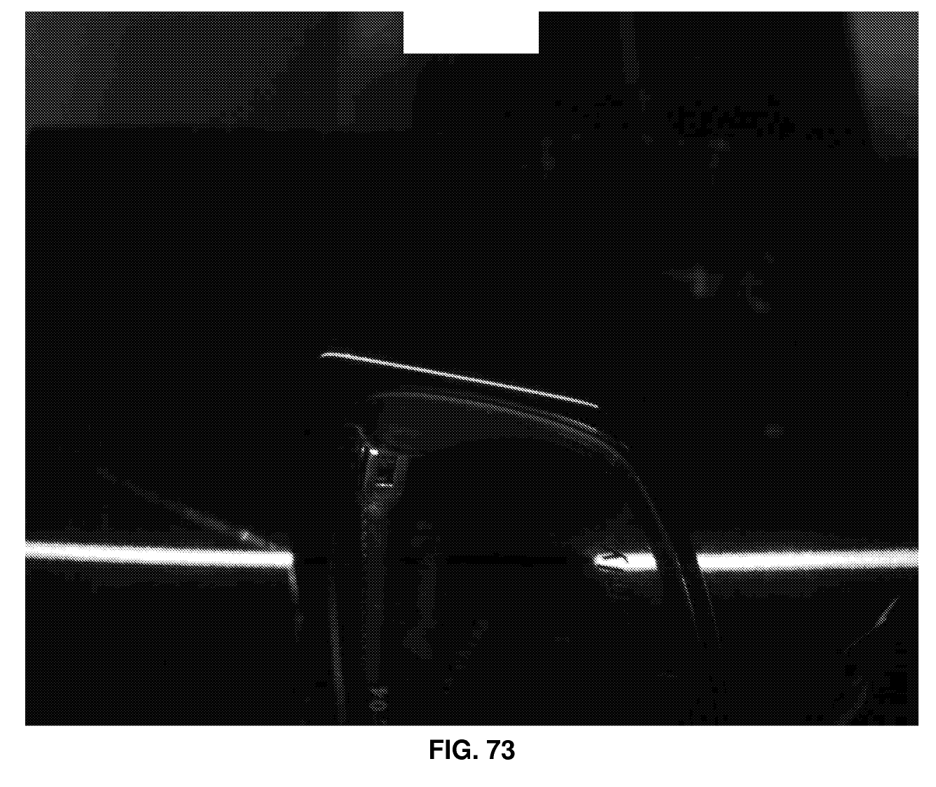

The 3D laser eyewear tracer is programmed with an ability to detect high and low laser reflection but to ignore the highest reflection where that reflection is from the device that is holding/supporting the lens to be scanned. This program is illustrated and describe with respect to FIGS. 49 to 73, for example.

Another exemplary embodiment presents the laser of the tracer at its lowest threshold to eliminate light piping through the lens being scanned. A further variable is the angle of the camera with respect to the laser line. This angle is selected to minimize reflection of the laser directly into the camera. Likewise, an angle of the camera with respect to the laser line is selected to minimize scattering of the laser directly into the camera. But, that angle is also maximized to increase the visibility of the surfaces being measured by the camera. In other words, the angle is optimized to increase signal-to-noise ratio.

In laser scanning technology, the laser line and camera are utilized to have the camera capture each individual scan as the laser scans over the object, in this particular case, a transparent optical element. The laser and camera are focused on the optical element and the angle between the laser and camera is set (typically at approximately 30 degrees) so that the two devices create a triangle to scan and provide 3D information of the optical element. There exists a problem with laser scanning transparent optical elements, however—transparent elements reflect laser light when such light impinges orthogonally on the element's surface. To overcome this problem, one prior art solution coated the optical element with opaque material prior to the laser scanning process. This potentially reduced scattering and reflection from the optical element but it introduced a new layer on top of the transparent optical element, which layer caused the laser and camera to provide incorrect dimensions of the frame/lens.

One approach that is employed to overcome this problem is to place a colored background (i.e., black paper) behind the optical element. Then, the element is scanned using the laser line and camera and the resulting data collected by the camera has the triangulation methodology applied to determine the 3D information, radius, or other desired parameters of the optical element. Having this colored background causes the laser light not to reflect strongly from the background and, in turn, significantly reduces the scattering and reflection of the impinged laser light on the optical element.

Yet another exemplary process that overcomes the scattering and reflection problem is to implement an algorithm during the image capture process that properly differentiates the signal from the noise and, thus, help reduces the laser scattering or reflection from the optical element. This process sets the desired Region of Interest (ROI) in the x, y and z dimensions so that the frame/lens template held in the frame holder is scanned in the desired ROI. The rest of the background is eliminated so that there is no additional noise observed in the scanned region of the frame or lens template.

Yet a further process that overcomes the scattering and reflection problem is to change the angle of the laser beam so that it impinges on the optical element from an angle that is different from orthogonal to the optical element while still meeting the angular requirement between the laser and the camera. During the scan, the frame can be tilted so that the reflected light is weaker in intensity and the camera is able to capture the desired frame information. Alternately, two cameras can be included in the laser 3D scanner, each of the two cameras being located on either side of the laser. The reflected laser light from the frame or lens template is detected by both cameras, whereby only one camera captures most of the desired data as the reflected light is outside its viewing angle. In this way, accurate data can be captured. The other camera detects the reflected laser light that is directly in its field of view and, thus, this data introduces poor signal to noise ratio.

Yet another process that overcomes the scattering and reflection problem is to optimize the laser intensity and the camera parameters so that the laser scanning does not produce the scattering and reflection problem. Each frame/lens template reflects light. The reflected laser light from a shiny metal or plastic frame is higher if the intensity of the laser is high and vice-versa. Alternately, a peak detection algorithm has the capability to isolate the highest Z-value signal, strongest Z-value signal, or lowest Z-value signal. This helps in the detection of front or back or most reflected surface of the frame/lens template accurately.

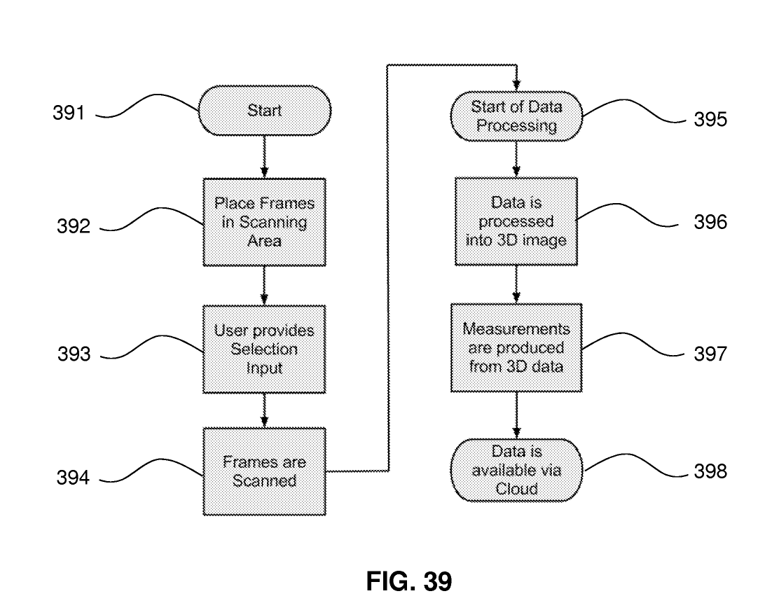

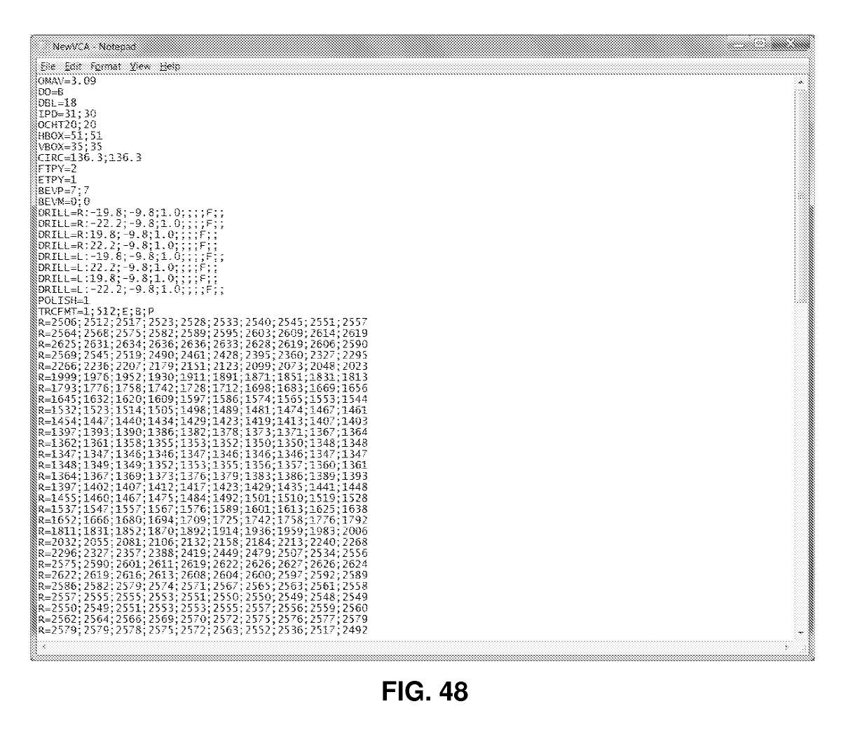

A process for scanning either a single eyewear lens or an eyewear frame is described with regard to FIG. 39, the frame being the example illustrated. Substitution of “lens” for “frames” in FIG. 39 carries out the lens-scanning process. After starting in step 391, the user places, in step 392, an eyewear frame in the scanning area, such as in a holder 12 within the scanning tray/drawer. The user selects the type of scan to be performed and any necessary input in step 393. The frame is scanned in step 394 and the tracer pre-processes the received data in step 395. The received data is, in step 396, processed into a 3D image. In step 397, the 3D image is able to produce measurements for cutting lenses for that scanned frame. These measurements are made available to any lens cutting lab directly or through the cloud in step 398. In an exemplary embodiment, the measurements are in Vision Council of America (VCA) Data Communication Standard (DCS version 3.09 or backward compatible with 3.06 or 3.02) format, such as the data shown in FIG. 48. The data contains lens circumference, distance between lens (DBL), inter-pupillary distance (IPD) for each eye, horizontal box, vertical box, bevel, drill information, and polish information and the trace format in 512 to 1024 data points that are read by lens edger to edge the lens. The lens edger is compatible with the VCA DCS and reads this data directly to edge and bevel the lens for each eye or simply the lens itself in case of drill mounted frame to hold the lens.

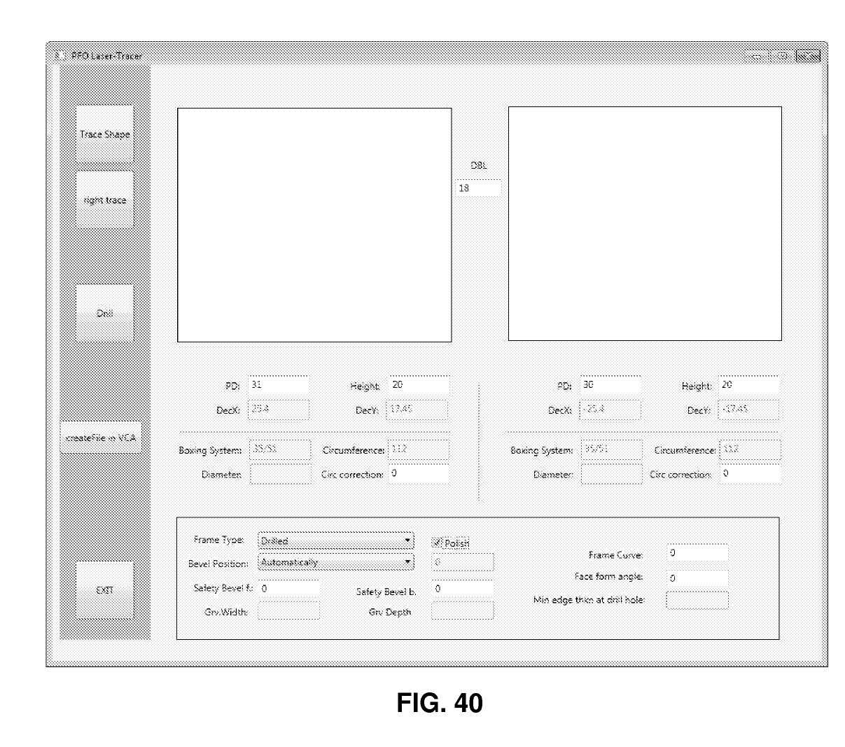

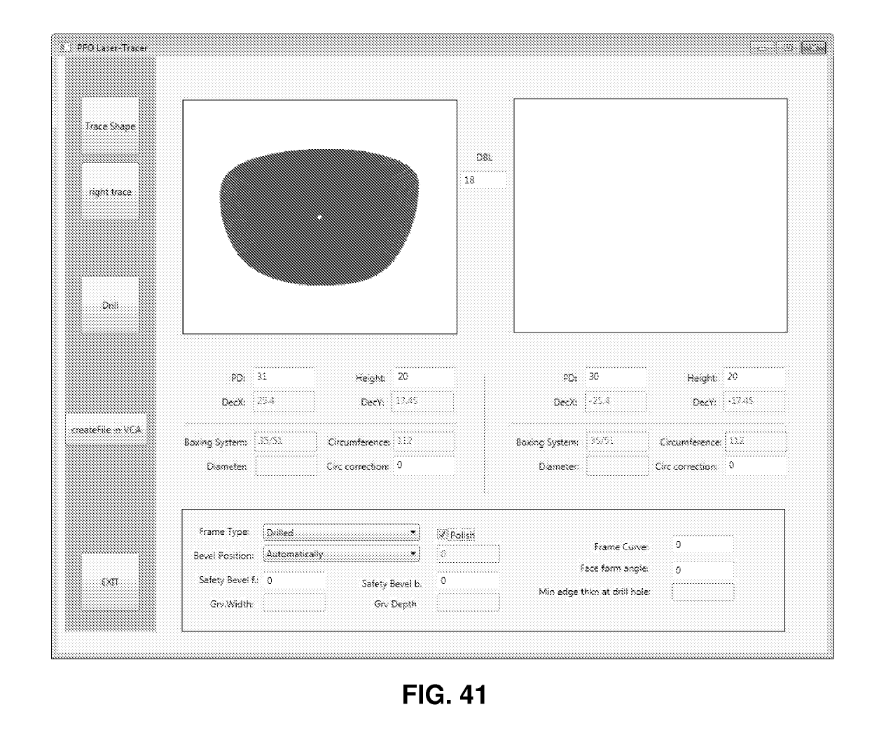

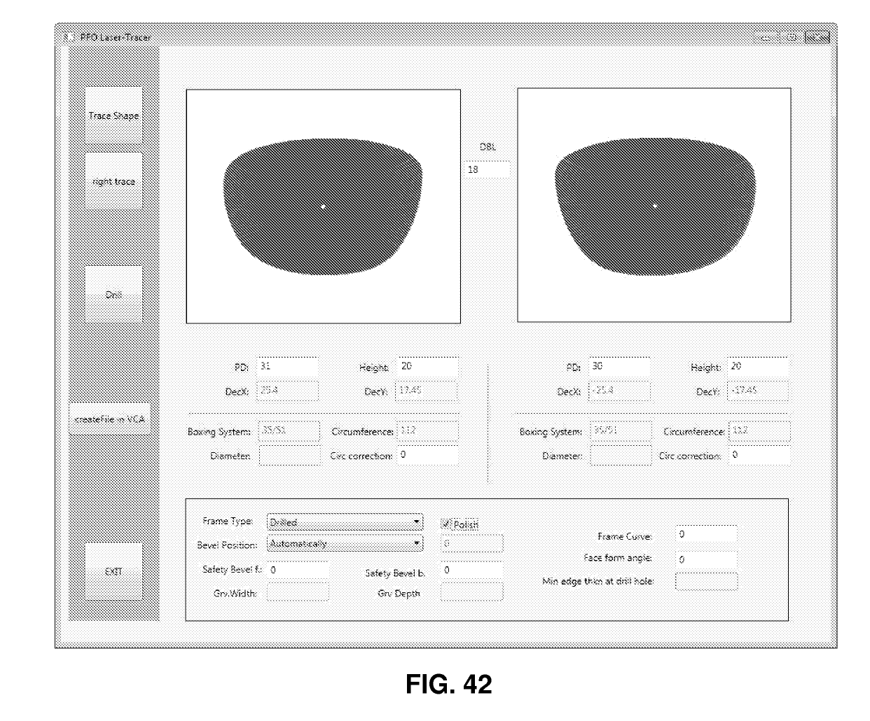

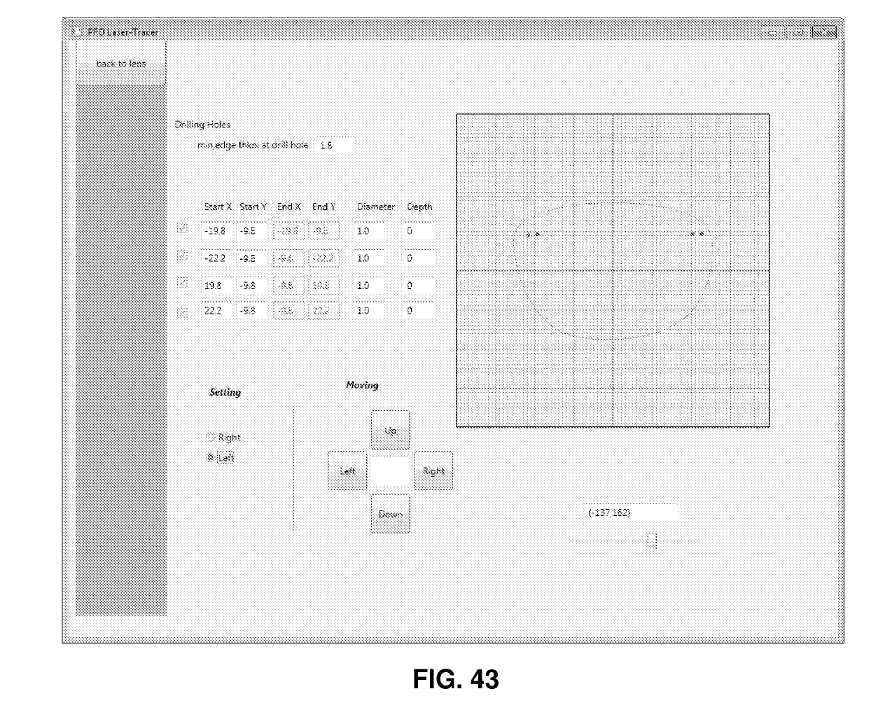

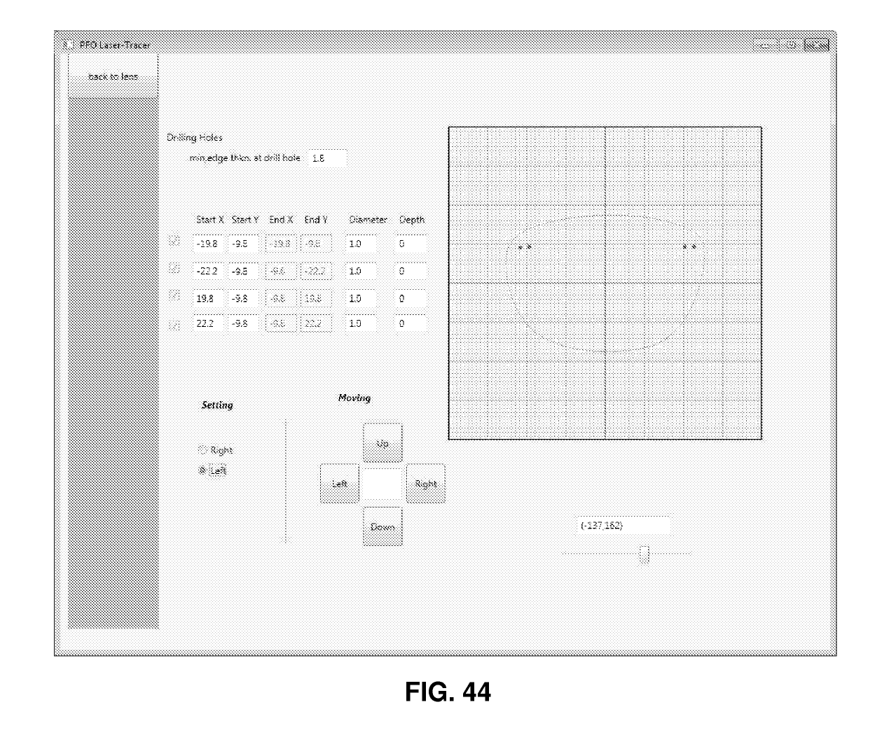

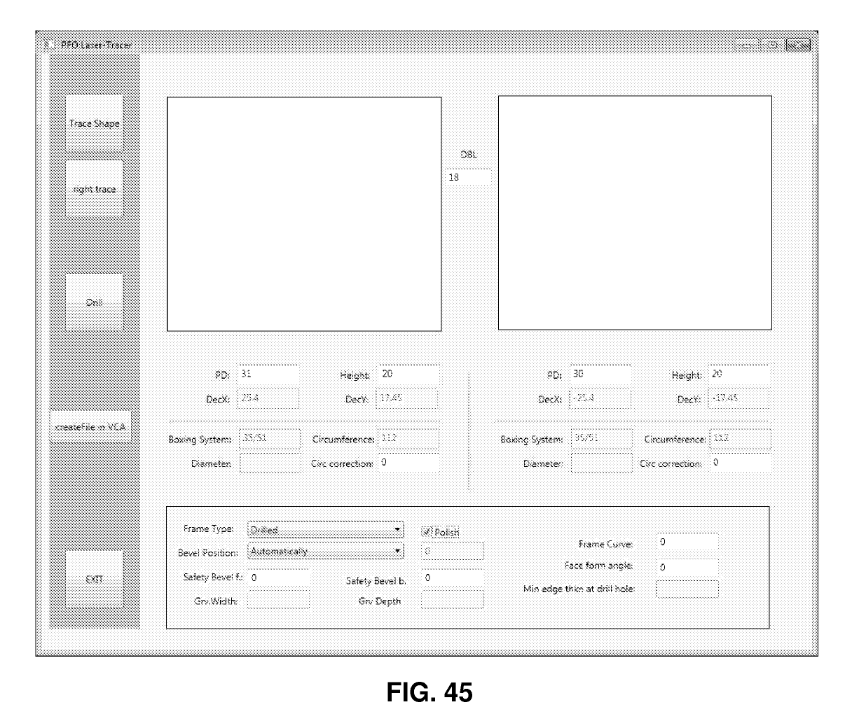

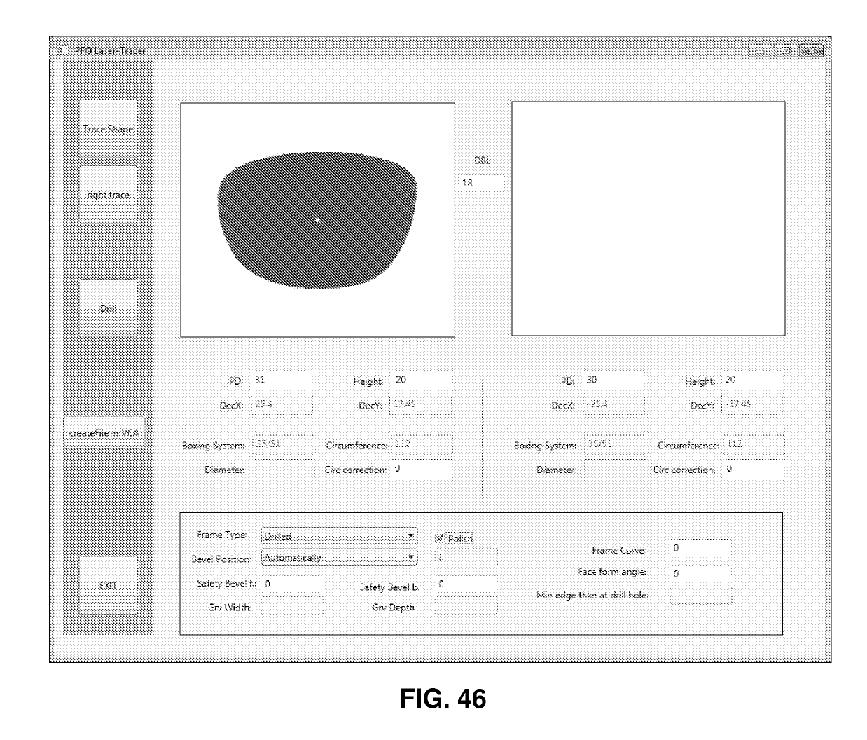

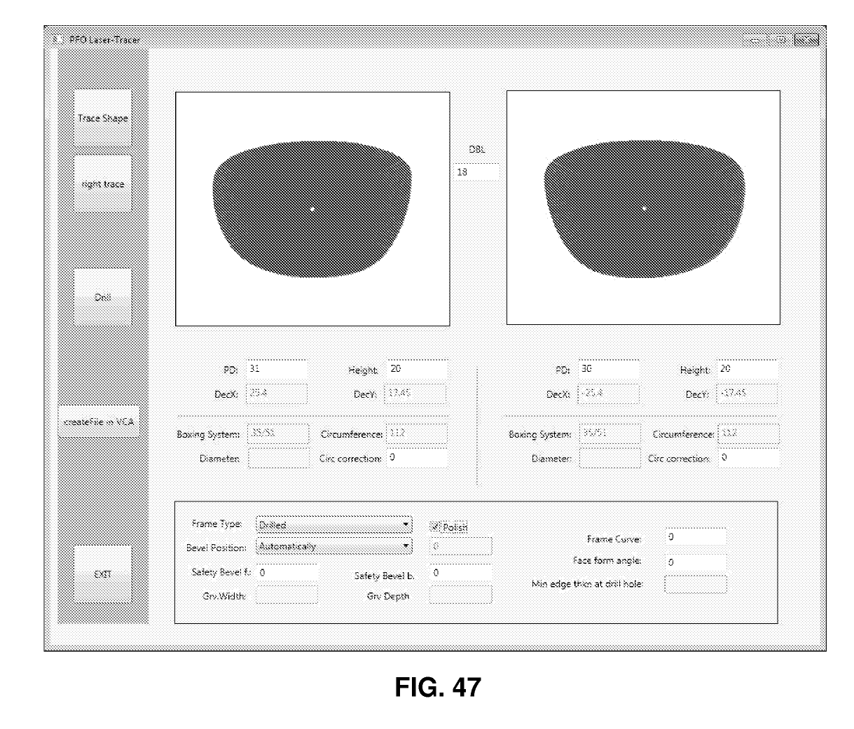

FIGS. 40 to 47 show various and exemplary user interfaces that can be employed to create the measurement data output by the tracers described herein. The user interface is provided for the ease of operating the inventive laser tracer to open or export trace data, to scan a frame, to scan a lens, to set up a profile, and/or to set up Internet, WiFi, etc. The user interface may be changed to display icons for ease of use in any format so that, in the end, it is user friendly. For example, FIGS. 40 to 42 show a user interface that displays the shape of each lens in an eyewear frame. FIG. 41 shows the shape of the left lens after being scanned and FIG. 42 shows the shape of the right lens after being scanned. In this exemplary embodiment, drill holes in the lenses are needed for attaching the lenses to the respective frame. FIGS. 43 and 44 illustrate an exemplary user interface that displays the contours of each lens (left lens in FIG. 43 and right lens in FIG. 44) and the locations where drill processing should occur on each lens. When that processing has completed, the two results are superimposed and are displayed in the user interface shown, for example, in FIGS. 45 to 47. FIG. 45 illustrates the user interface without either lens shown and FIGS. 46 and 47 respectively, show the left lens with drill holes and the right lens with drill holes. Once all of this data is collected and confirmed by the technician, the user interfaces can be employed to generate the lens-cutting data in the VCA format shown, for example, in FIG. 48.

FIGS. 49 through 73 illustrate the visual capture of an exemplary eyewear frame having clear blank lenses with an inventive camera from one side of the frame to the opposite side of the frame.

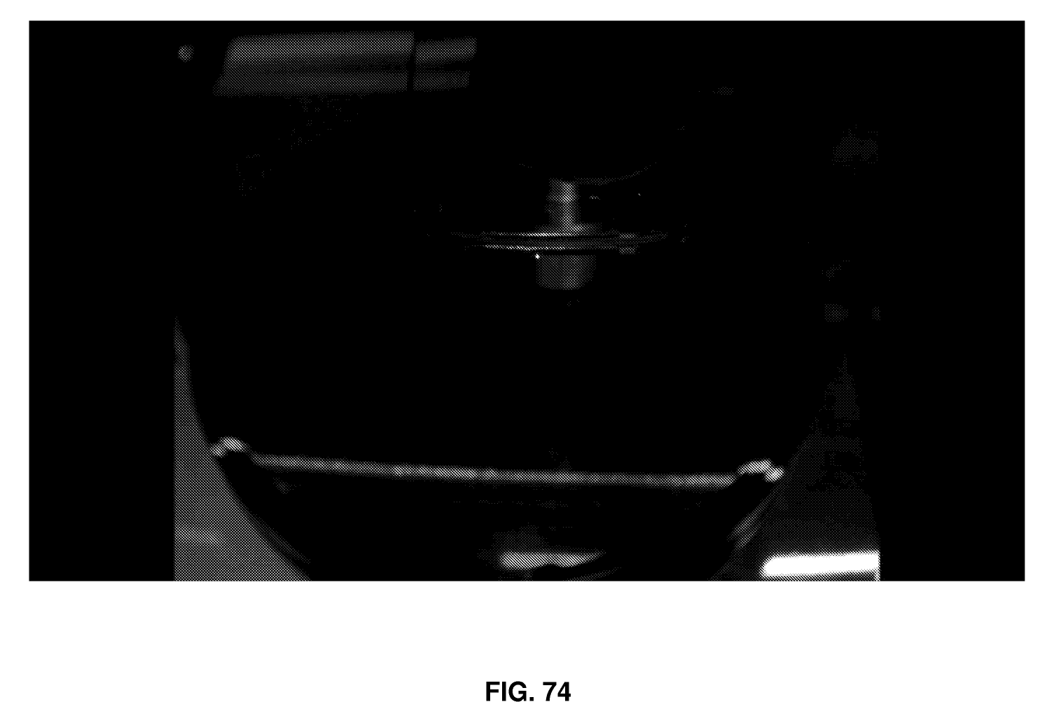

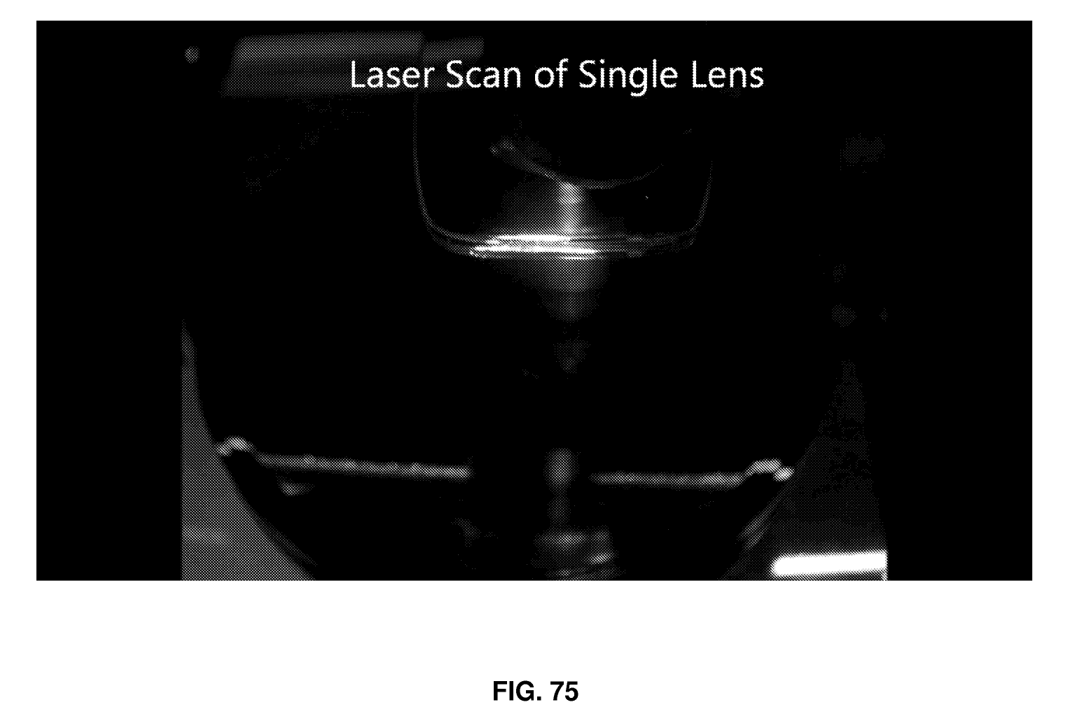

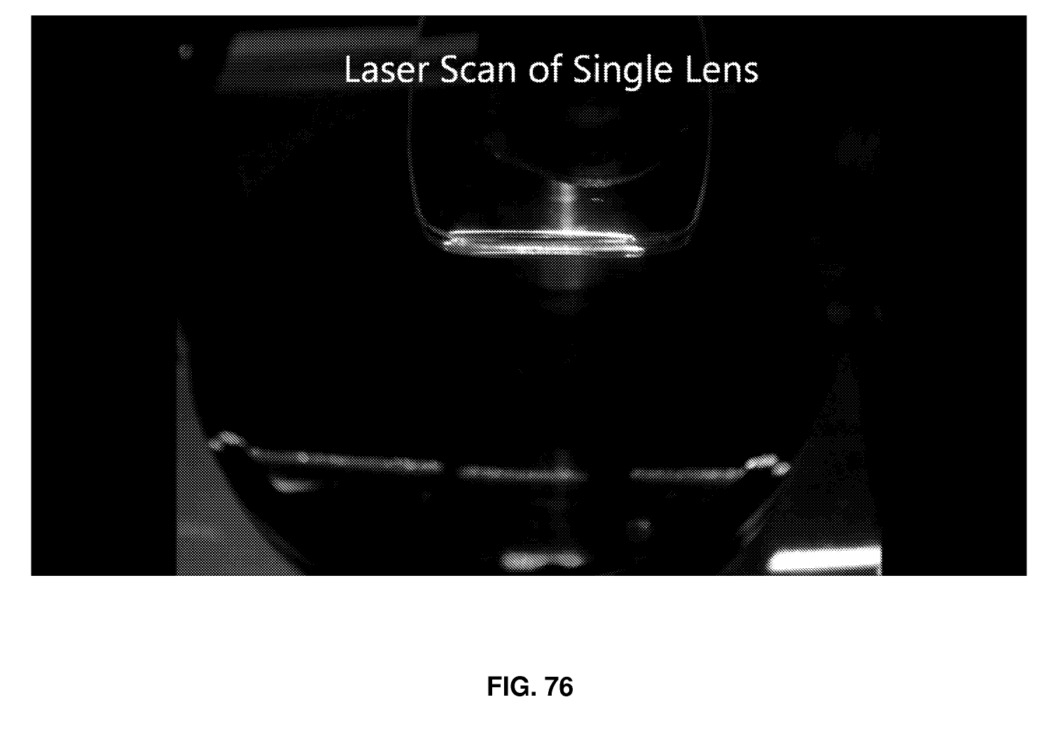

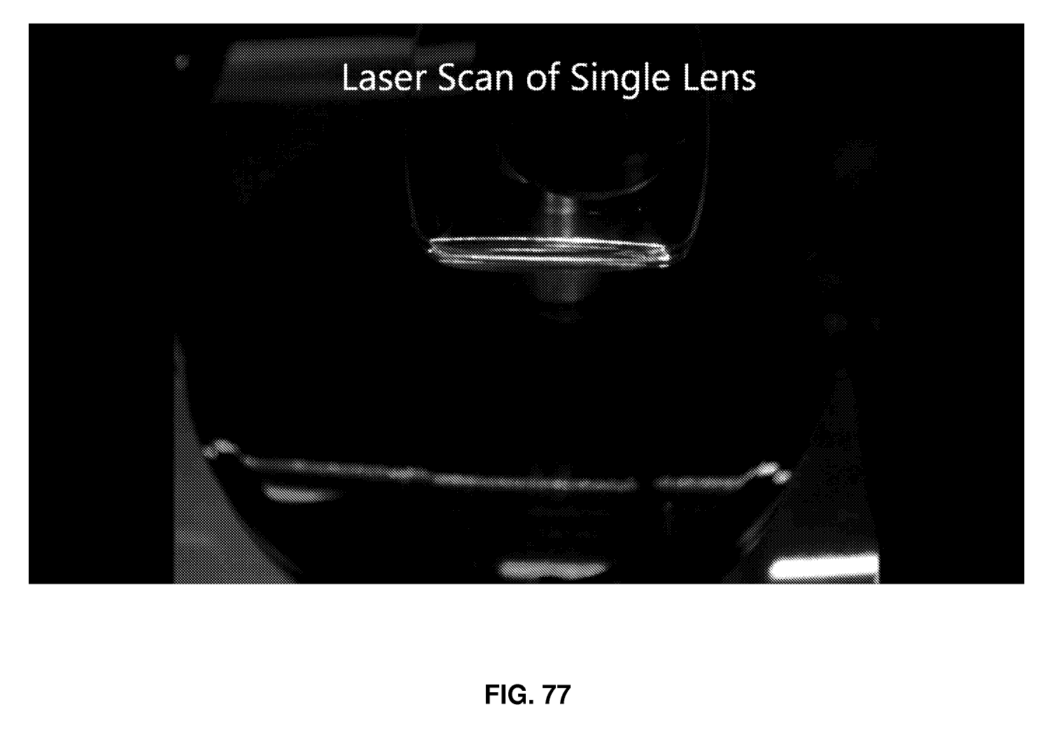

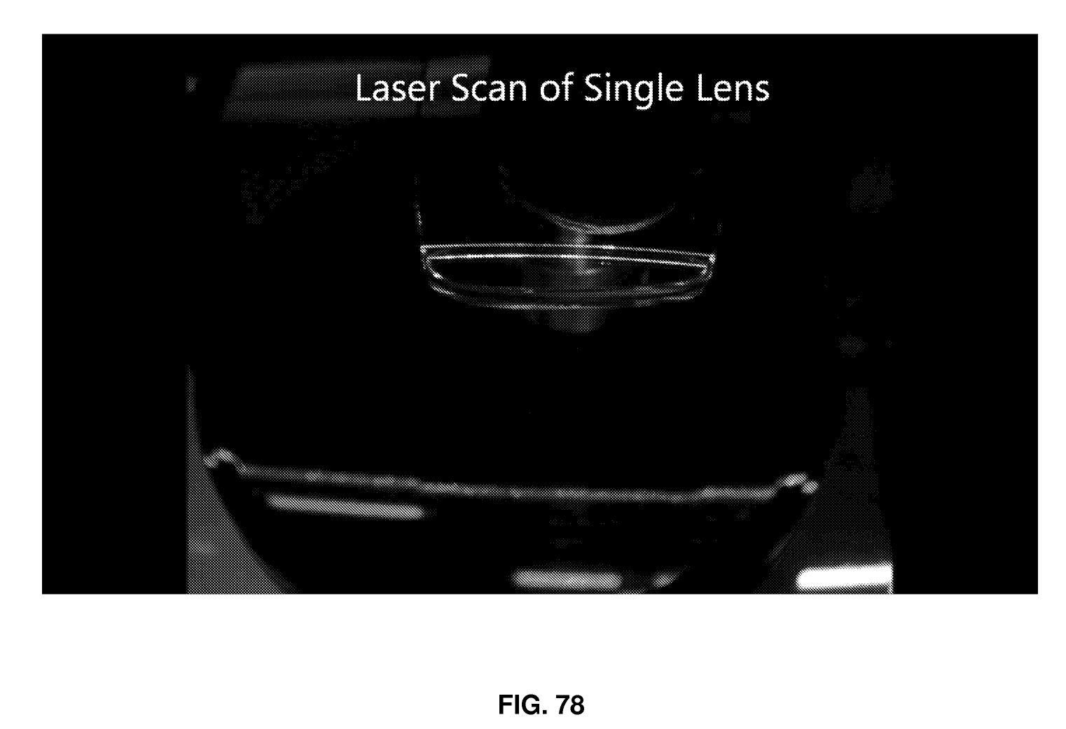

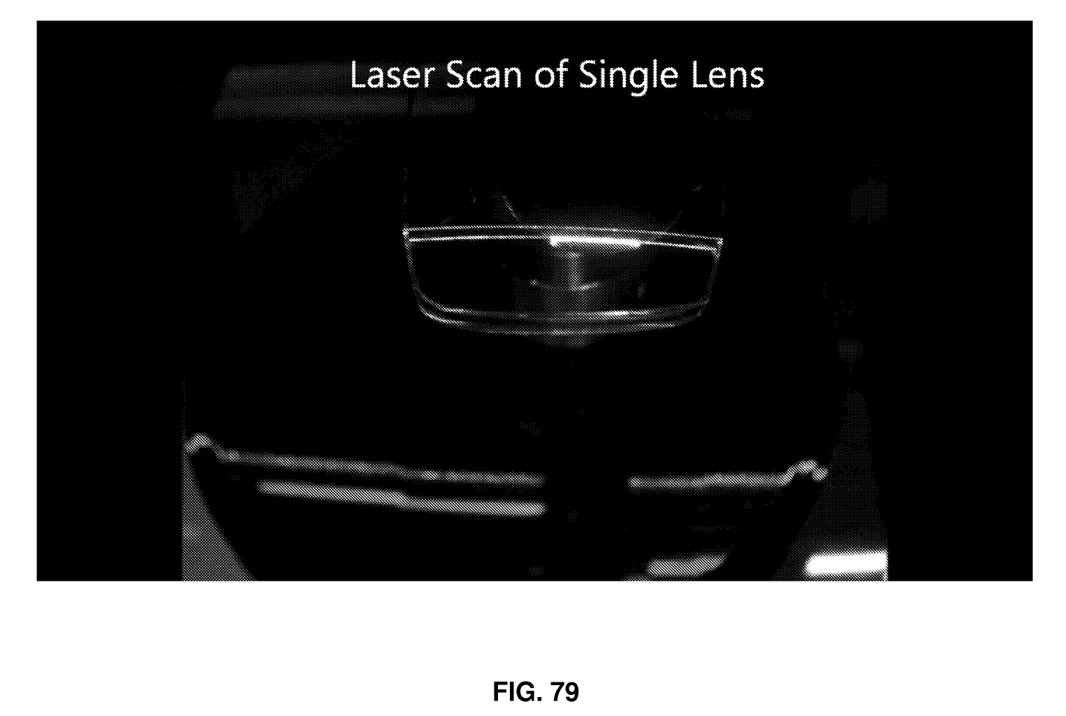

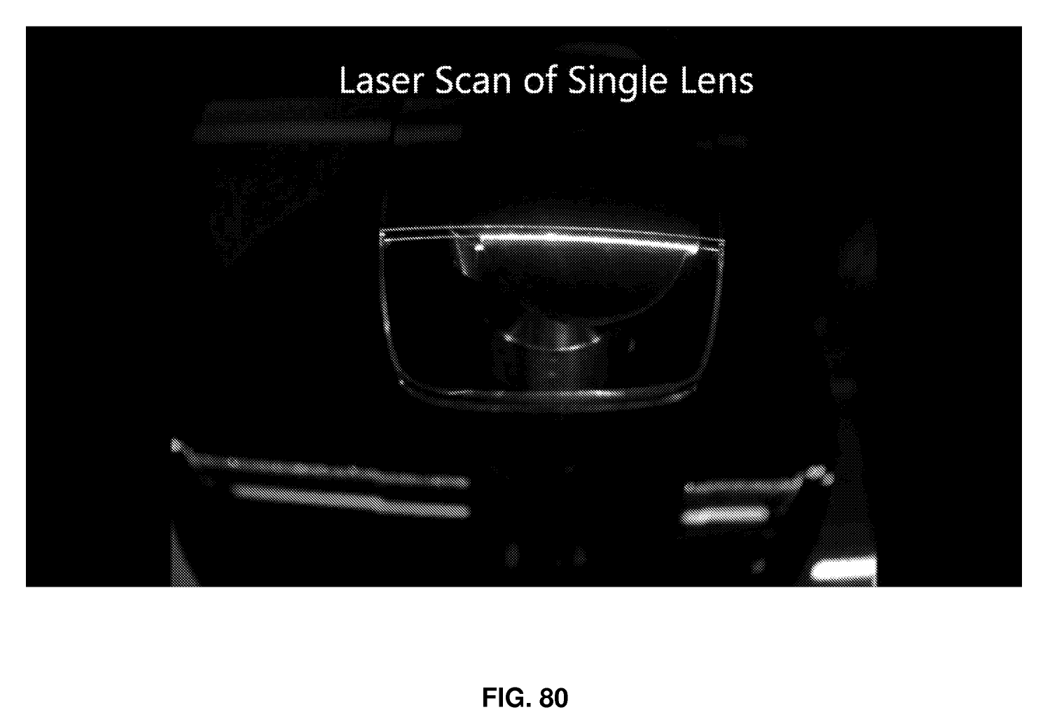

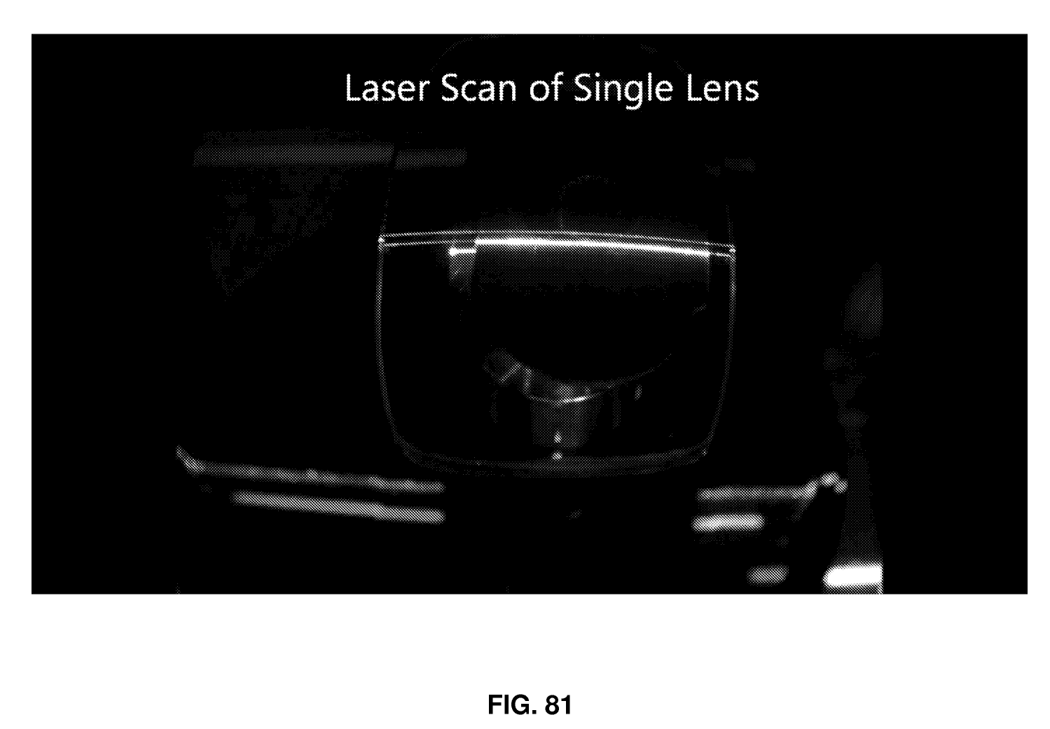

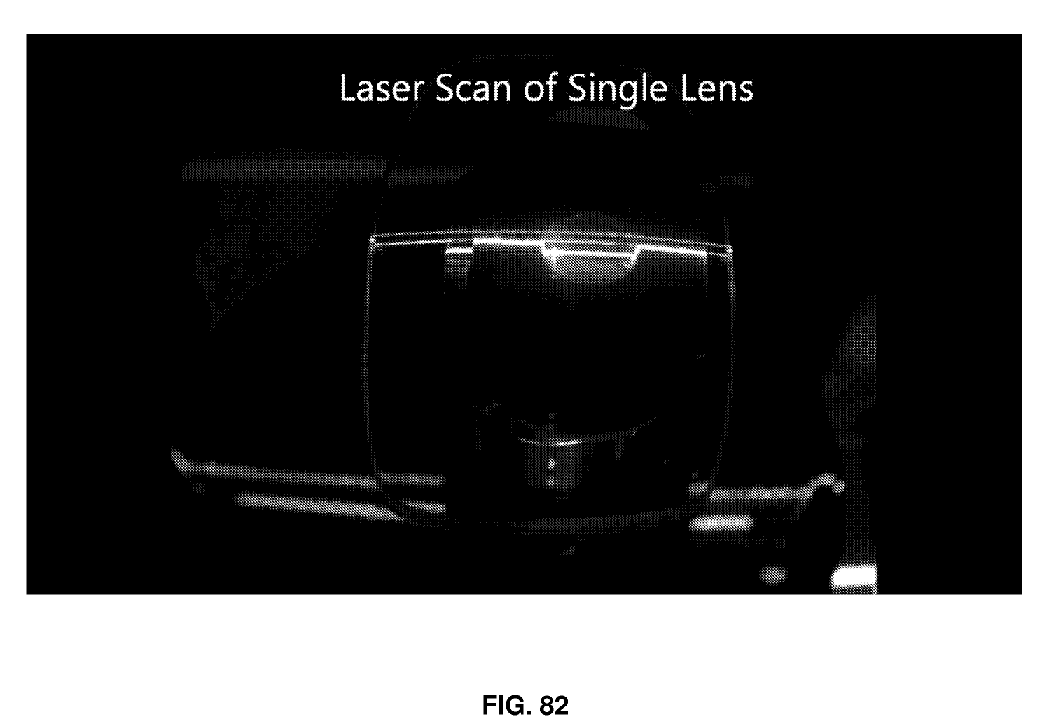

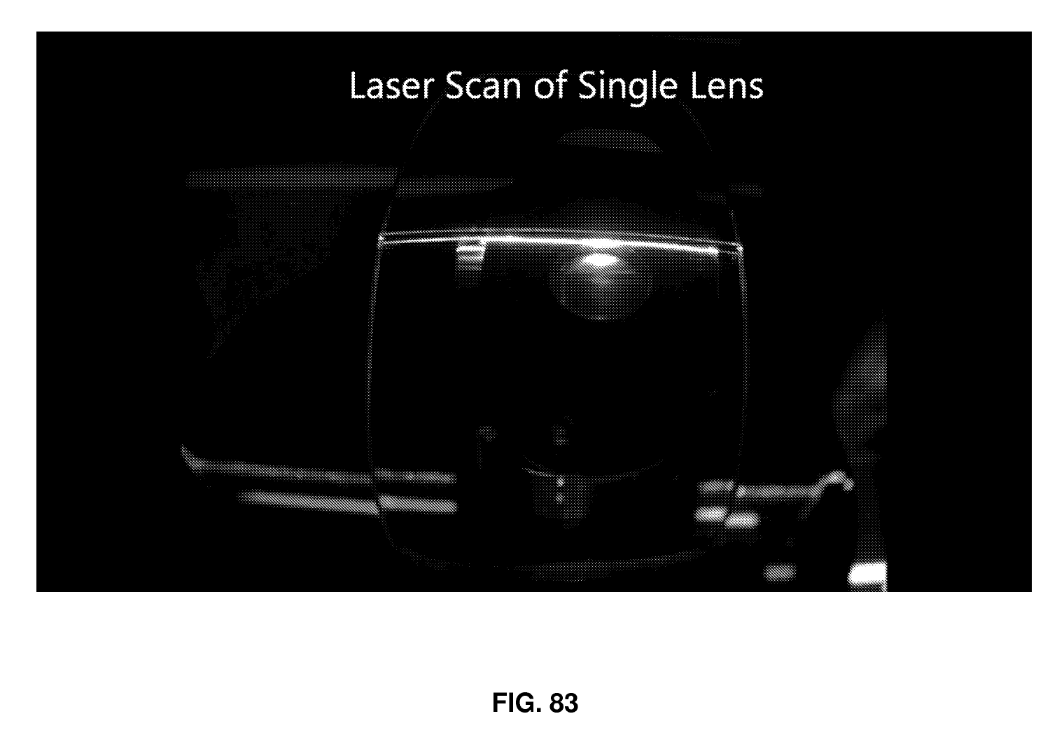

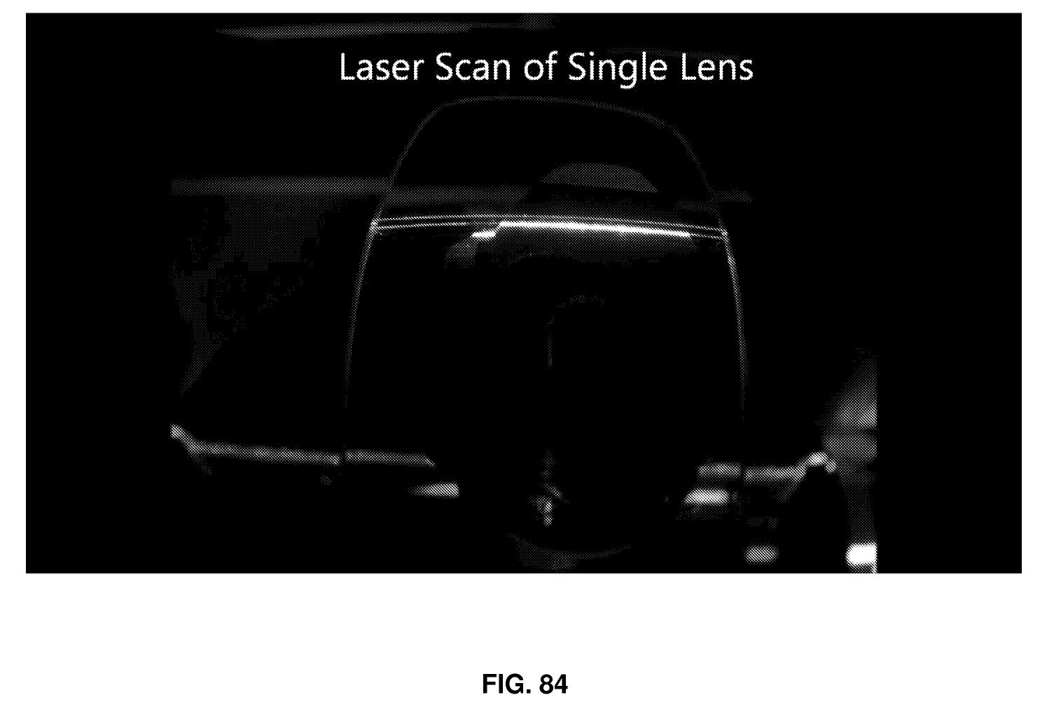

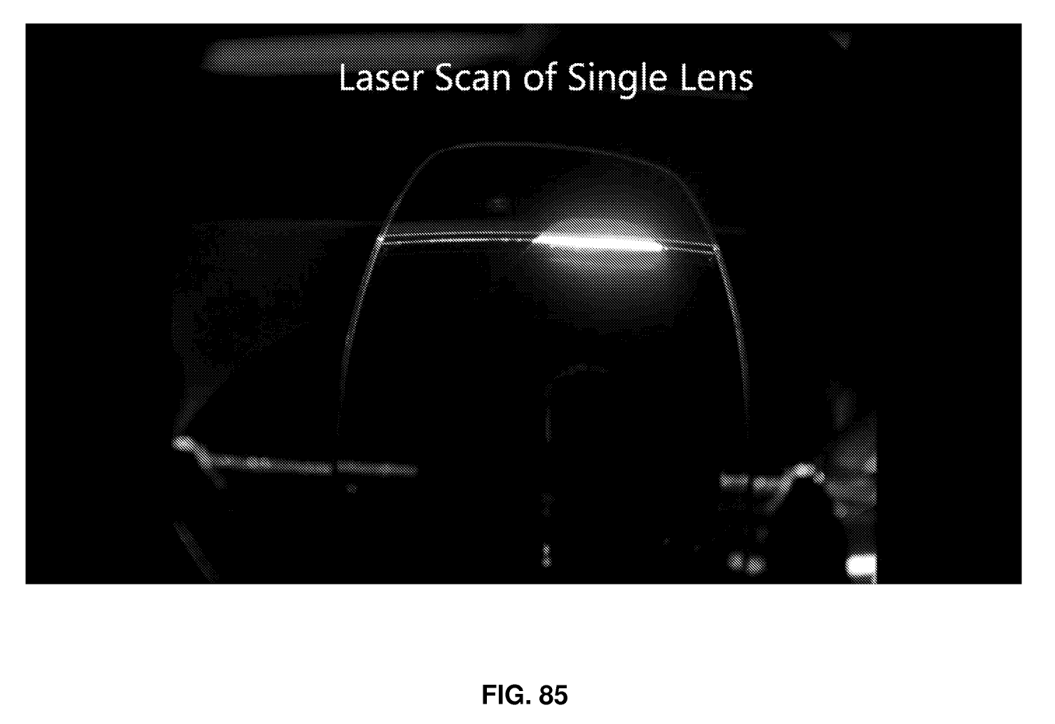

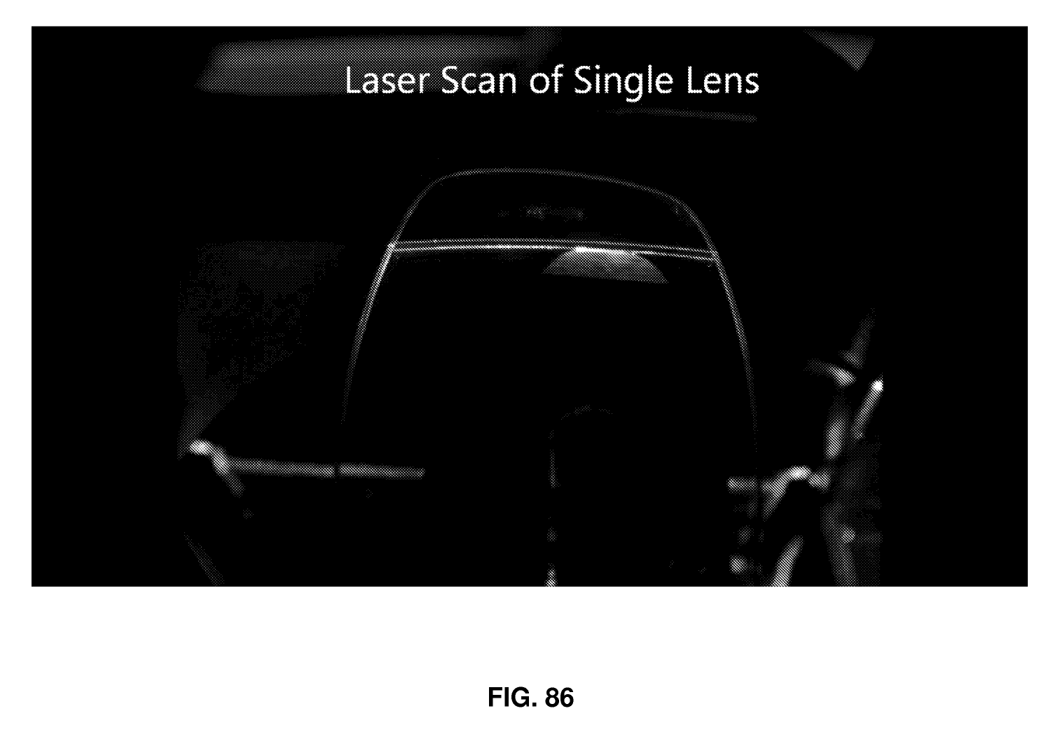

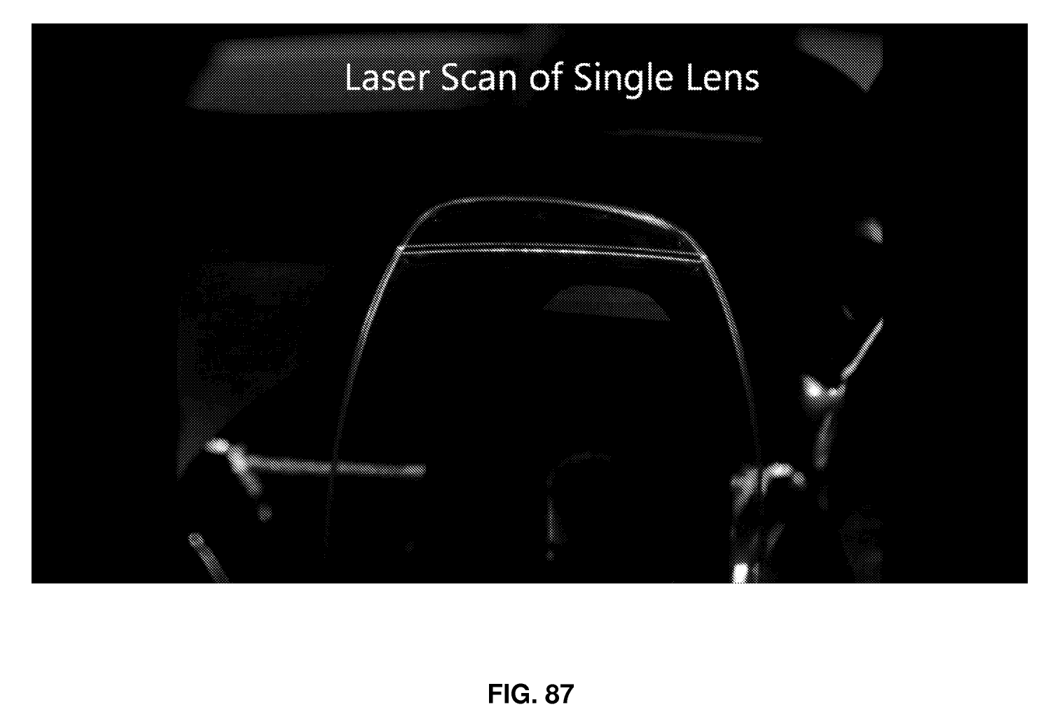

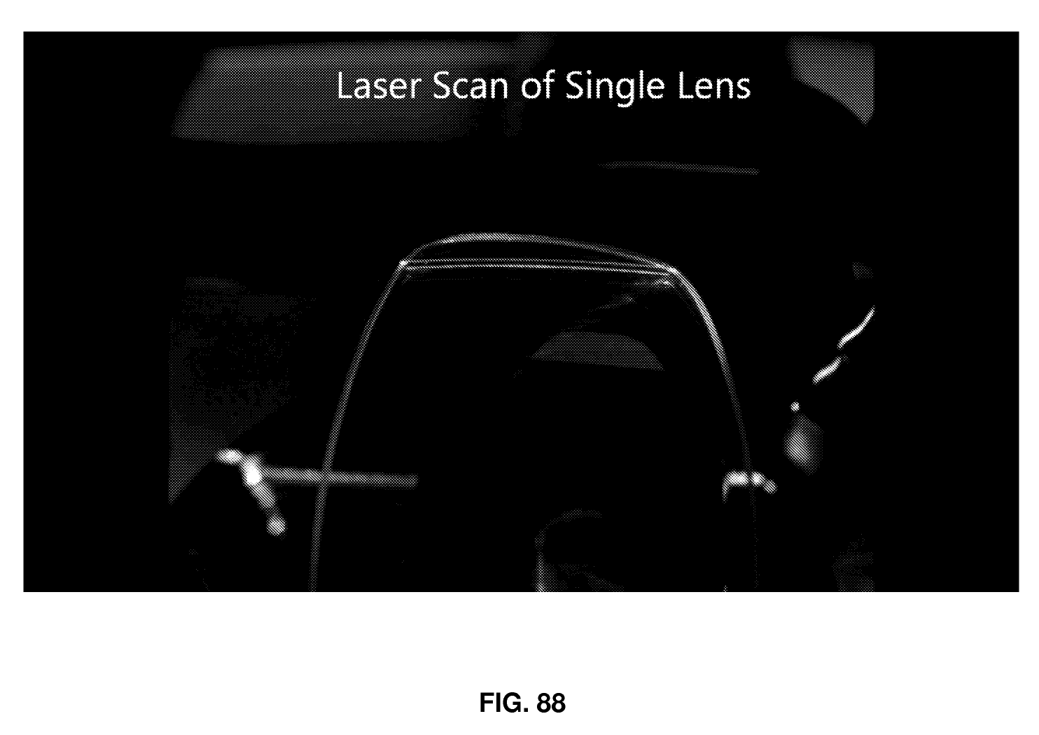

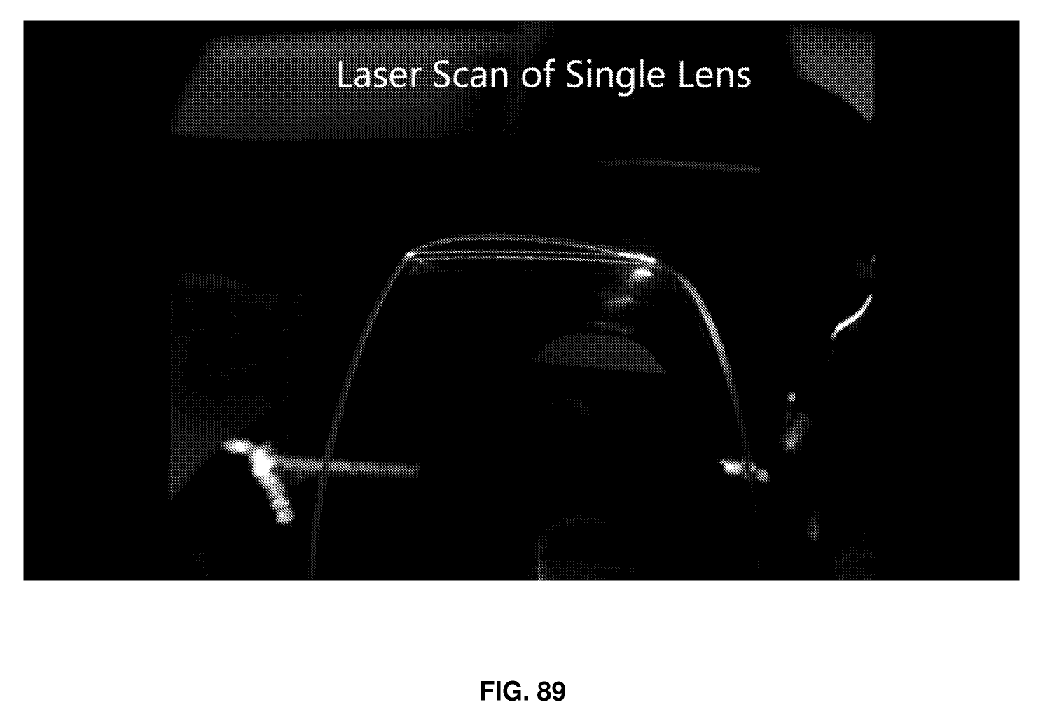

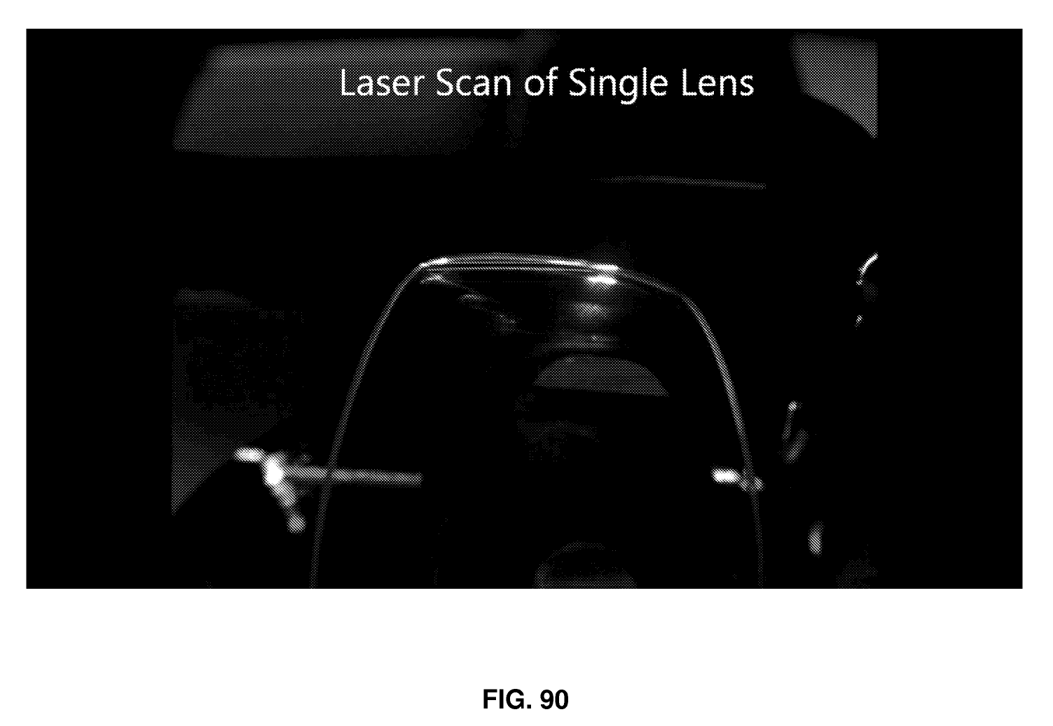

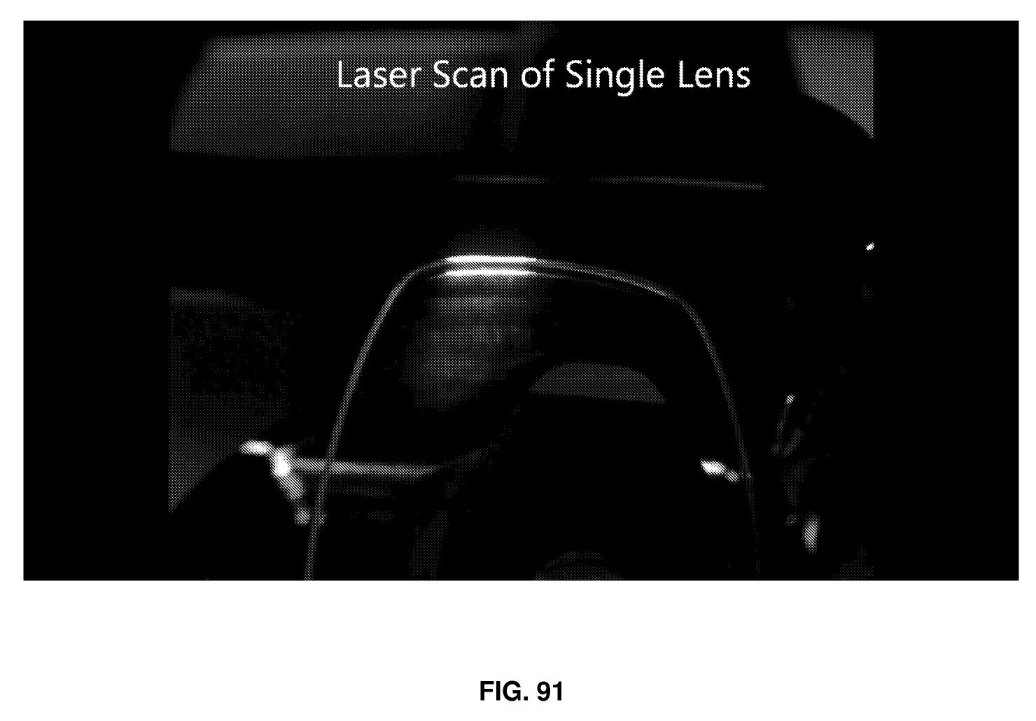

FIGS. 74 through 91 illustrate the visual capture of an exemplary eyewear lens with an inventive camera from one side of the lens to the opposite side of the lens.

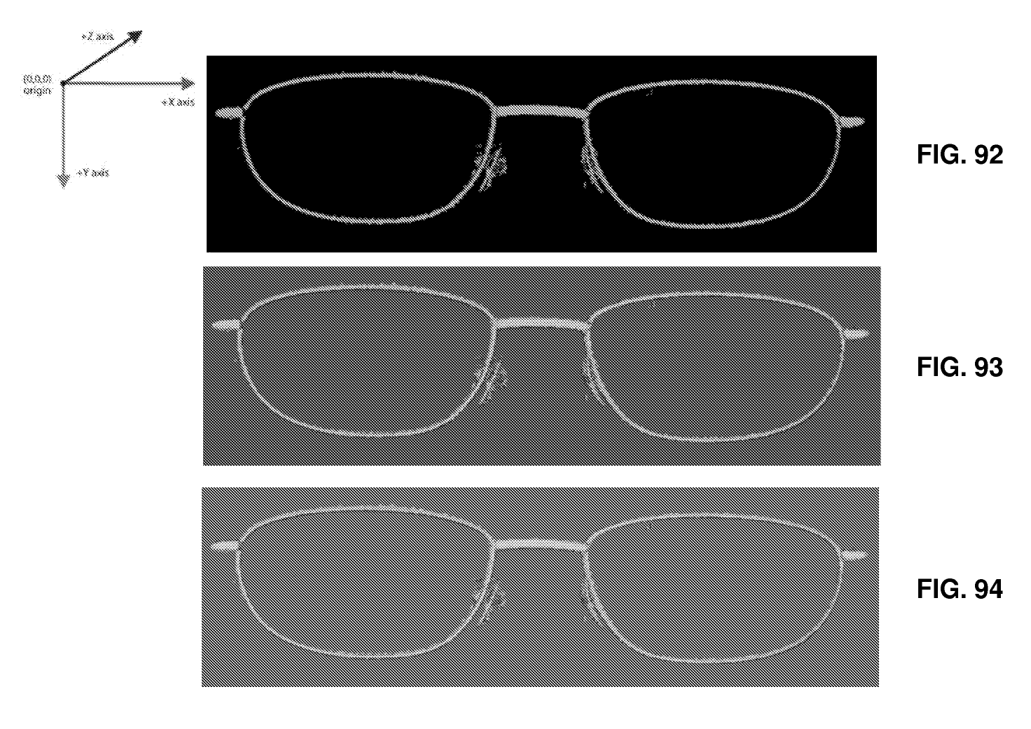

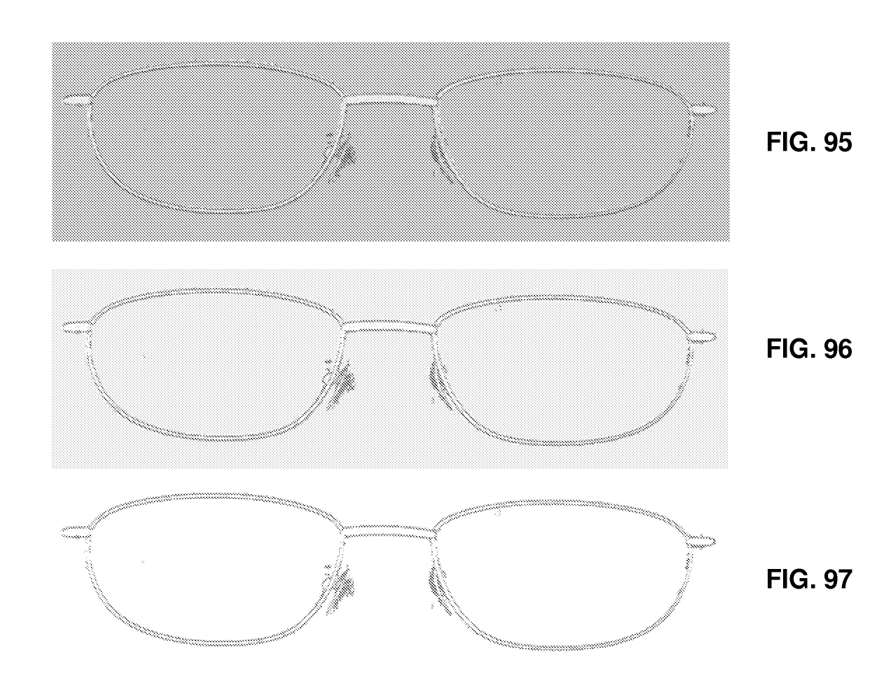

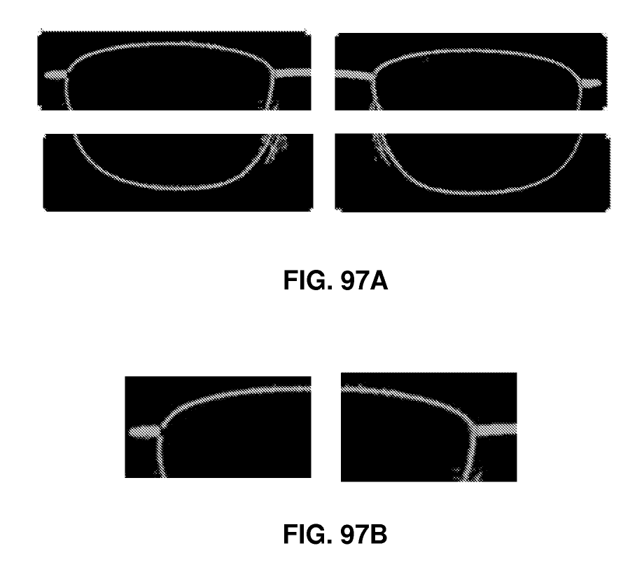

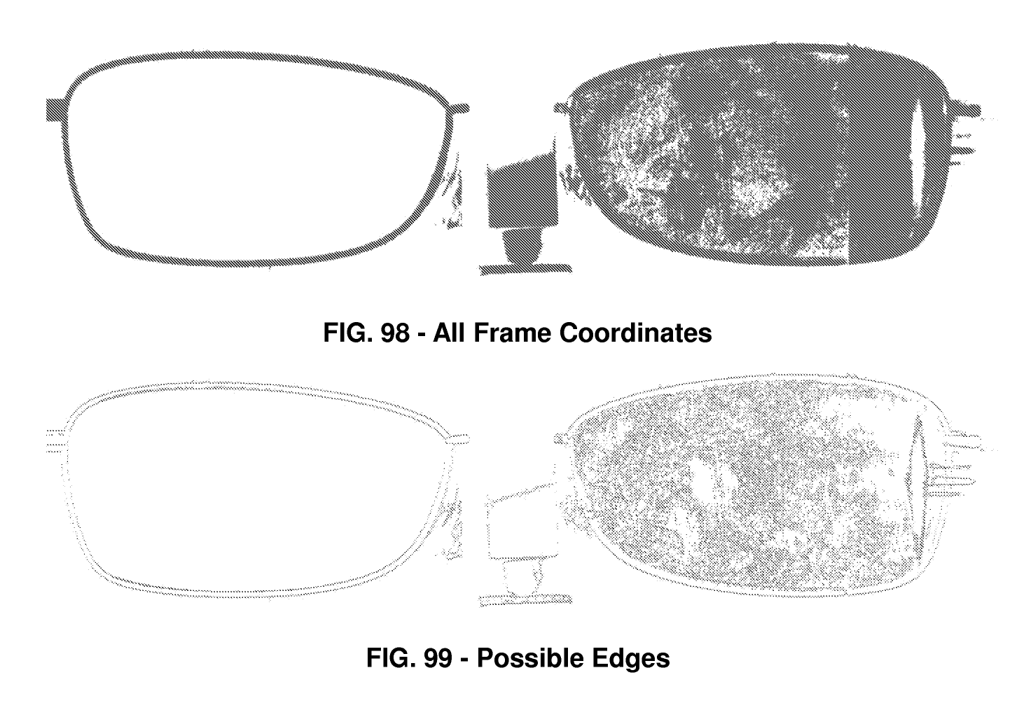

FIGS. 92 to 97 expand upon steps 395, 396, and 397 in FIG. 39 and illustrate various steps in the data processing algorithm to transform the picture visually captured by the camera into an accurate rendering of the eyewear frame having clear lenses. FIG. 98 shows all possible frame coordinates for the eyewear frame of FIGS. 92 to 97 and FIG. 99 show all possible frame edges for the eyewear frame of FIGS. 92 to 97 that, when processed, is able to output the scan result of FIG. 97.

In particular, once a 3D frame trace is captured from the laser tracer, the green data in the image of FIG. 92 contains x, y and z values. A first step of the processing is extracting 3D coordinates from these XYZ values. This data set contains all points on the surface of the lens, reflections, and some noise. Depending upon the type of scan, the program will get left-lens data or right-lens data. To do this, first, the min X and max X are identified and mid X is computed. From this, the scan data from min X to mid X comprises the left-lens data and the scan data from mid X to max X comprises the right lens.

A second step involves cleaning out invalid scan data with signal processing (e.g., Mode). In this cleaning process, all Z values of Y are scanned for a given X (bottom to top/vertically traverse Z) and a mode is determined, referred to as a Scan-wide Mode Filter. In summary, based on this mode, all points lying too far from the mode value (too high or too low) are removed. In particular, the Mode is computed from all of the Z values from the scan. Then, all scan data outside the range of {Mode−A and Mode+2A, where A is a constant} is discarded. The scan is rotated to obtain the maximum width. To make this determination, in step 1, angular increments are determined by rotating the scan along the X, Y, and Z axes. A rotation matrix is computed in step 2. For each point in the scan, in step 3 the rotation matrix is applied to compute a new transposed location. Then, in step 4, width and height of the new scan is computed. Steps 1 to 3 are repeated for 15 degrees and the resulting values are compared to the values computed in Step 4. The point at which the maximum width and height are observed is the point at which the scan object is orthogonal to the laser. In a third step, all isolated points are removed from the scan. To do this, the algorithm traverses through each point and counts the number of neighbors.

A minimum neighbor count(m) and a neighborhood threshold(n) is determined. For each point in the scan (i,j), the algorithm traverses the neighbors { i−n, j−n} to { i+n, j+n}. For each non zero Z(i,j), the value of c is incremented. At the end of the traversal, if c≧m, then the 3D coordinate is copied to a valid point array and the point is not copied to valid point array if c<m.

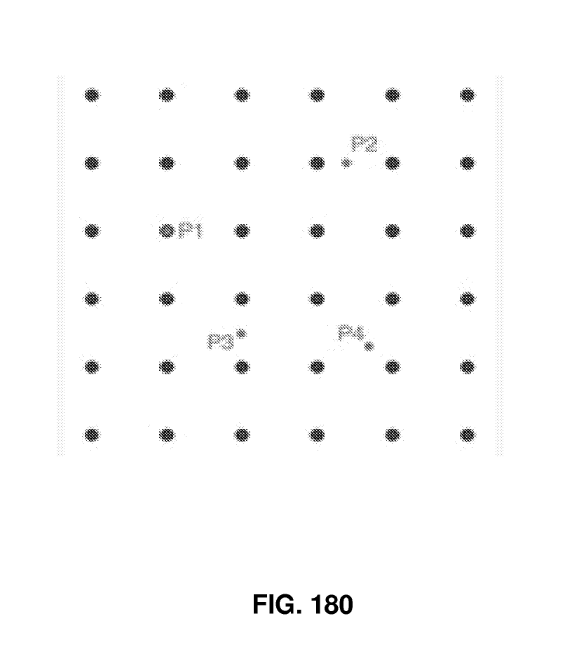

FIG. 180 illustrates such neighbor counting. If the number of neighbors does not meet a threshold value, the point is considered invalid and is removed from the scan set. Then, a mode filter for Y is conducted. In particular, all (x,z) are extracted from each y in the scan. The Z mode (M) is computed at each y. Finally, points that are outside the range of {M−A and M+A} are removed.

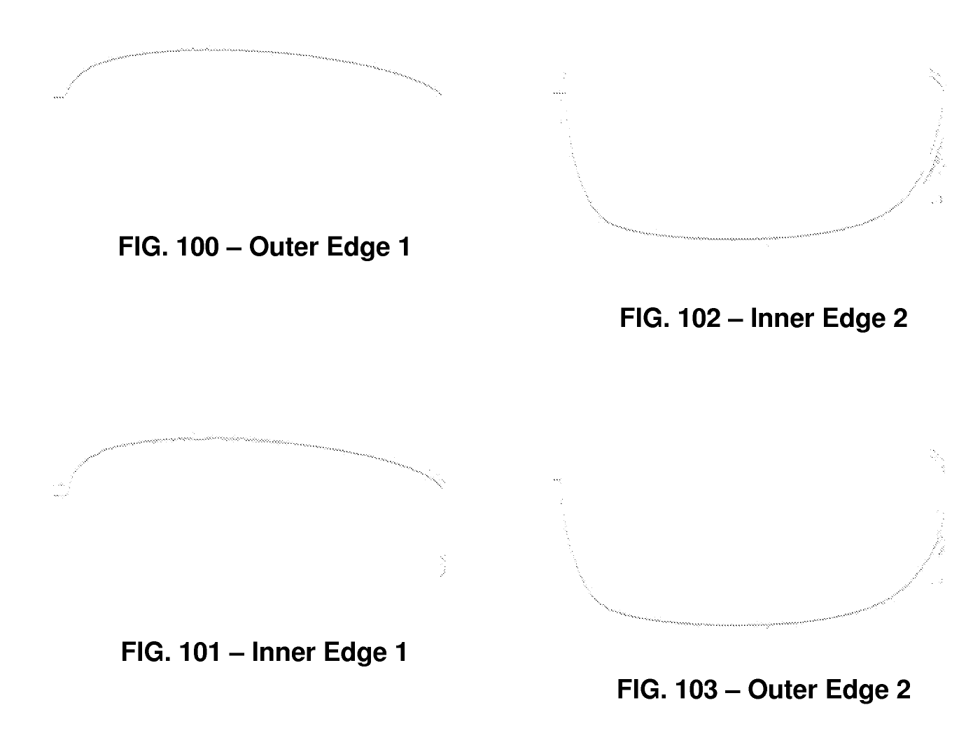

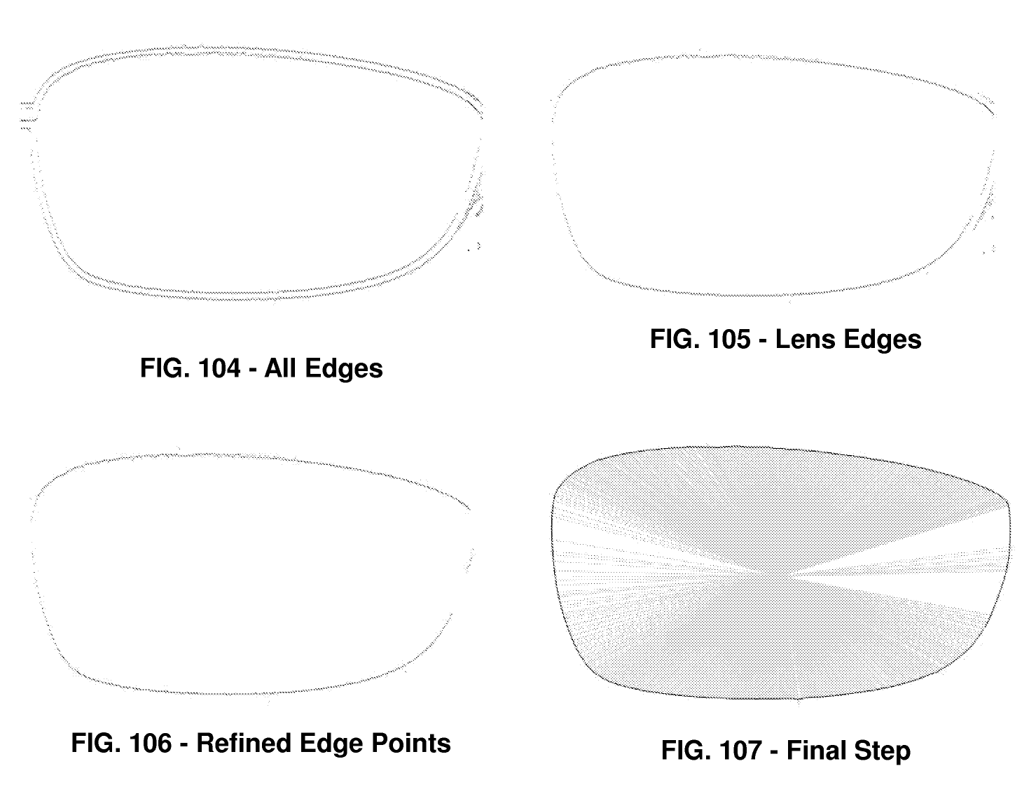

Edge detection occurs in a fourth step. At this point, most of the noise and the invalid data have been removed and the data quality is good enough to proceed for edge detection. First, the inner edges are extracted. To do this, the scan is divided into four parts by identifying the min x, the min y, the Max X, and the Max Y, which division is shown, for example, in FIG. 97A. Each of these sets is further divided into two parts, as shown in FIG. 97B for the upper left quadrant of FIG. 97A. In each of these eight sets, the algorithm traverses vertically and horizontally to identify a first point with a non-zero Z value and a last point with a non-zero Z value. These points are defined as the edges of the frame. In particular, starting from mid X and mid Y, the nearest point at each x, y is computed. From this, it is determined that all of the nearest points to the center are the possible inner edge of the frame. A first outer edge determined in this way is illustrated in FIG. 100. A first inner edge determined in this way is illustrated in FIG. 101. A second outer edge determined in this way is illustrated in FIG. 102. Finally, a second inner edge determined in this way is illustrated in FIG. 103. All of the edges are shown together in FIG. 104. From this, the lens edges can be determined and are shown in FIG. 105.

After the edges are identified, any isolated points are removed and valid points are merged. In particular, the points are reordered and filtered by radius. The inner edges extracted from the above step are extracted to spherical coordinates. The coordinates are rearranged by θ. The algorithm traverses through (r, θ, φ) and filter all points which do not satisfy |r(i)−r(i−1)|<e.

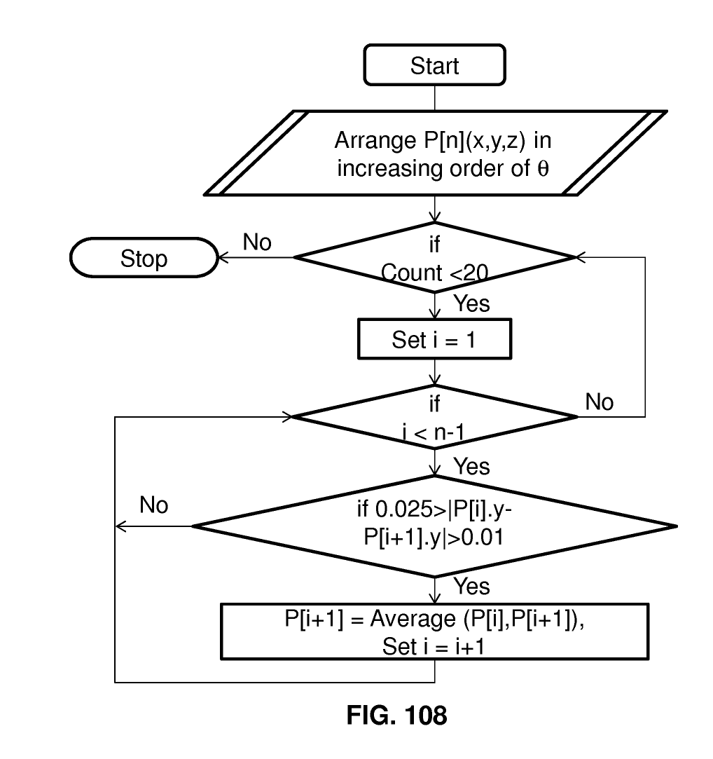

These merged valid points forming edges are smoothened, which can be performed by employing one or more curve fitting algorithms and is shown in FIG. 106. In particular, the radius of immediate neighbors can be averaged to achieve a curve fit by applying a smoothening and average Fit curve fitting algorithm. This smoothening algorithm/curve fitting algorithm takes into account the observation that the edges are noisier in the direction of the linear travel (Y-Axis) (left and right edges). All of the edge coordinates are sorted in an angularly increasing order, 0 to 360 degrees as illustrated in the flow chart of FIG. 108. If the difference between the neighboring points A,B is <0.025 mm and >0.01 mm in the Y coordinate, substitute (x,y,z) for B with an average of A and B. If the difference between the neighboring points A,B is <0.01 mm in the Y coordinate, this means that the points are close enough to follow a smooth curve and smoothening is not applied. If the difference between the neighboring points A,B is >0.025 mm in the Y coordinate, the possibility is that the algorithm is traversing through the edges of the frame where the variation in Y is expected (top or bottom edge) and no smoothening is applied. By iterating through this loop 20 times for all of the edge points, a smooth curve closest to the observed points is achieved. Similarly, algorithms such as Bezier, Gaussian, B-Spline, and/or Non-Uniform Rational Basis Spline (NURBS) can be utilized. A final result can be achieved that is shown, for example, in FIG. 97 and FIG. 107.

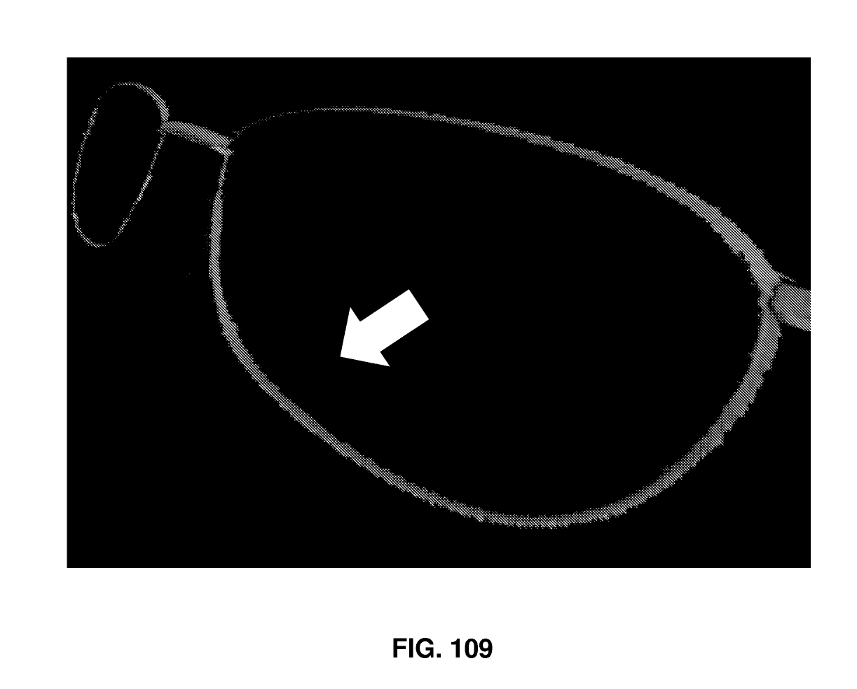

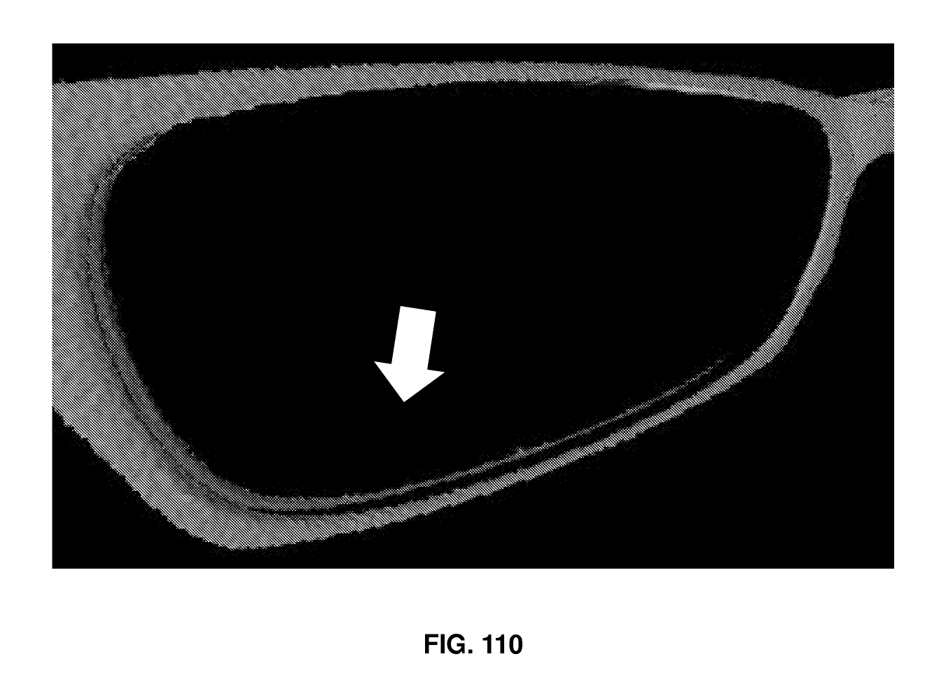

Capturing data from the eyewear frame substantially orthogonal to the frame does not necessarily obtain data on the bevel of the interior of the lens opening. Accordingly, the tracer obtains laser-camera data at an angle to the front face of the frame, which results are shown in the examples of FIGS. 109 and 110. In particular, the view of FIG. 109 is from above in front of the left lens opening and, in this orientation, it is possible to obtain bevel characteristics at least from the inside lower corner of the left lens opening, indicated with the arrow. With a view that is more at an angle to the orthogonal of the front of the frames, as shown in FIG. 110, the bevel (indicted by the arrow) can be even more clearly viewed and, accordingly, traced.

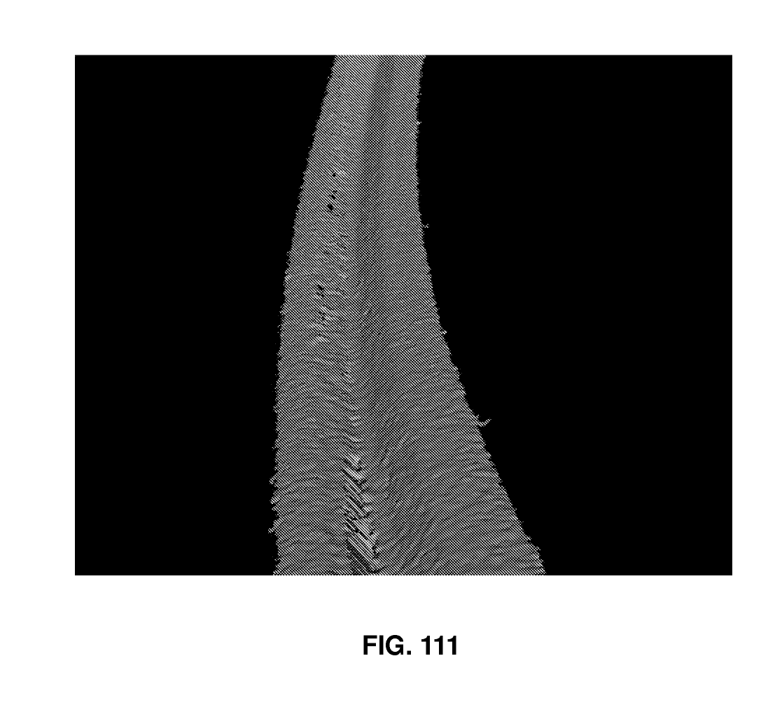

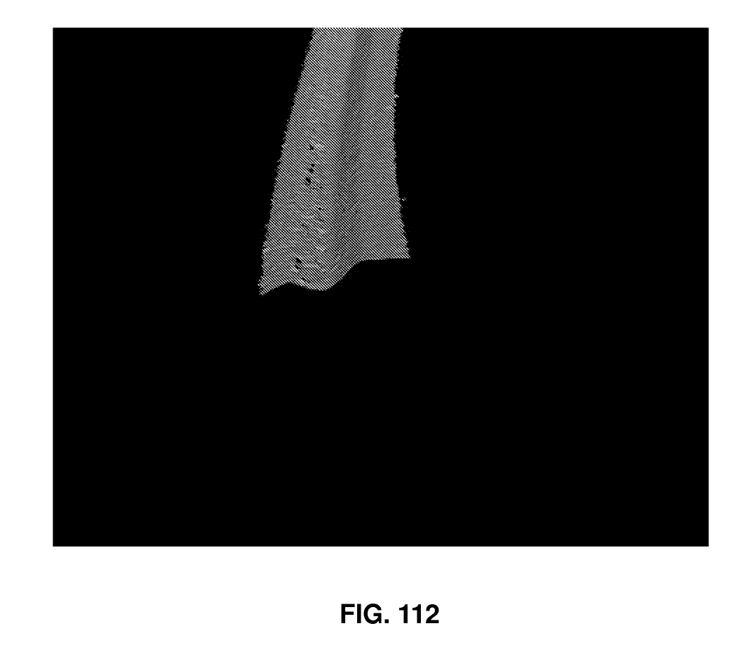

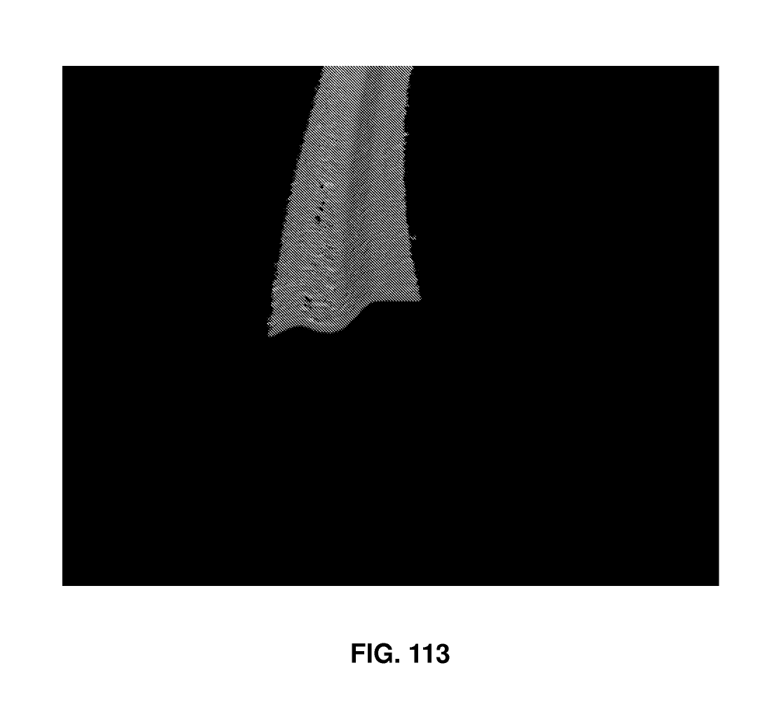

FIGS. 111 to 112 illustrate how a frame bevel is traced. During the 3D laser scanning, the tracer is able to form an accurate 3D depiction of at least a portion of the frame bevel, which is shown in FIG. 111. From this, it is known that an accurate edge bevel of a lens can be determined by taking a dissection of the 3D depiction of the frame bevel. Accordingly, through software of the tracer, the 3D laser scan is dissected, as shown in FIG. 112, and, in FIG. 113, the tracer creates a dissection contour. This dissection contour defines the edge bevel that is to be shaped on the outer edges of a lens to fit the respective lens opening of the scanned frame.

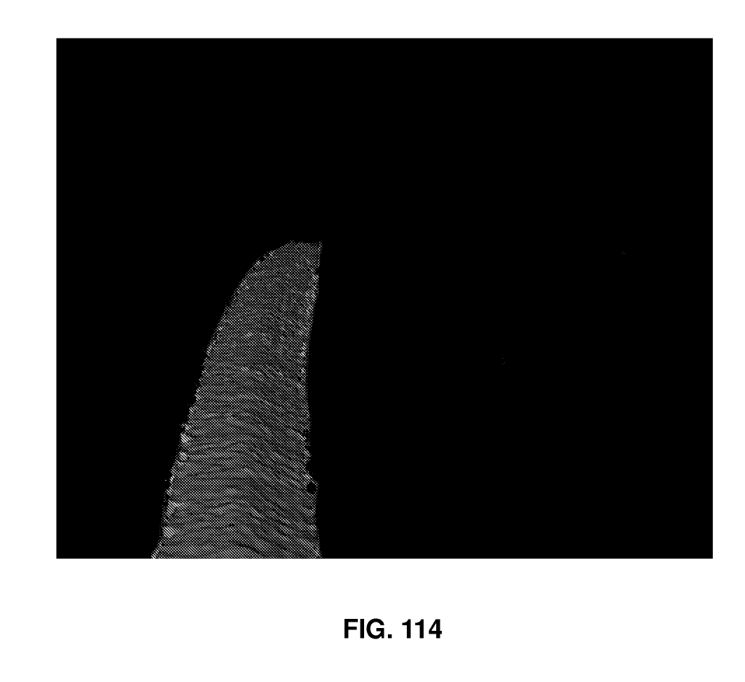

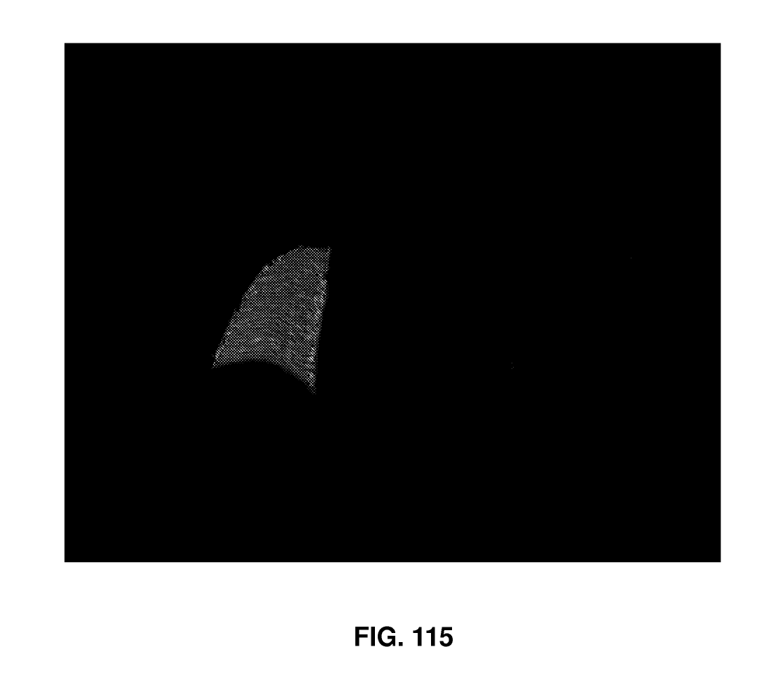

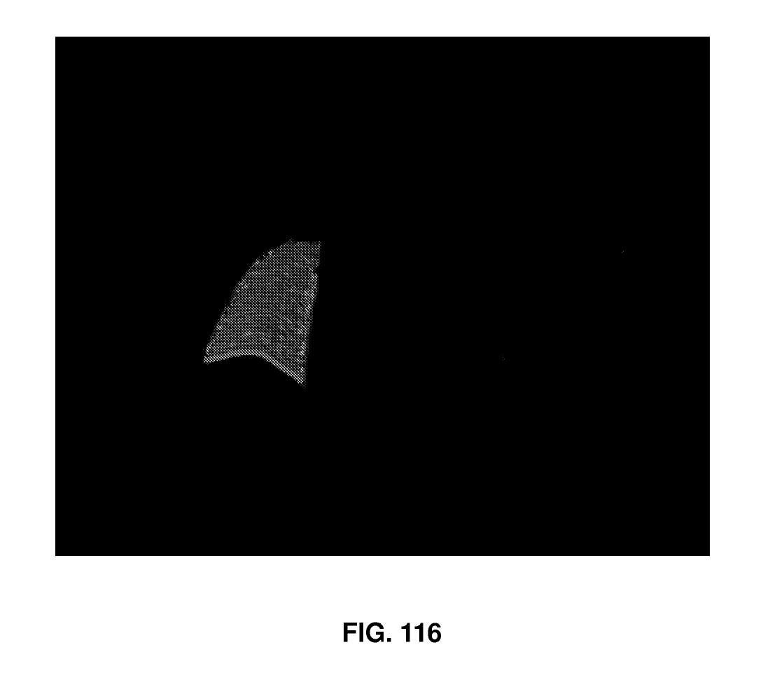

The same process can be used to determine and create an accurate lens bevel of provided lenses. This is shown with regard to FIGS. 114 to 116. First, the 3D laser eyewear tracer forms an accurate 3D depiction of at least a portion of the edge bevel, which is shown in FIG. 114. An accurate edge bevel for the remainder of the lens to be created can be determined by taking a dissection of the 3D depiction of this edge bevel. Accordingly, through software of the tracer, the 3D laser scan is dissected, as shown in FIG. 115, and, in FIG. 116, the tracer creates a dissection contour. This dissection contour defines the edge bevel that is to be shaped on the outer edges of a lens to fit the respective lens opening of the scanned frame.

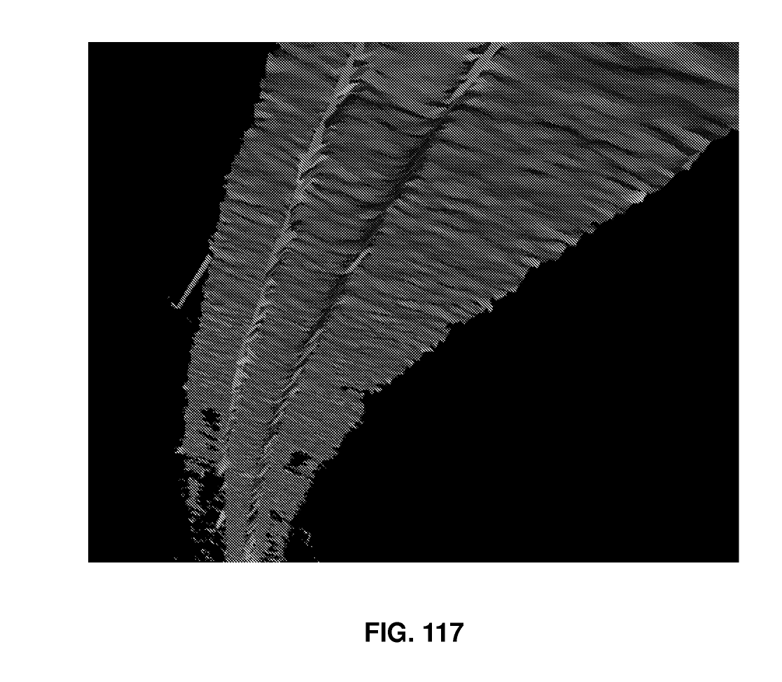

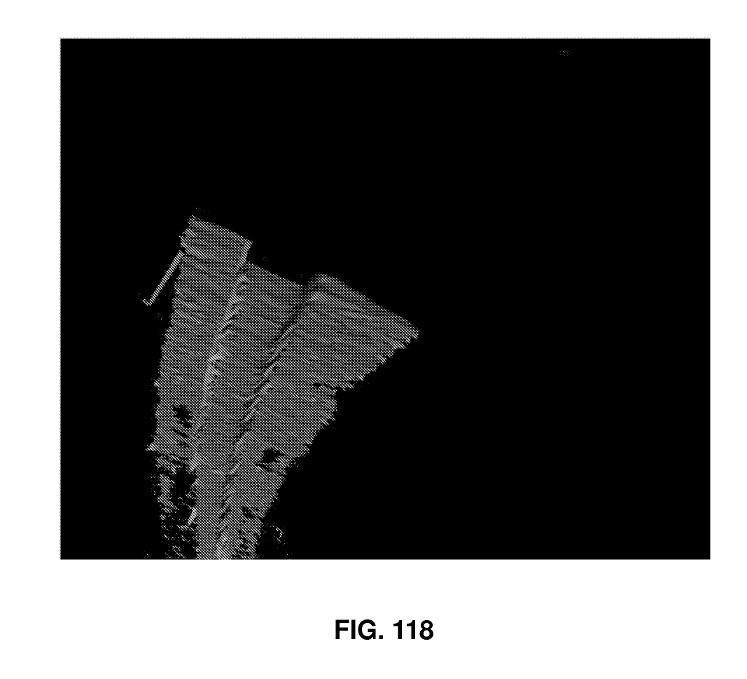

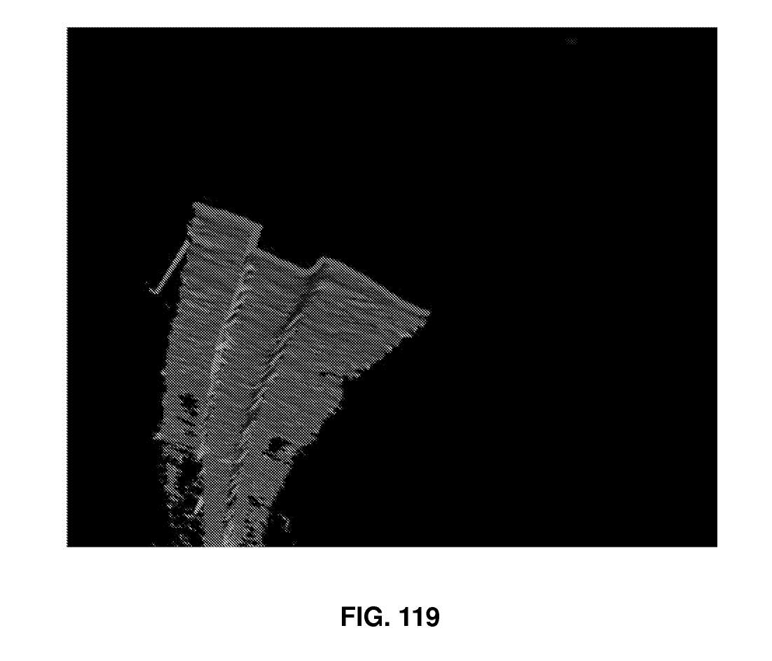

Some eyewear does not have frames around the lenses and, instead, each lens is held by a nylon or fish wire or cord that surrounds the lens and tightly holds it within a groove on the outer edge of the lens. This, too, can be accurately measured and reproduced by the 3D laser eyewear tracer as shown in FIGS. 117 to 119. First, the 3D laser eyewear tracer forms an accurate 3D depiction of at least a portion of the lens edge, which is shown in FIG. 117. An accurate lens edge for the remainder of the lens to be created can be determined by taking a dissection of the 3D depiction of this lens edge portion. Accordingly, through software of the tracer, the 3D laser scan is dissected, as shown in FIG. 118, and, in FIG. 119, the tracer creates a dissection contour. This dissection contour defines the lens edge that is to be shaped on the outer edge of a lens to fit the respective frame loop.

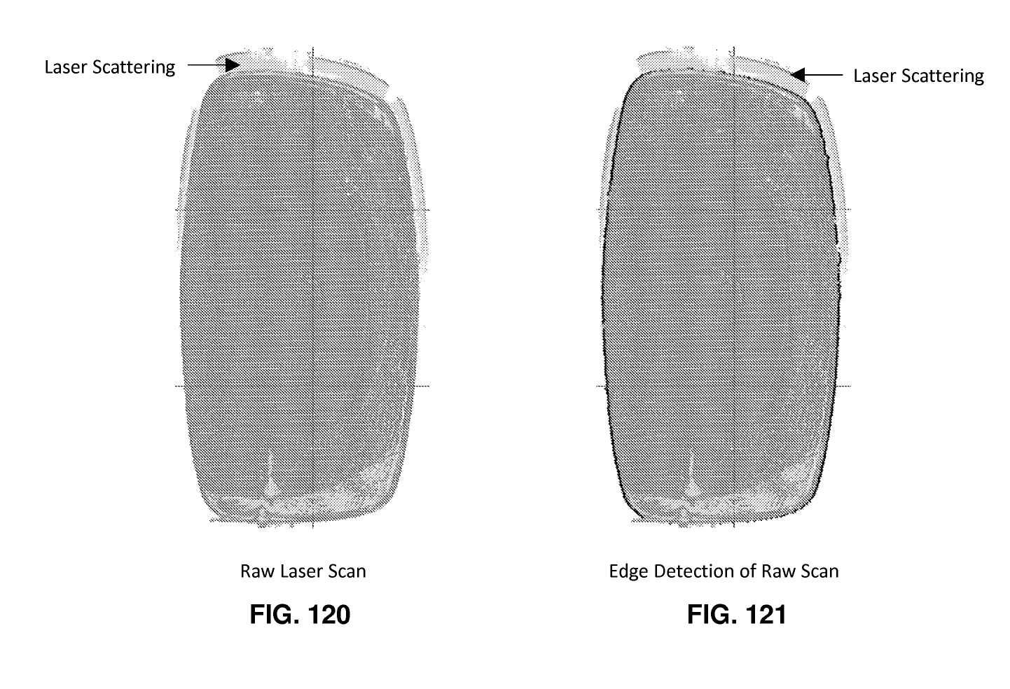

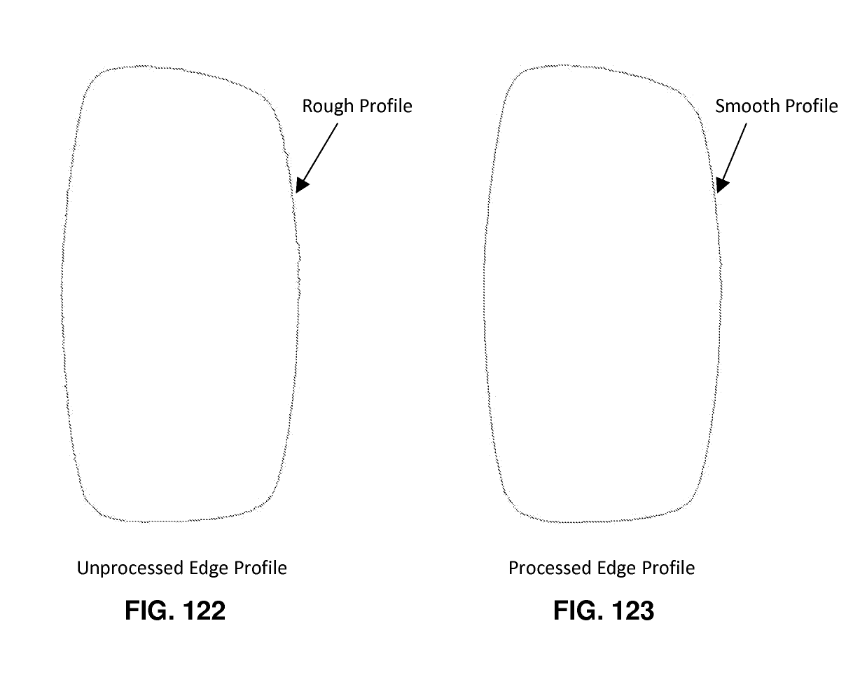

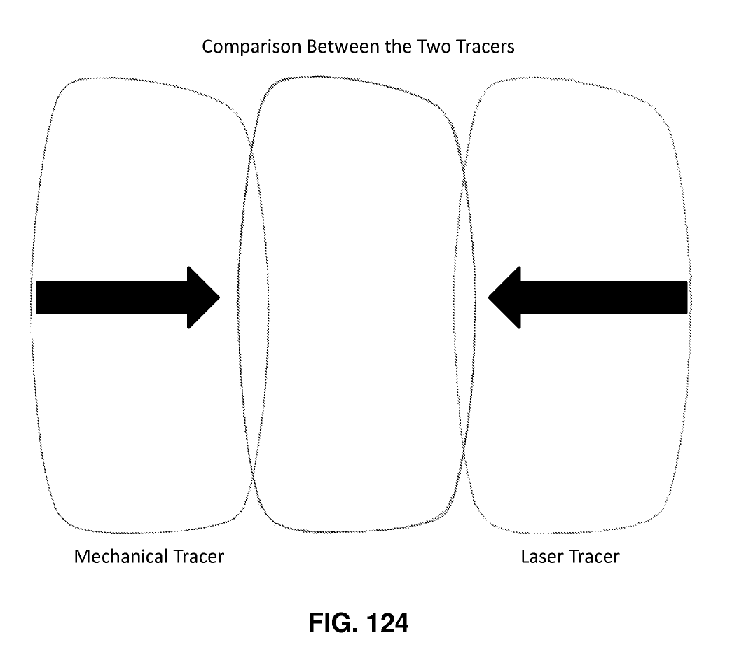

Other smoothing functions of the 3D laser eyewear tracer are described with regard to FIGS. 120 to 124 and show how the 3D laser eyewear tracer is equal to or better than prior art mechanical tracers. First, the 3D laser eyewear tracer performs a raw scan of a single lens, which is depicted in FIG. 120. The raw scan includes laser scattering that is to be removed by the 3D laser eyewear tracer. The 3D laser eyewear tracer performs an edge detection function that, as shown by a dark line in FIG. 121, forms an edge profile of the single lens of FIG. 120. This unprocessed edge profile of the single lens of FIG. 120 is shown in greater detail in FIG. 122. The 3D laser eyewear tracer performs a function that removes laser scattering and noise within the roughness of the edge profile to produce, in FIG. 123, a processed edge profile of the single lens of FIG. 120. FIG. 124 shows a comparison of lens traces from a prior art mechanical tracer (left in the figure) and the 3D laser eyewear tracer (right in the figure) by overlaying the two and showing this overlay therebetween. There is little or no visible difference and, in practice, the 3D laser eyewear tracer performs significantly better.

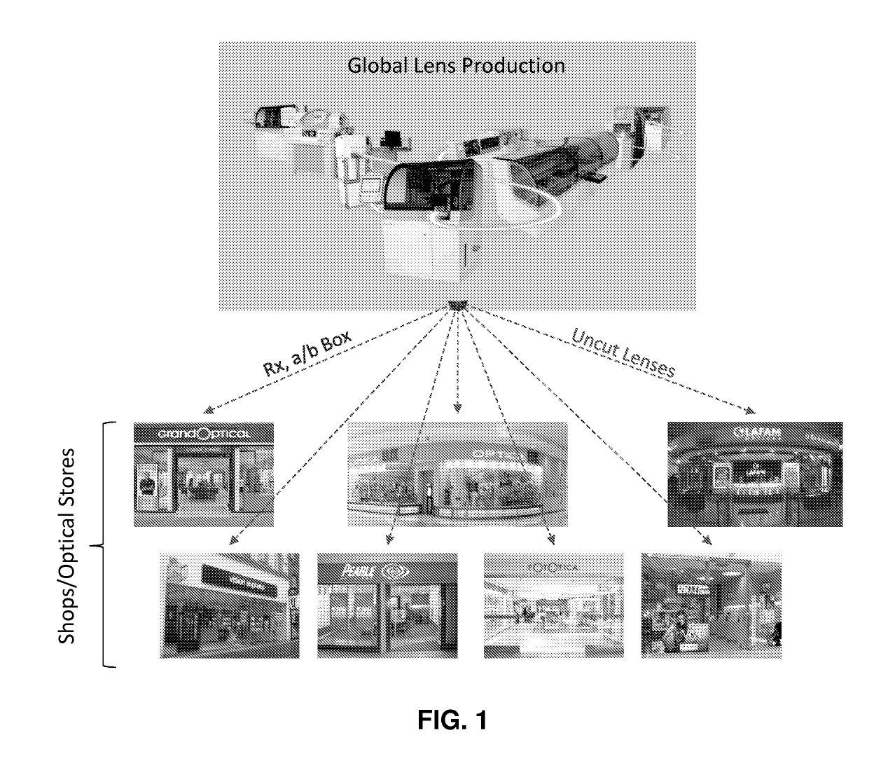

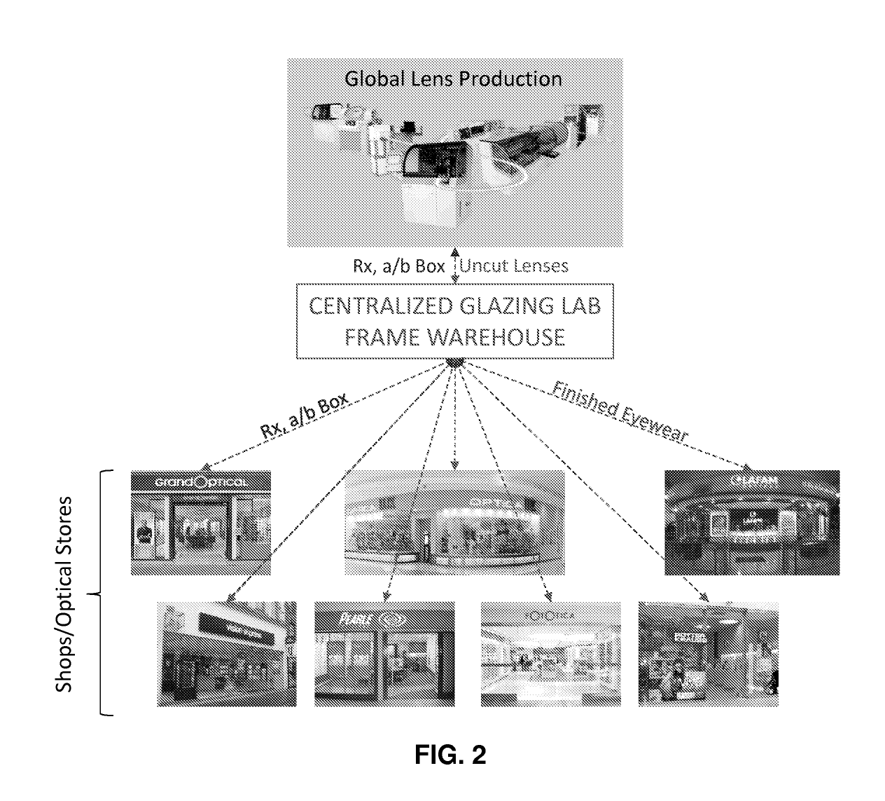

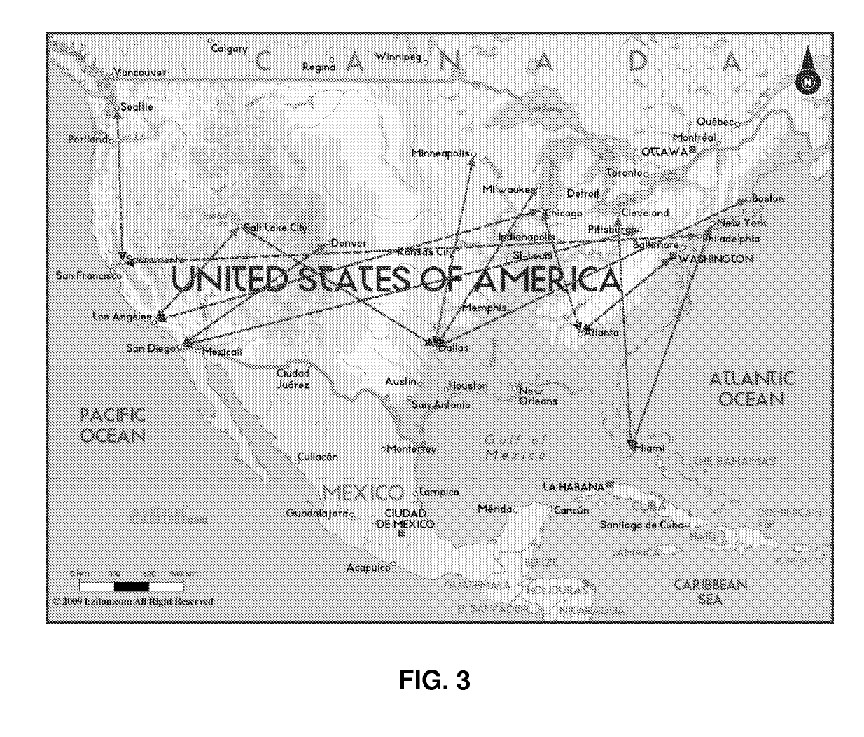

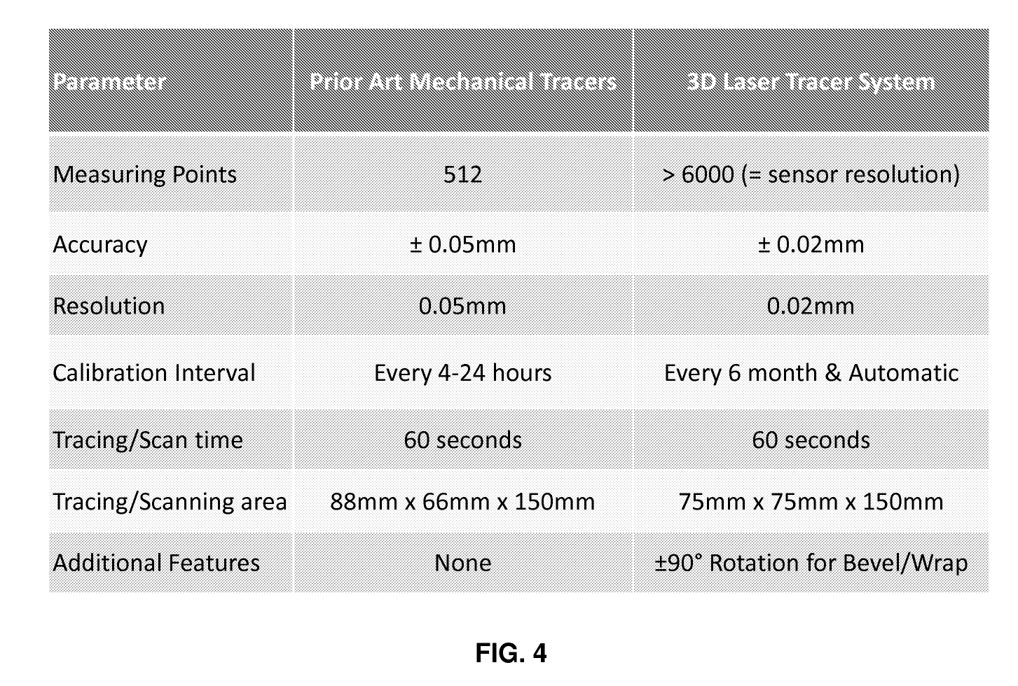

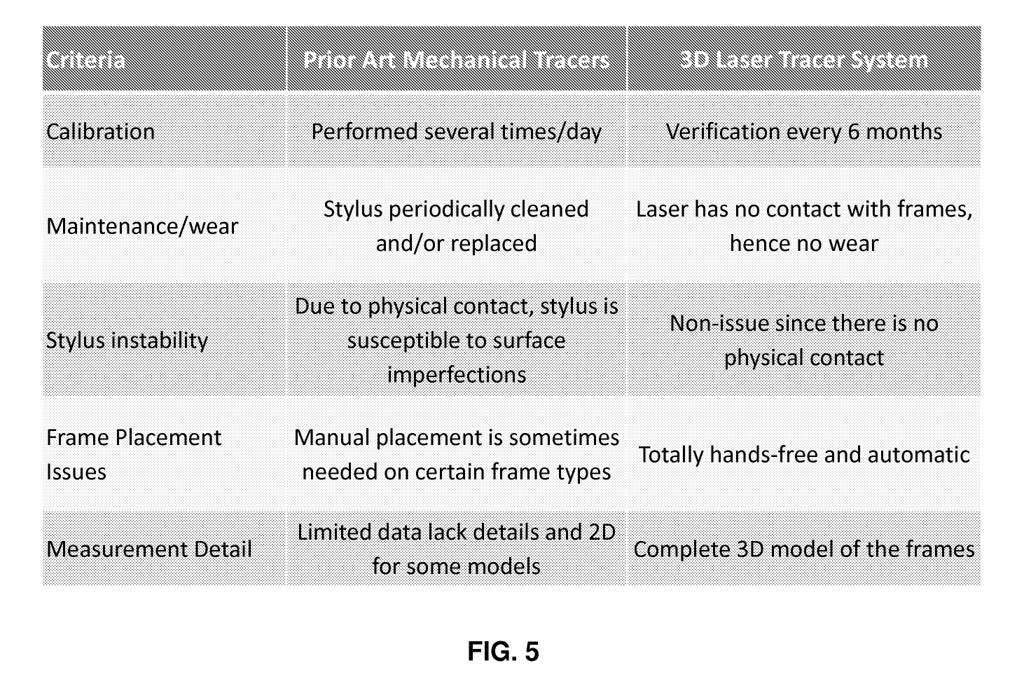

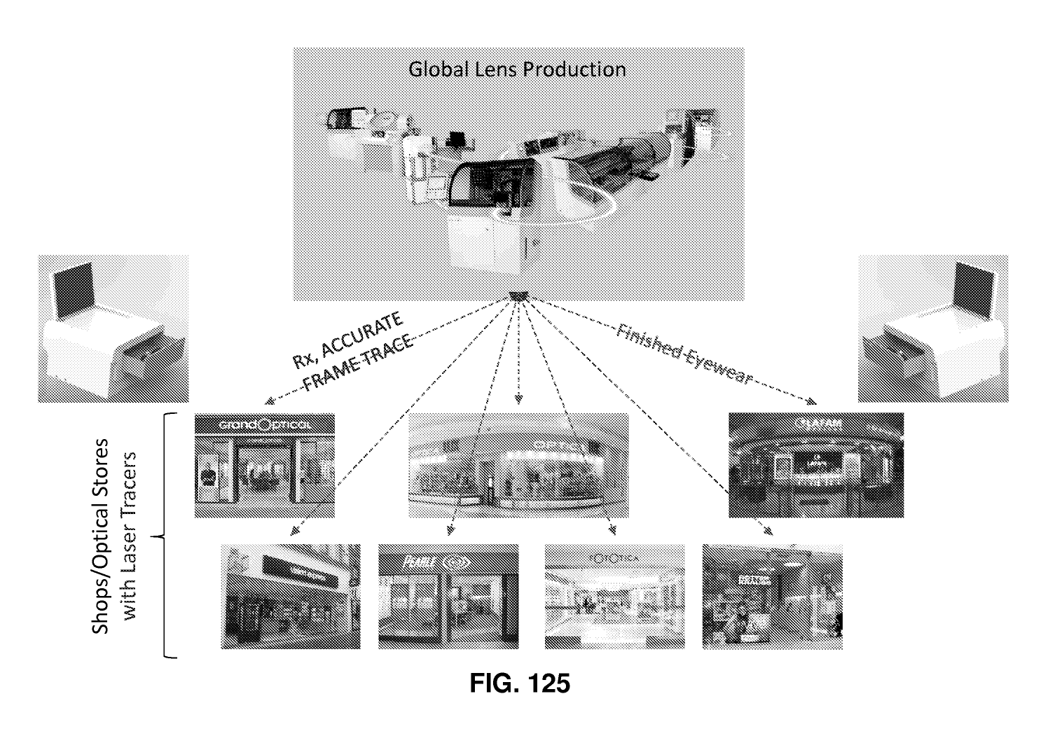

As set forth above with respect to the prior art lens manufacturing processes of FIGS. 1 and 2, frames needed to be shipped and that this multiple shipments were highly undesirable. With the 3D laser eyewear tracer, now, only an electronic copy of the prescription data along with an electronic copy of the output from the 3D laser eyewear tracer is necessary to create finished eyewear. This means that any location having the 3D laser eyewear tracer can send electronically all information that is needed for a lab to return back to the originating location a completed product corresponding to the frame or the lenses shown to the customer. All that is needed when received by the location is for personnel to snap the lenses into the openings within the frame or to attach the lenses to the frame. FIG. 125 diagrammatically shows how the inventive process permits sending only the prescription along with an accurate frame trace to a global lens production lab with the lab sending finished eyewear or edge lenses to be mounted in frame without any shipment at all.

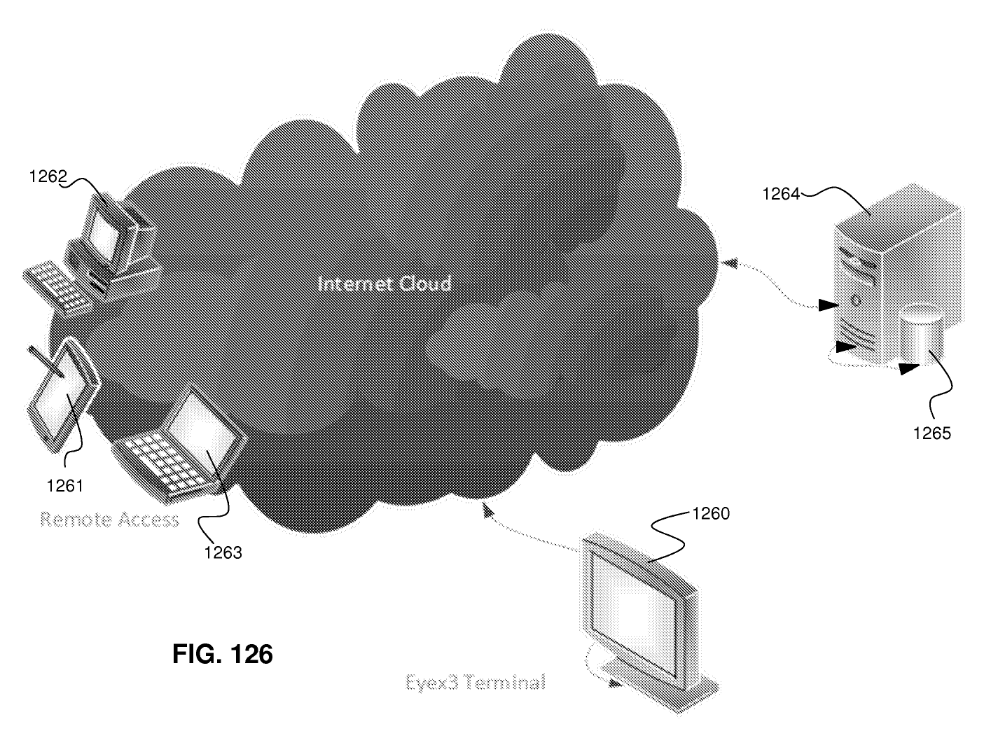

FIGS. 126 to 128 illustrate various ways that the 3D laser eyewear tracer can communicate to other devices and/or locations. FIG. 126 shows a cloud-based communication structure between the 3D laser eyewear tracer 1260 and a cloud file server through an Internet cloud. In this configuration, frame trace data can be remotely accessed by a handheld device 1261, a terminal 1262, or a computer 1263, for example. In particular, the 3D laser eyewear tracer (aka, Eyex3) generates the 3D representation of the scanned eyewear, e.g., in VCA format, and saves it as a data file. This data file then can be stored locally or it can be made accessible anywhere. In the latter case, the 3D laser eyewear tracer transmits the data file to a cloud file server 1264, which stores the data file in a database 1265, diagrammatically depicted in FIG. 126 separate from the cloud file server 1261 for illustrative purposes. The data file can, therefore, be accessed by anyone through the cloud if access permission to the database 1265 is granted. This means that any lab having connectivity to the Internet (wired or wirelessly) can access the data file regardless of where the 3D laser eyewear tracer 1260 is located.

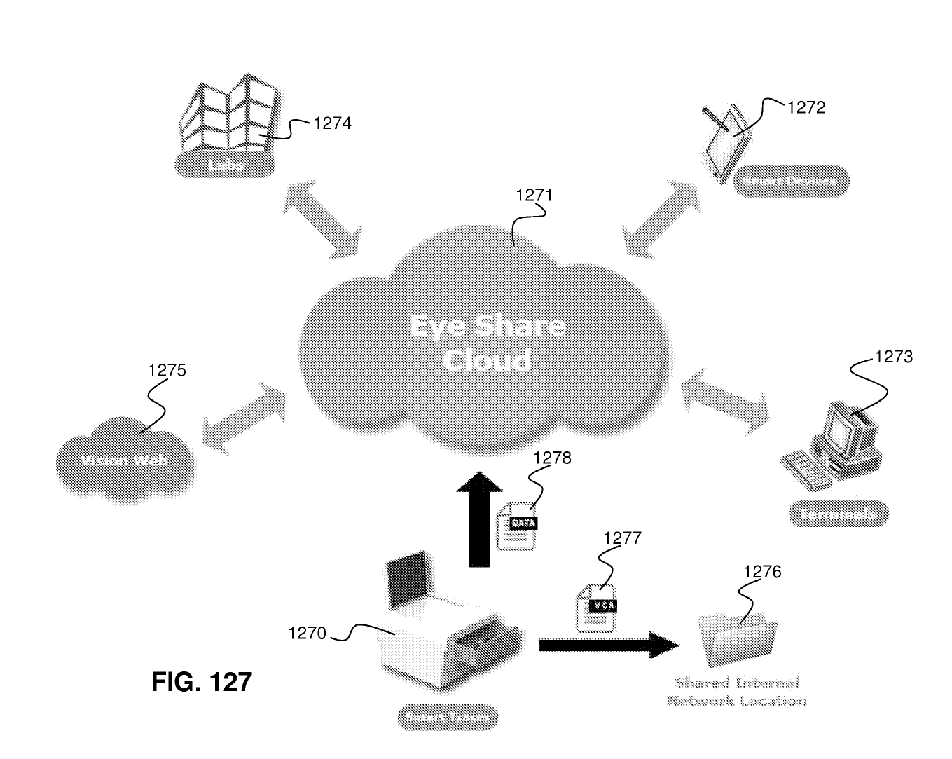

FIG. 127 details cloud-based data sharing between the 3D laser eyewear tracer 1270 and any other device, whether co-located or at a distance. For example, an Eye Share Cloud 1271 on the Internet is able to connect the 3D laser eyewear tracer 1270 to smart devices 1272, to terminals 1273, to optical labs 1274, and/or to any other web resource 1275 that is able to transfer trace data generated by the 3D laser eyewear tracer 1270. In particular, the 3D laser eyewear tracer 1270 generates the 3D representation of the scanned eyewear, e.g., in VCA format, and saves it internally or in a local network 1276 as a data file 1277. This data file 1027 can then be made accessible anywhere. In the latter case, the 3D laser eyewear tracer 1278 transmits the data file 1277 as data 1278 through the Eye Share Cloud 1271. Any of the devices or locations 1272, 1273, 1274, 1275 can store or use the data 1278 in any way. For example, the lab 1274 can use the data and the corresponding eye prescription of the customer to cut the lenses and then ship the lenses to the ordering location, e.g., an eyewear storefront. The data file 1277 can, therefore, be accessed by anyone through the cloud if access permission is granted. This means that any lab having connectivity to the Internet (wired or wirelessly) can access the data file regardless of where the 3D laser eyewear tracer 1270 is located.

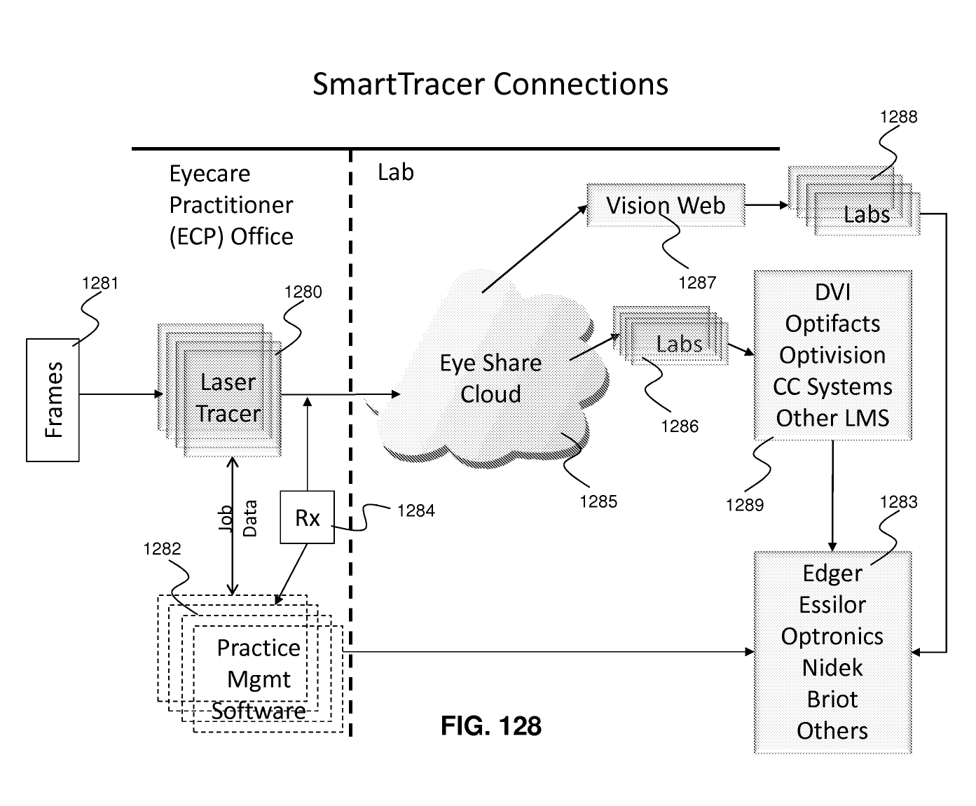

Finally, FIG. 128 illustrates possible connections between the 3D laser eyewear tracer from an eye care practitioner's (ECP) office (to the left of the dashed line in FIG. 128) to shipment of a finished eyewear.

In a first example, the ECP has its own lens edger (such as those made by Essilor, Optronics, Nidek, Briot). The customer selects an eyewear frame 1281 and the ECP scans the eyewear frame 1281 with the 3D laser eyewear tracer 1280. Practice Management Software (PMS) 1282 at the ECP takes the output data from the 3D laser eyewear tracer 1280 along with the customer's prescription 1284 and communicates that data directly to the ECP's lens edger 1283. The lens edger 1283 cuts the lens(es) and the ECP can install the lens(es) in the selected eyewear frame 1281 for delivery to the customer.

In a second example, the ECP sends out the order for the lens(es) to an outside lab. In this scenario, the customer selects the eyewear frame 1281 and the ECP scans the eyewear frame 1281 with the 3D laser eyewear tracer 1280. The 3D laser eyewear tracer 1280 outputs the data file along with the customer's prescription 1284 and communicates that data through the Eye Share Cloud 1285 either directly to a lab 1286 or through another system 1287 that collects orders and sends those manufacturing orders to a lab 1288. In the former case, the lab 1286 sends the order to a Lens Management System 1289 that tracks the manufacturing order and sends it to a lens edger 1283 for shipment of a final product back to the ECP. In the latter case, the lab 1288 manufactures the lens(es) for later edging by a lens edger 1283. Any of the lens edgers 1283 mentioned herein can be co-located or located separately and the multiplicative use of one lens edger 1283 in FIG. 128 is merely for efficient description. This description is, therefore, not intended to indicate that there is only one lens edger 1283. After shipment back to the ECP, the ECP can install the lens(es) in the selected eyewear frame 1281 for delivery to the customer.

Communication by any of the various systems and interfaces of FIGS. 126 to 128 can be wired (e.g., RS232, USB) or wireless (e.g., Bluetooth, cellular) using standard communications protocols.

Any of the systems and methods described herein can be used to create an eyewear database to house all and multiple measurements taken of each eyewear (frame and/or lens), whether for the first time or for the n^{th} time. A neural network can be implemented on the measurement data for each eyewear measured and, as each is scanned again and again, the data for that particular eyewear (e.g., through a SKU) can better predict how the measurement will be for the n^{th}-plus-1 eyewear without having to measure that eyewear again. Further, if the 3D laser eyewear tracer is used to pre-scan all of the manufacturers' eyewear units (e.g., by their SKUs), the systems and methods can be used to collect frame sales data that can be sold to any frame manufacturer for marketing and other financial purposes.

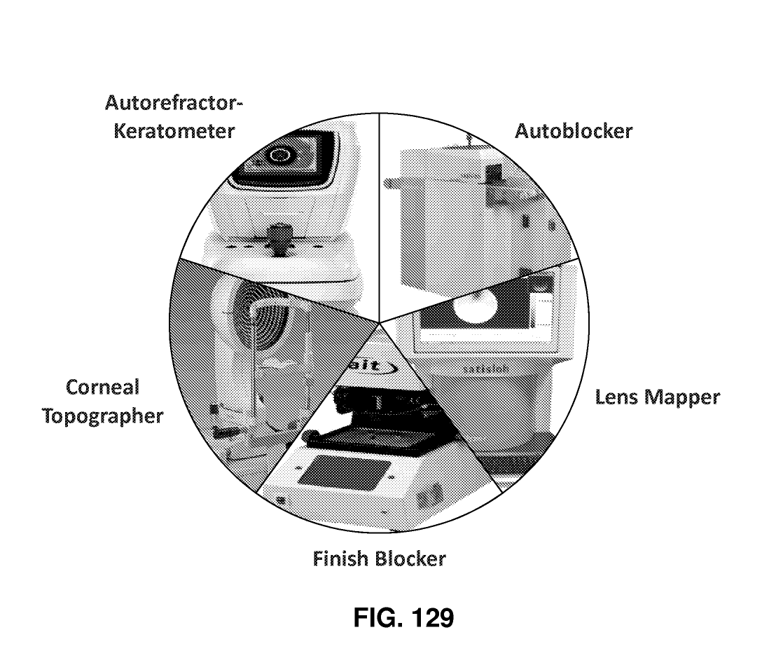

The present disclosure, in FIGS. 129 to 176, illustrates different applications for 3D laser scanning technology. As shown in FIG. 129, multiple applications are provided for ophthalmic instruments using the present platform. An autoblocker is provided for blocking semi-finished lens blanks and providing an accurate front curve measurement. A lens mapper is provided for prescription verification. In one embodiment, a finish blocker can be provided in conjunction with a lens mapper. Multiple applications are also provided for ophthalmic medical devices. The 3D laser scanning platform may provide a corneal topographer. The 3D laser scanning platform may also provide an Autorefractor-Keratometer.

Autoblocker

Manual and automated lens blockers are used in all ophthalmic labs for blocking semi-finished lens blanks. A semi-finished lens blank resembles a hockey puck and is convex on a front portion and concave on a back portion. The process to block a lens includes taking a metal chuck and using an alloy (similar to soldering) to cut down the lens into a thin lens that is then coated, polished, and edged.

Prior art blockers come with prism blocking capabilities, however, these, prior art blockers lack the capability to accurately measure a front curve of the lens. The issues with prior art blockers are due to poor infrared (IR) imaging technology. It is very difficult to use IR to locate the geometric center of the lens accurately, to locate a bifocal segment, and to locate a pad printed ink mark for polarized lenses.

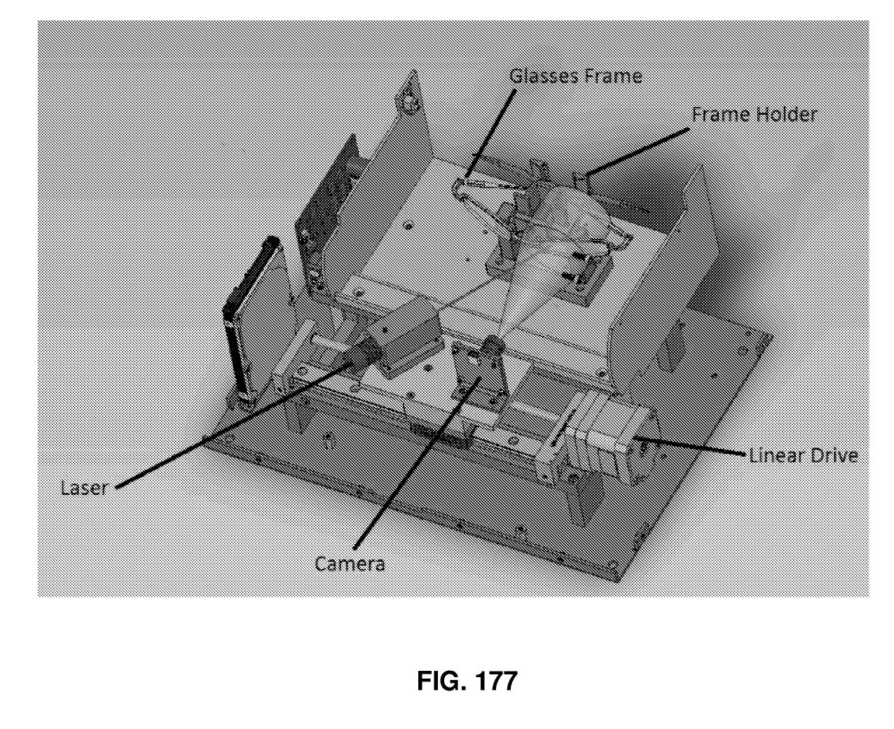

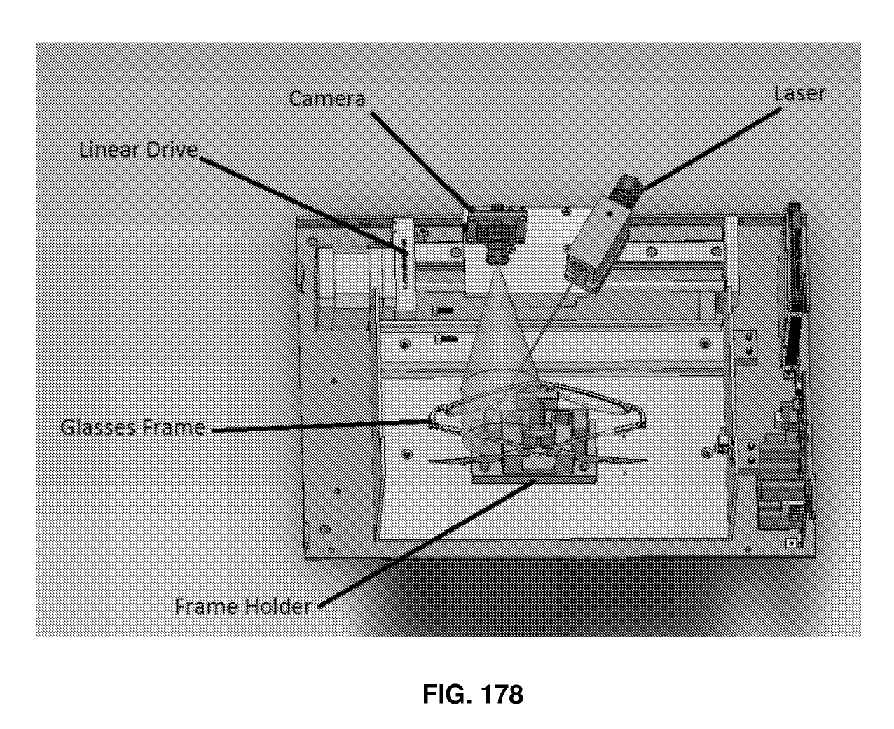

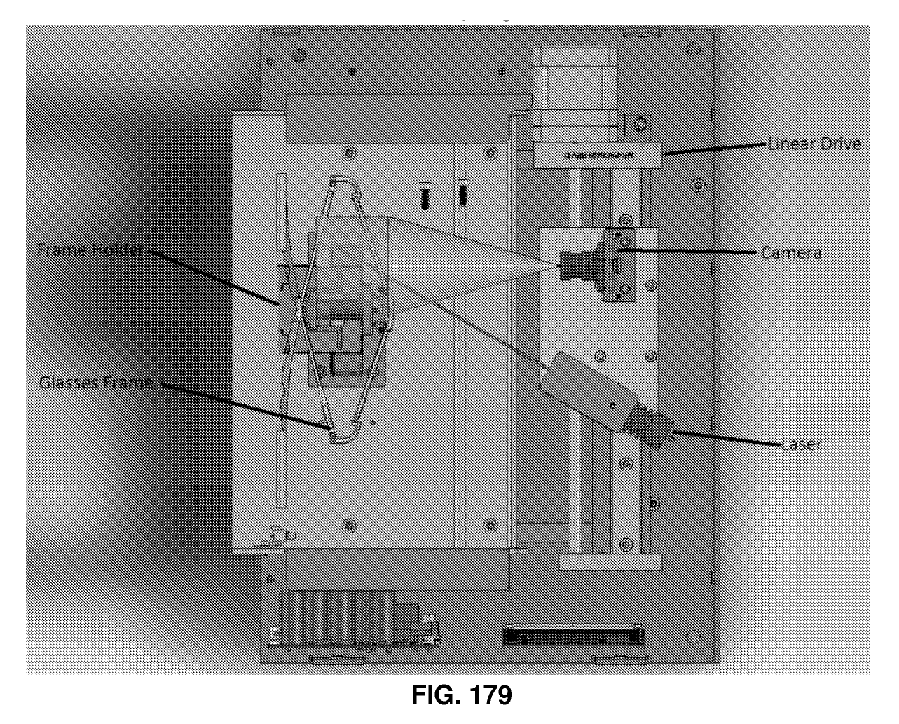

The system shown in FIG. 9 can also be used to provide 3D laser scanning for the Autoblocker application based on triangulation methodology. In this regard, the system of FIG. 9 replaces the prior art device, thereby entirely eliminating the need for an IR array imaging solution. In an exemplary embodiment, the hardware, which includes laser 15, detector 13, and software running on computer 1, uses a laser line. The laser line can be at a green wavelength. The detector 13 is a CMOS camera detector. In this embodiment, the software includes peak detection software, custom edge detection software, custom 3D data analysis software, and VCA DCS digital formatting software. With such components and software, the 3D laser scanner can be configured as shown in FIGS. 32 to 38J where the camera is placed at an angle and the laser is orthogonal to the frame and/or lens. The 3D laser scanner can also be configured as shown in FIGS. 177 to 179, where the camera is orthogonal to the frame and/or lens and the laser is placed at an angle.

The problems inherent in IR light blockers are solved by the present inventive system. The present system includes an accurate and precise blocker that provides digital front curve data and digital coordinates to block semi-finished lens blanks. The laser line scanner, detector, and image processing software is used to provide autoblocking. Triangulation methodology is used to determine a bifocal add segment as it has a different radius and height. Polarized ink marking is detected as the light is absorbed. The front radius of the lens blank is accurately measured. The process to measure the front radius is to scan the lens blank with the laser scanner. The laser line reflected from the front surface is captured by the camera detector and analysis of the 3D data provides the front radius of the lens blank. With accurate front radius measurement, one can easily determine the accurate back radius and cut the back portion for each lens blank. Using 3D laser scanning to provide autoblocker capability allows for lenses that are much more precise and within a tight tolerance that is well below the current ANSI standard.

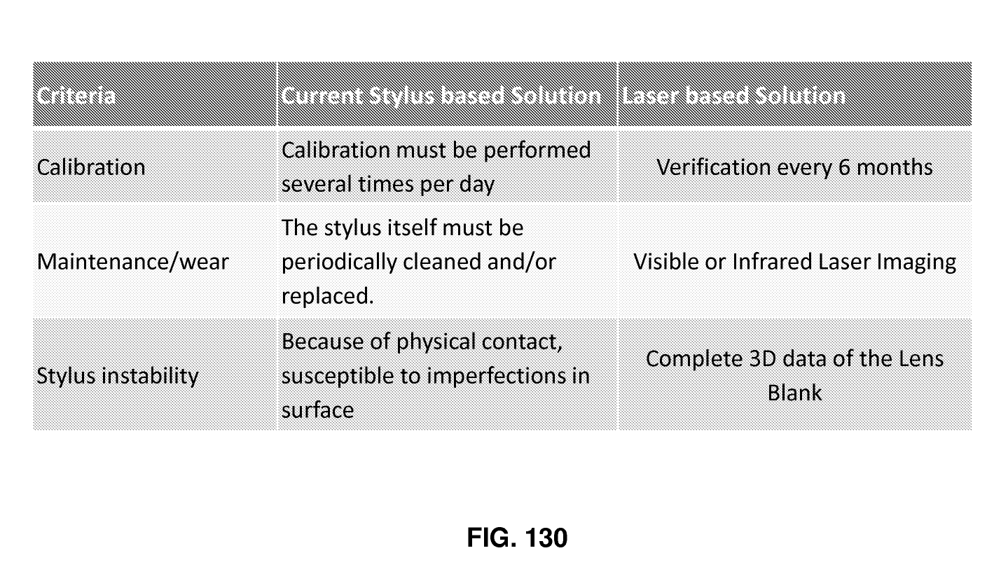

FIG. 130 directly compares calibration, imaging technology, and measurement detail. Each are improved over the prior art. Importantly, calibration is reduced from several time a day to verification every six months. Regarding maintenance, there is no wear because the stylus is removed and, instead, visible and/or infrared laser imaging occurs. There is no issue of stylus instability because there is no longer a stylus.

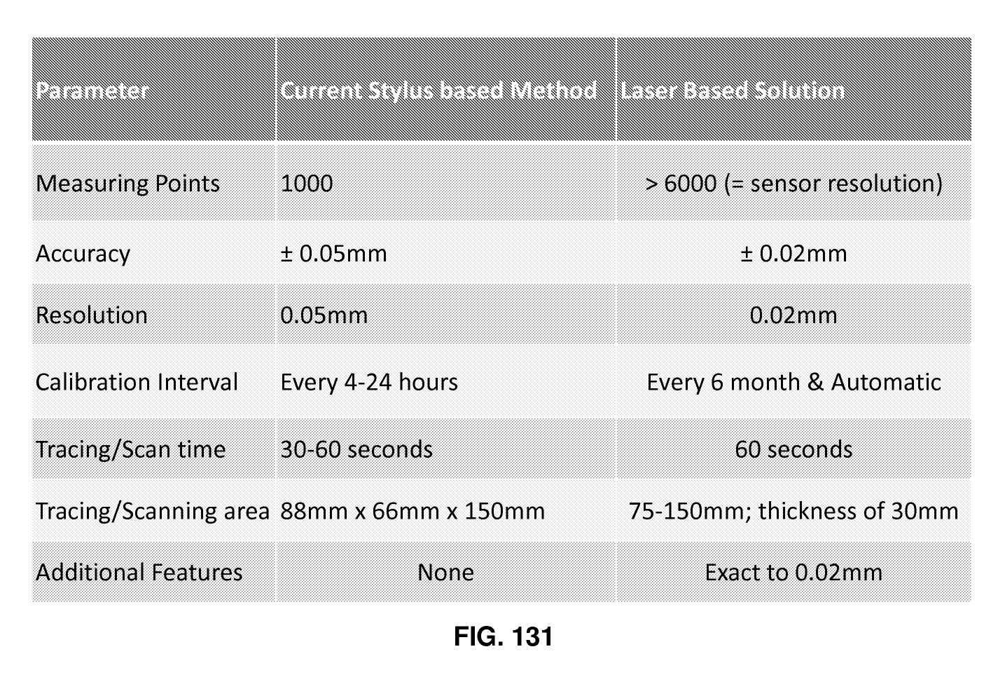

FIG. 131 directly compares the specifications of prior art blockers with an exemplary embodiment of a 3D laser scanning system described herein. The number of measuring points is increased by a factor of at least six. Both accuracy and resolution are two and a half times better. Calibration is vastly improved from a length of hours to twice a year and is turned to automatic calibration. Tracing time is reduced by a factor of six. Simply put, each of the measuring points, accuracy, and resolution are significantly improved over the prior art. Calibration is vastly superior. As for tracing time and scanning area, these parameters are also improved over the prior art.

The blocking method involves 3D laser scanning of the lens surface. Data analysis produces an accurate front surface from the 3D laser scan. The data analysis of the front surface scan is used to recalculate the prescription generation parameters. Data analysis also produces accurate geometric coordinates. Blocking on the optical center or geometric center of the lens is based on the prescription need and utilizes the geometric coordinates. The need for prism blocking is also based on the prescription need and utilizes the geometric coordinates. No change in the cool down time or process is needed. In one exemplary embodiment, prior to blocking, a taping process is performed after the front surface measurement but before the blocking process to the lens to prevent hot spots or other damage to the front surface of the lens.

The advantages of the present autoblocker are numerous. The present autoblocker is more accurate and provides more complete data. Little or no user interaction is needed for scanning or calibration. The present autoblocker provides accurate and precise front curve data to generate the prescription. Accurate and precise digital coordinates are provided for blocking lenses and for de-centering semi-finished (bifocal) lens blanks. Data from the autoblocker can be shared across the cloud, for example, as shown in FIGS. 126, 127, and 128. In addition, the present autoblocker provides significant cost savings over prior art blocking devices.

Lens Mapper

Another instrument that is used in confirming the prescription is a lensometer. Manual and automated lens mappers are used in all ophthalmic labs for prescription verification for single vision and progressive addition lenses (PAL). There are many manual and automated lensometers available in the market. However the manual lensometer is only as good as the quality control expert who knows how to utilize it. Prior art lens mappers rely on manual interpretation of data to verify the prescription. Hence, a large range of tolerance is set up by the ANSI standard. In prior art lens mappers, the prescription range is limited to accurately measure +4 to −4D due to the limitations of the prior art system. For prior art wavefront lens mappers, the resolution is limited to 100s of points. An extended prescription range cannot be accurately measured using prior art lens mapper systems. Additionally, in the case of free-form progressive lenses that possess the add power on the back surface of the lens blank, due to prism thinning that is applied to the design, it is very difficult to accurately measure the add powers in progressive lenses. With the automated lensometer, even those that are based on wavefront analysis, have problems accurately measuring high myopic (minus) or hyperopic (plus) prescriptions.

The system shown in FIG. 9 can also be used to provide 3D laser scanning for the lens mapper application based on triangulation methodology. In this regard, the system of FIG. 9 replaces the prior art device, thereby entirely eliminating the need for a solution using a Hartmann-Shack lenslet array. In an exemplary embodiment, the hardware, which includes laser 15, detector 13, and software running on computer 1, uses a laser line. The laser line can be at a green wavelength. The detector 13 is a CMOS camera detector. In this embodiment, the software includes peak detection software, custom edge detection software, custom 3D data analysis software, and VCA DCS digital formatting software. With such components and software, the 3D laser scanner can be configured as shown in FIGS. 32 to 38J where the camera is placed at an angle and the laser is orthogonal to the frame and/or lens. The 3D laser scanner can also be configured as shown in FIGS. 177 to 179, where the camera is orthogonal to the frame and/or lens and the laser is placed at an angle.

The lens mapper of the present disclosure is accurate and precise and provides digital front curve data, digital back curve data, prescription verification, and a sphero-cylinder power map. The present lens mapper overcomes the problem of prior art lens mappers using laser-scanner triangulation methodology to accurately measure the front and back radius and thickness along the entire dimension of the finished lens. The process begins with scanning the lens with the laser scanner. Most of the laser line reflects from the front surface and the rest of the laser line passes through the transparent lens and reflects back from the back surface. Both the front and back reflected laser lines are captured by the camera detector and further analysis of the front and back 3D data provides the front and back radii of the lens and determining the difference between the front and back radii provides the thickness of the lens. Using optical software such as Zemax, Oslo, Code V, or any other algorithm, one can easily compute the sphere, cylinder, and axis prescription and the add power at any given point on the finished prescription lens.

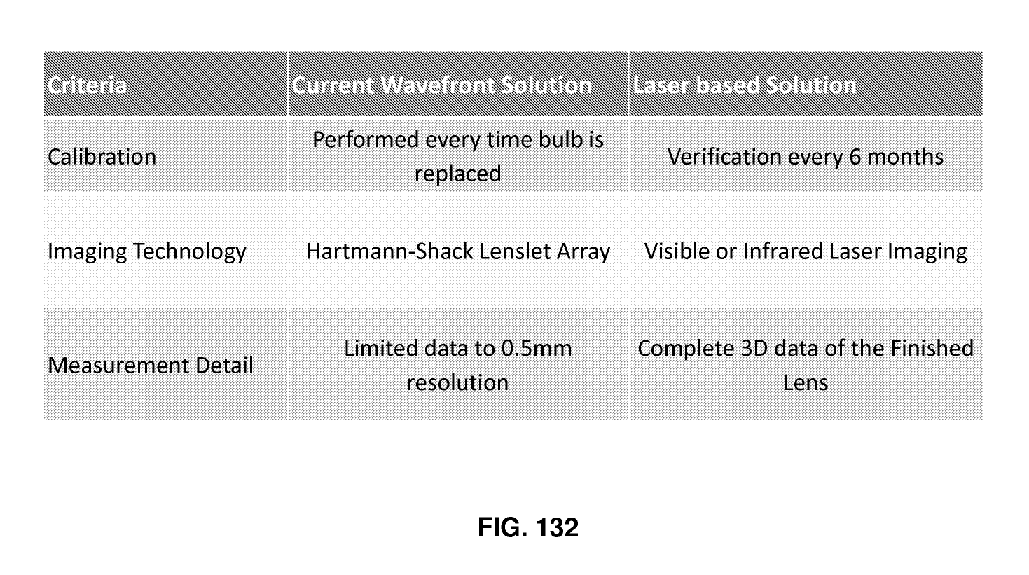

FIG. 132 directly compares calibration, imaging technology, and measurement detail. As before, calibration times change from bulb replacement to simple verification twice a year. The imaging technology is limited to a particular array instead of using laser imaging. Finally, a 3D data of the finished lens provides the image instead of being limited to 0.5 mm resolution. Thus, each characteristic is improved over the prior art.

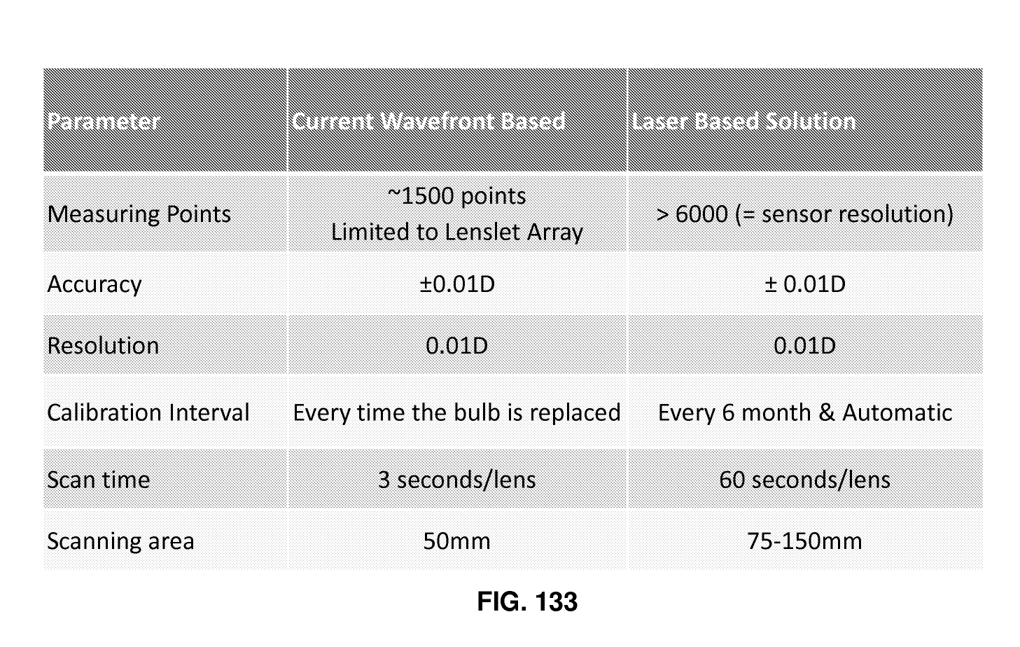

FIG. 133 directly compares the specifications of prior wavefront-based lens mappers with an exemplary embodiment of a 3D laser scanning system described herein. The number of measuring points, accuracy, and resolution are significantly improved over the prior art. First, the number of measuring points is improved by a factor of four. The accuracy and resolution of the laser-based solution are comparable with the wave-front based solution. Calibration is vastly superior and is measured twice a year instead of much more frequent times. As for scanning time and scanning area, the scanning time is longer for the laser-based solution (a few seconds more), however, the scanning area can be up to 3 times as large as the scanning area of the wavefront-based solution.

Advantages of the present lens mapper are numerous. The present lens mapper is more accurate and provides more complete data. Little or no user interaction is needed for scanning or calibration. The present lens mapper provides accurate and precise front curve data and back curve data for prescription verification and for mapping sphero-cylinder power. Integration with Zemax or other optical software package provides an accurate prescription to 0.01D. Data from the lens mapper can be shared across the cloud, for example, as shown in FIGS. 126, 127, and 128. In addition, the present lens mapper provides significant cost savings over prior art lens mappers and is easy to use.

Lens Mapper/Finish Blocker

Another machine that is used to generate prescription eyewear is the finish blocker. Manual and automated finish blockers are used in all ophthalmic labs for blocking single vision and PAL. Prior art manual blockers rely on manually aligning the finished lenses prior to edging. The automated finish blocker relies on imaging technology to align the finished lenses. The imaging technology uses a camera detection system using visible light to capture the optical center that is marked by the operator or utilizes the lensometer to determine the optical center and then the camera aligns the lens based on the output of the lensometer. Prior art finish blockers require manual input to align laser engraving marks or pen marks to block coated finished lenses for the edging process to trim the lens down to the shape of the frame in which the lens is to be mounted. One problem inherent in prior art finish blockers is that manual processes require the eyesight of personnel to be good for performing optical and lensometer work. Further issues with prior art finish blockers include poor training of personnel who are using the blocker. There is also a poor imaging process for analyzing the blocking center.

The system shown in FIG. 9 can also be used to provide 3D laser scanning for the lens mapper and/or finish blocker application based on triangulation methodology. In this regard, the system of FIG. 9 replaces the prior art device, thereby entirely eliminating the need for a solution using a Hartmann-Shack lenslet array. In an exemplary embodiment, the hardware, which includes laser 15, detector 13, and software running on computer 1, uses a laser line. The laser line can be at a green wavelength. The detector 13 is a CMOS camera detector. In this embodiment, the software includes peak detection software, custom edge detection software, custom 3D data analysis software, and VCA DCS digital formatting software. With such components and software, the 3D laser scanner can be configured as shown in FIGS. 32 to 38J where the camera is placed at an angle and the laser is orthogonal to the frame and/or lens. The 3D laser scanner can also be configured as shown in FIGS. 177 to 179, where the camera is orthogonal to the frame and/or lens and the laser is placed at an angle.

The present finish blocker overcomes the issues in prior art finish blockers by using a laser scanning detection process based on triangulation methodology. The present finish blocker is accurate and precise and can be combined with the lens mapper to provide digital prescription verification and digital coordinates of the blocking location. Front surface radius, geometric coordinates, and the location of exact optical center is much more precise using the laser scanning system because the resolution of data is 20 microns or better.

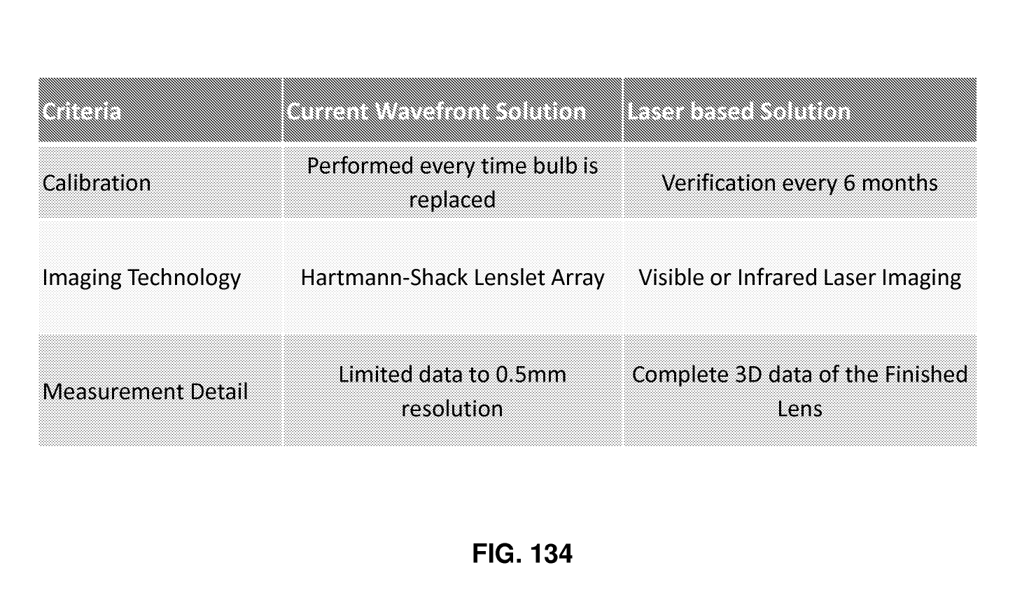

FIG. 134 directly compares calibration, imaging technology, and measurement detail. As before, calibration times change from bulb replacement to simple verification twice a year. The imaging technology is limited to a particular array instead of using laser imaging. Finally, a 3D data of the finished lens provides the image instead of being limited to 0.5 mm resolution. Thus, each characteristic is improved over the prior art.

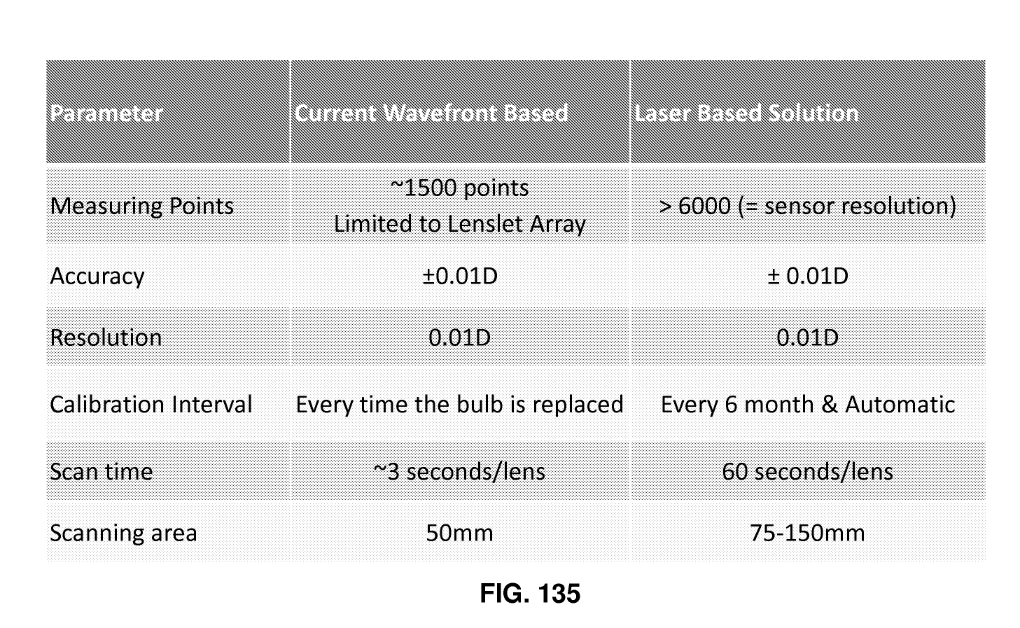

FIG. 135 directly compares the specifications of prior finished blockers with an exemplary embodiment of a 3D laser scanning system described herein. The number of measuring points, accuracy, and resolution are significantly improved over the prior art. First, the number of measuring points is improved by a factor of four. The accuracy and resolution of the laser-based solution are comparable with the wave-front based solution. Calibration is vastly superior and is measured twice a year instead of much more frequent times. As for scanning time and scanning area, the scanning time is longer for the laser-based solution (a few seconds more), however, the scanning area can be up to 3 times as large as the scanning area of the wavefront-based solution.

Advantages of the present lens mapper/finish blocker are numerous. The present lens mapper/finish blocker is more accurate and provides more complete data. Little or no user interaction is needed for scanning or calibration. The present lens mapper/finish blocker provides accurate and precise prescription verification and digital coordinates of the prescription power. Data from the lens mapper/finish blocker can be shared across the cloud, for example, as shown in FIGS. 126, 127, and 128. In addition, the present lens mapper/finish blocker provides significant cost savings over prior art lens mappers and is easy to use.

Corneal Topographer

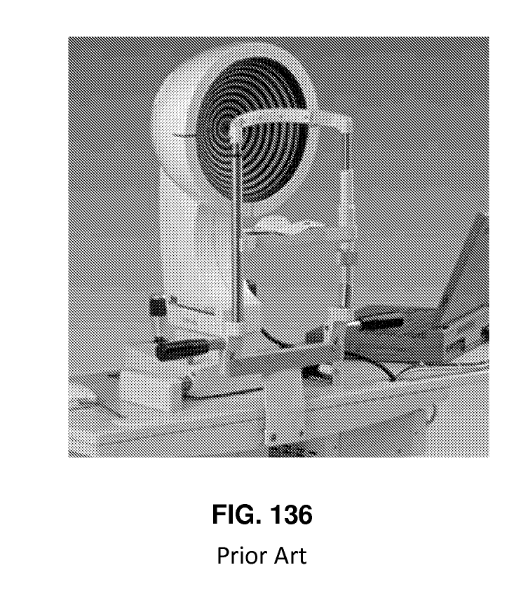

One of the most common medical devices in the optometrist's office is the Corneal Topographer. It is a device that provides the curvature of a patient's eye cornea. Most common Corneal Topographers possess a large placido disk in the shape of a large bowl with an array of IR emitters circularly disposed inside the bowl. The patient is asked to look at the center of the bowl where a screen displays a hot air balloon image that is slightly fogged to place the eye in a relaxed state. The IR light points are reflected from the front surface of the cornea and are detected by an IR sensitive camera to create a topographical map of the cornea. The entire setup takes up too much space in Optometrists' offices and the process is time-consuming. A chin rest is required for alignment of the patient to the prior art device. This alignment is important in determining cylinder axis. The prior art corneal topographer uses a poor imaging process and provides low resolution data and an example of one is shown in FIG. 136.

A simpler solution uses the hereindescribed laser-scanning devices and methods that are based on triangulation methodology. The corneal topographer utilizing the instant laser scanning is accurate and precise and generates a 3D digital surface. The method of providing corneal topography involves using a laser line with camera detection that is placed at an angle to the laser line and this setup captures corneal curvature as the laser line scans from a temporal side to a nasal side or from a top of the eyelid to the bottom of the eye. Such a device with the inventive laser scanning can be made much smaller than prior art corneal topographers and can scan a patient's cornea easily, accurately, and precisely. In one exemplary embodiment, the corneal topographer of the present disclosure can be made into a handheld device.

The system shown in FIG. 9 can also be used to provide 3D laser scanning for the Corneal Topographer application based on triangulation methodology. In this regard, the system of FIG. 9 replaces the prior art device, thereby entirely eliminating the need for a solution using IR LED array imaging. In an exemplary embodiment, the hardware, which includes laser 15, detector 13, and software running on computer 1, uses a laser line. The laser line can be at a green wavelength. The detector 13 is a CMOS camera detector. In this embodiment, the software includes peak detection software, custom edge detection software, custom 3D data analysis software, and VCA DCS digital formatting software. With such components and software, the 3D laser scanner can be configured as shown in FIGS. 32 to 38J where the camera is placed at an angle and the laser is orthogonal to the frame and/or lens. The 3D laser scanner can also be configured as shown in FIGS. 177 to 179, where the camera is orthogonal to the frame and/or lens and the laser is placed at an angle.

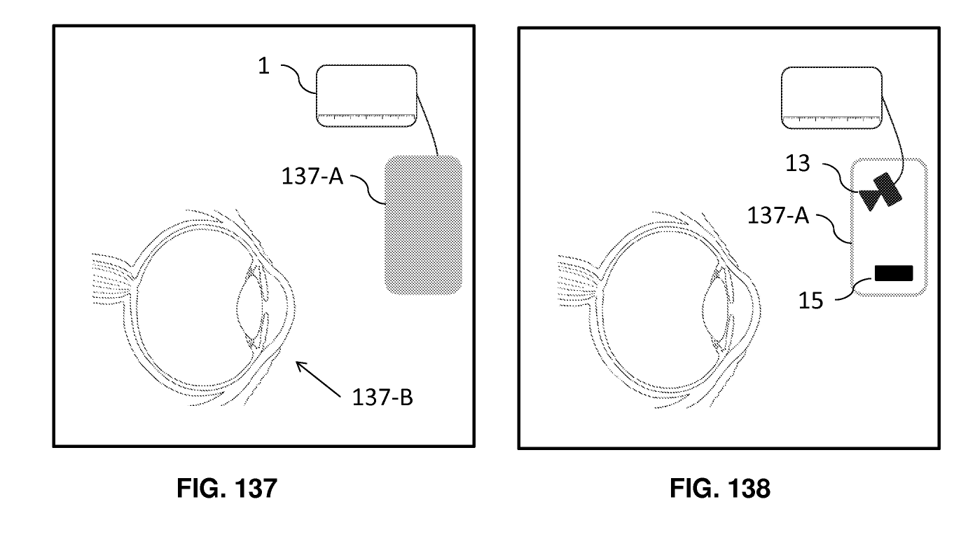

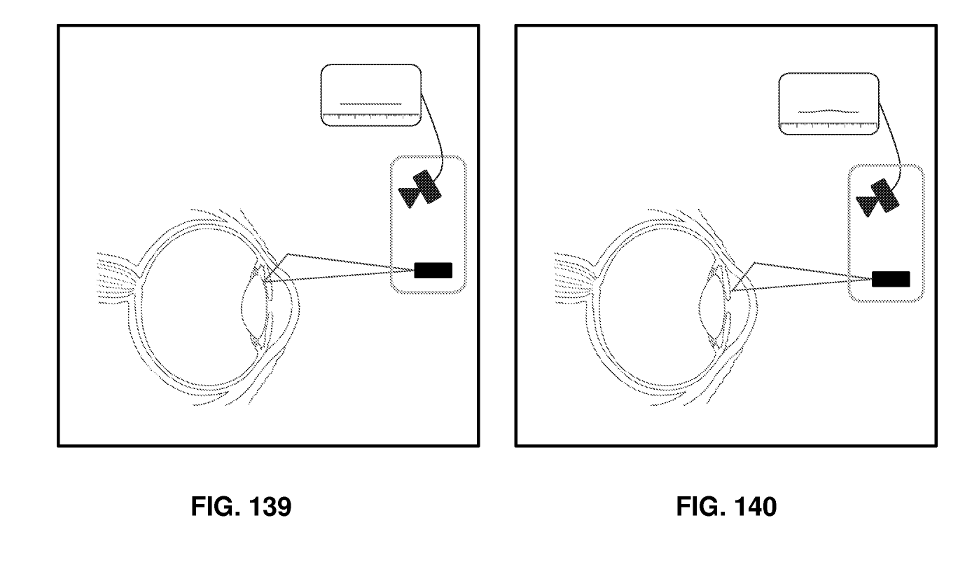

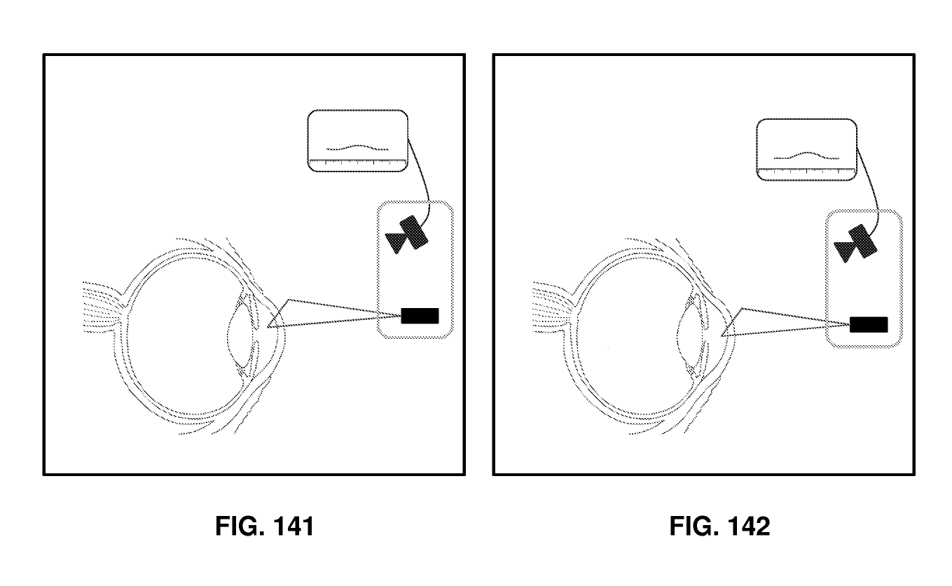

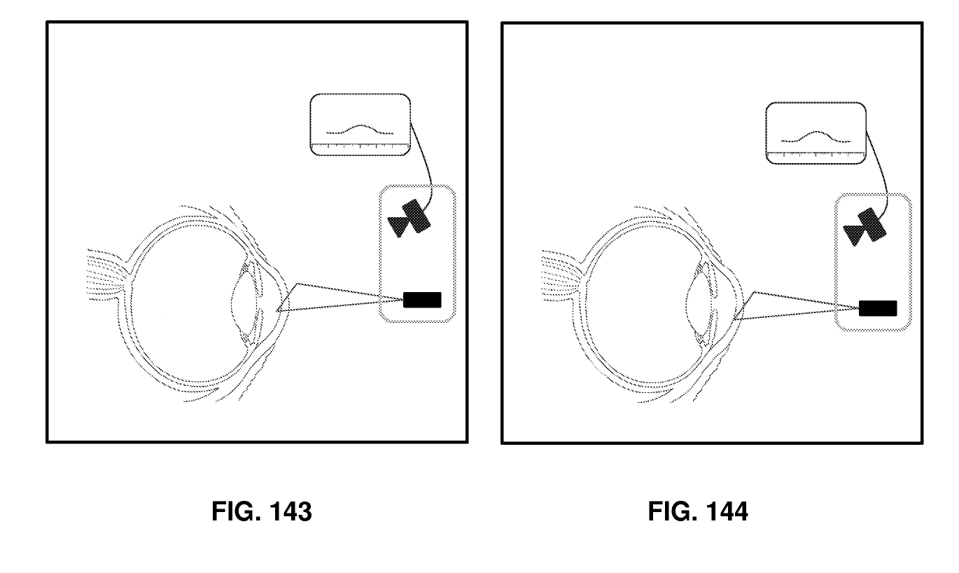

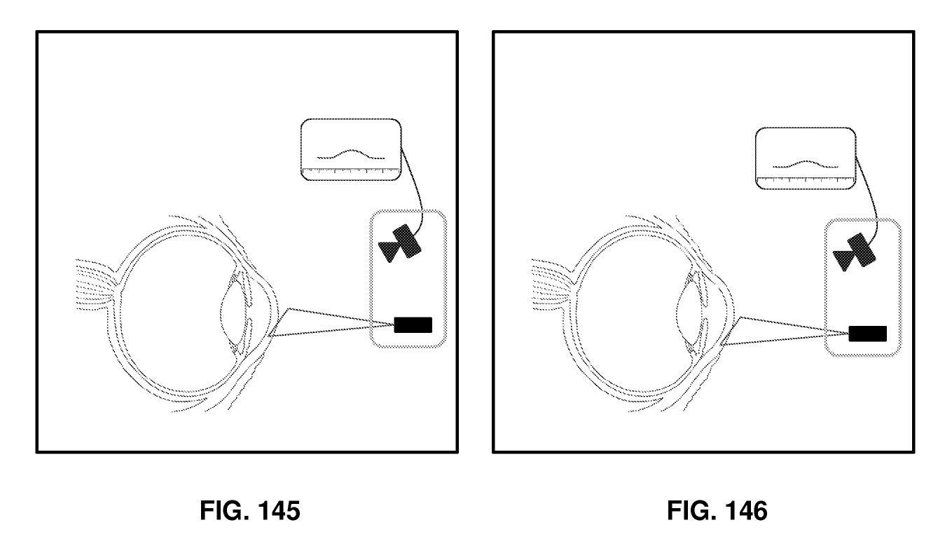

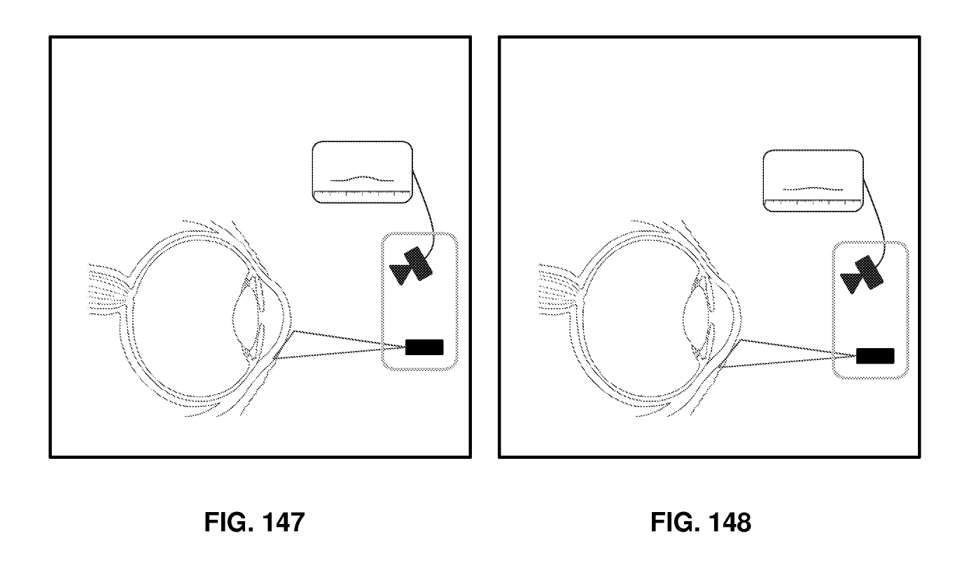

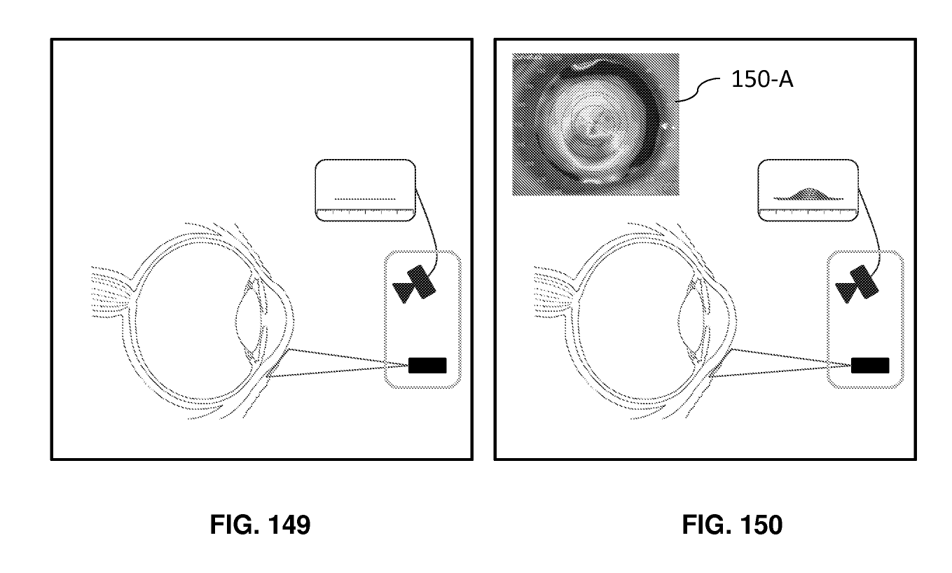

FIGS. 137 to 150 illustrate periodic diagrams of a corneal laser scanning operation with the 3D laser scanner described herein. FIG. 137 shows a laser scanner 137-A, a computer 1, and an eye 137-B. As shown in FIG. 138, laser scanner 137-A includes a detector 13 and a laser 15. In FIG. 139, in this embodiment, the scanning operation begins at a first position at the top of the eyelid. FIG. 140 shows the scanning operation at a second position away from the top of the eyelid in a direction toward the bottom of the eye. FIG. 141 shows the scanning operation at a third position away from the second position in a direction toward the bottom of the eye. FIG. 142 shows the scanning operation at a fourth position away from the third position in a direction toward the bottom of the eye. FIG. 143 shows the scanning operation at a fifth position away from the fourth position in a direction toward the bottom of the eye. FIG. 144 shows the scanning operation at a sixth position away from the fifth position in a direction toward the bottom of the eye. FIG. 145 shows the scanning operation at a seventh position away from the sixth position in a direction toward the bottom of the eye. FIG. 146 shows the scanning operation at an eighth position away from the seventh position in a direction toward the bottom of the eye. FIG. 147 shows the scanning operation at a ninth position away from the eighth position in a direction toward the bottom of the eye. FIG. 148 shows the scanning operation at a tenth position away from the ninth position in a direction toward the bottom of the eye. FIG. 149 shows the scanning operation at an eleventh position away from the tenth position at the bottom of the eye. FIG. 150 shows a topographical map 150-A as captured by computer 1, detector 13, and laser 15 during the scanning operation shown in FIGS. 137 to 149.

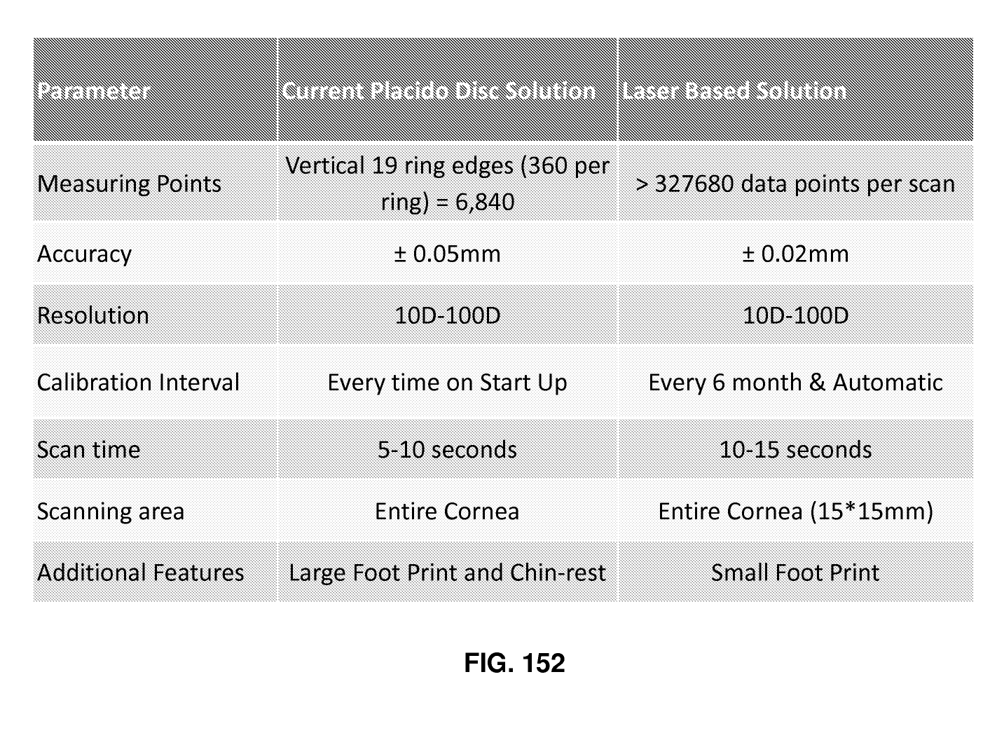

Although the present disclosure describes eleven positions, the number of data points should not be so limited. As shown in FIG. 152, the sensor resolution of the present laser scanner provides 327680 data points per scan.

Further, the scanning operation described in FIGS. 137 to 150 is not limited to scanning from a top eyelid to the bottom of the eye. The scanning operation can be applied by scanning from the bottom of the eye to the top of the eyelid. The scanning operation can also be applied from a temporal side of the eye to a nasal side of the eye. Likewise the scanning operation can also be applied from the nasal side of the eye to the temporal side of the eye.

The present corneal topographer uses a smaller footprint with an IR laser scanner and an IR detector. From the corneal scan, corneal curvature and central K value is measured. In one exemplary embodiment, a chin rest can be used to insure alignment. As stated previously, the alignment is important in determining cylinder power and axis. Cylinder prescription measurement is determined in refractive power along a certain axis. If the two eyes of the patient are not aligned horizontally while utilizing the laser scanning technology, the cylinder power and axis will be incorrect. Data interpolation creates a high resolution corneal topographical map.

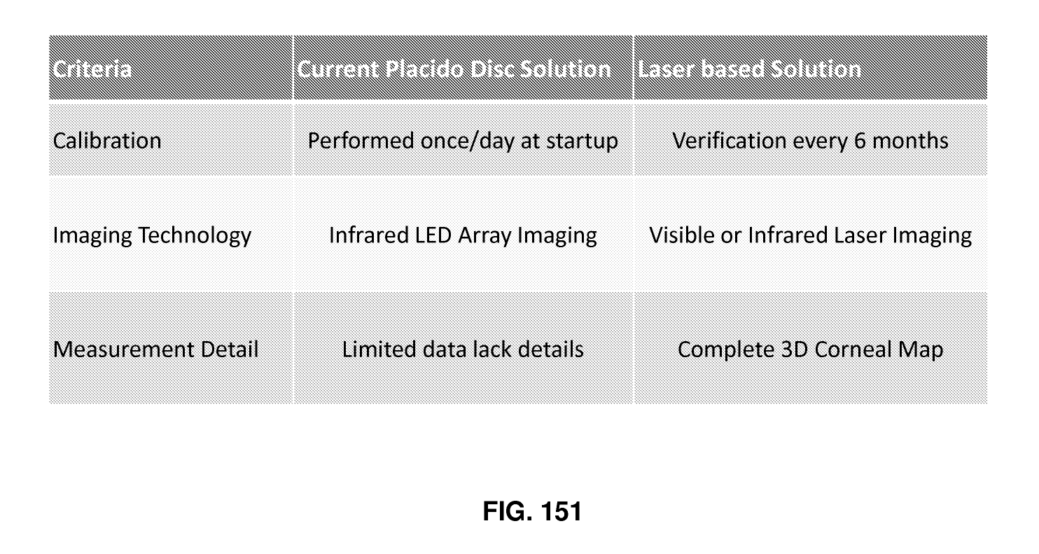

FIG. 151 directly compares calibration, imaging technology, and measurement detail. As before, calibration times change from once each day to simple verification twice a year. The imaging technology is limited to a particular array instead of using laser imaging. Finally, 3D data of the patient's actual cornea provides the image instead of being limited present art low-resolution. Thus, each characteristic is improved over the prior art.

FIG. 152 directly compares the specifications of prior art Placido disc solutions with an exemplary embodiment of a 3D laser scanning system described herein. The number of measuring points and resolution of the Placido disc solution is comparable with the present solution. However, the accuracy is significantly improved over the prior art by a factor of 2.5. Calibration is vastly superior, changing from daily to bi-annualy. As for scanning time and scanning area, these parameters are similar for both solutions. The laser-based solution of the present disclosure, however, requires a much smaller footprint.

Advantages of the present corneal topographer are numerous. The present corneal topographer is more accurate and provides more complete data. Little or no user interaction is needed for scanning or calibration. The present corneal topographer provides accurate and precise prescription verification and digital coordinates of the prescription power. Data from the corneal topographer can be shared across the cloud, for example, as shown in FIGS. 126, 127, and 128. In addition, the present corneal topographer provides significant cost savings over prior art corneal topographers and is easy to use.

Autorefractor-Keratometer

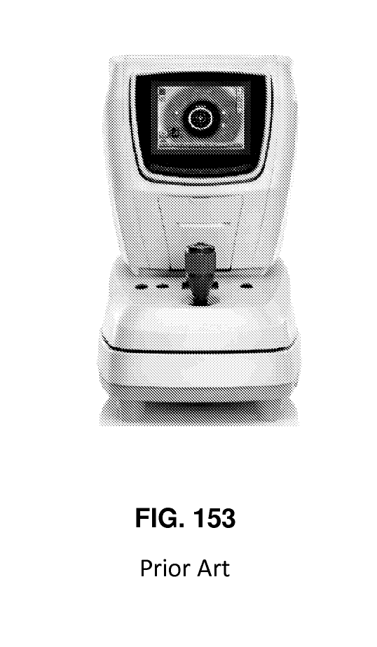

Another common medical device in an eye doctor's office is an Autorefractor with Keratometer. This machine provides an ocular prescription along with a curvature of the patient's cornea. Prior art technology for the Autorefractor is based on a Badal Optometer to determine the objective refraction of the eye. As can be see by the prior art Autorefractor shown in FIG. 153, a large footprint is required, taking up valuable desktop space in the doctor's office. In addition, the prior art device requires use of a chin rest to align the patient to the device. The prior art Autorefractor uses a poor imaging process and provides low resolution data.

The system shown in FIG. 9 can also be used to provide 3D laser scanning for the Autorefractor-Keratometer application based on triangulation methodology. In this regard, the system of FIG. 9 replaces the prior art device, thereby entirely eliminating the need for a separate IR LED Array Imaging. In an exemplary embodiment, the hardware, which includes laser 15, detector 13, and software running on computer 1, uses a laser line. The laser line can be at a green wavelength. The detector 13 is a CMOS camera detector. In this embodiment, the software includes peak detection software, custom edge detection software, custom 3D data analysis software, and VCA DCS digital formatting software. With such components and software, the 3D laser scanner can be configured as shown in FIGS. 32 to 38J where the camera is placed at an angle and the laser is orthogonal to the frame and/or lens. The 3D laser scanner can also be configured as shown in FIGS. 177 to 179, where the camera is orthogonal to the frame and/or lens and the laser is placed at an angle.

The Autorefractor-Keratometer function of the present systems and methods provides a 3D digital surface, determines an ocular axial length along the optical path using a laser rangefinder, and determines pupil diameter. From the pupil diameter measurement, sphero-cylinder power (e.g., sphero-cylinder refraction) for day- and night-time environments can be determined. The corneal topographer described above can replace the Keratometer portion of the Autorefractor in the same manner. With such a configuration, in an exemplary embodiment, the Autorefactor with Keratometer using the inventive laser scanning can be configured in a handheld device. In another exemplary embodiment, an Autorefactor with Keratometer and a corneal topographer can be combined into a single handheld device.

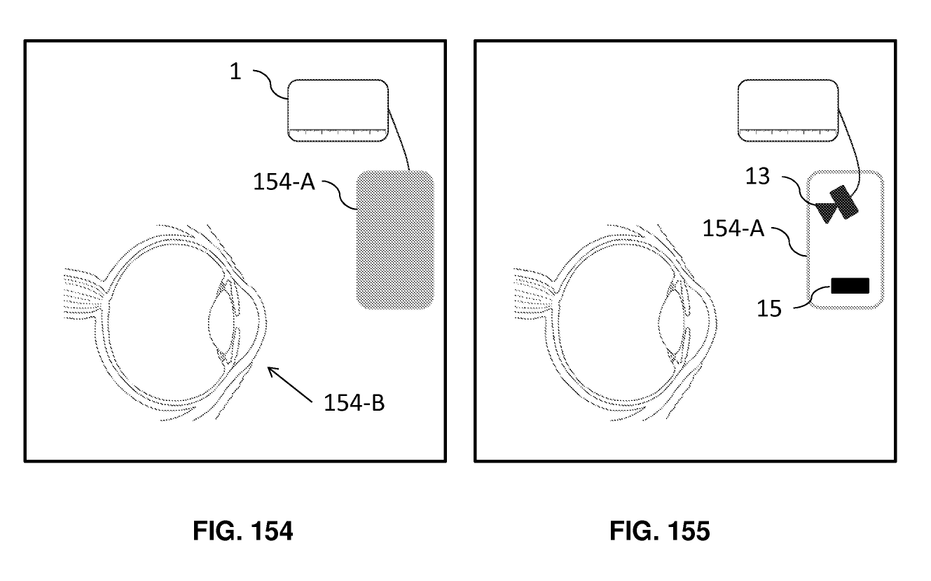

Using a combined Autorefractor-Keratometer and corneal topographer, the corneal topography is measured first as described above in the Corneal Topographer section. FIGS. 154 to 167 correspond to a corneal topography measurement as shown in FIGS. 137 to 150.

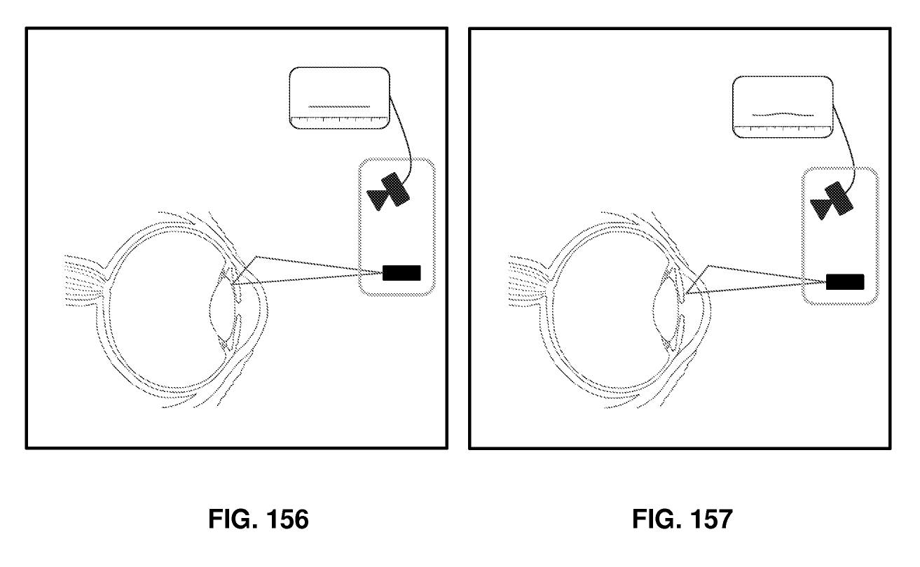

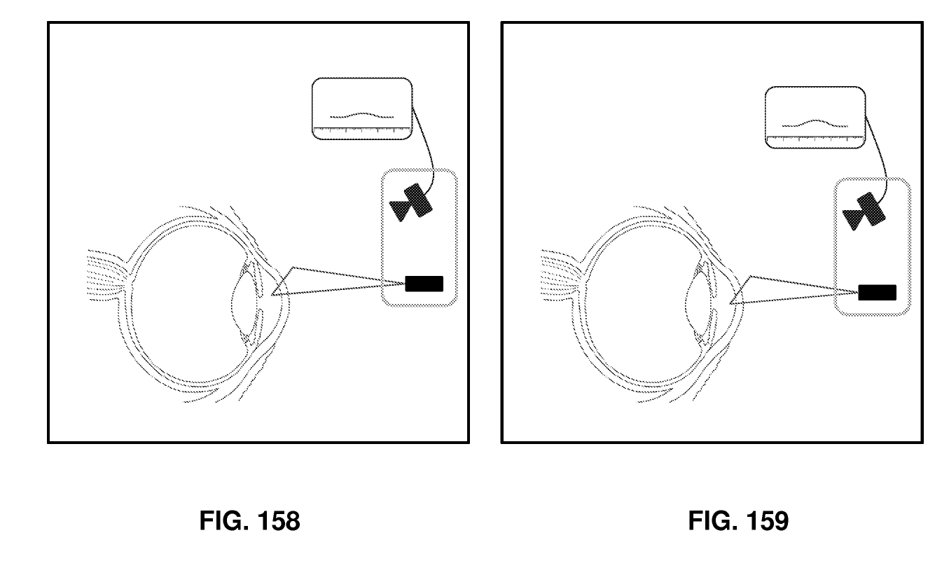

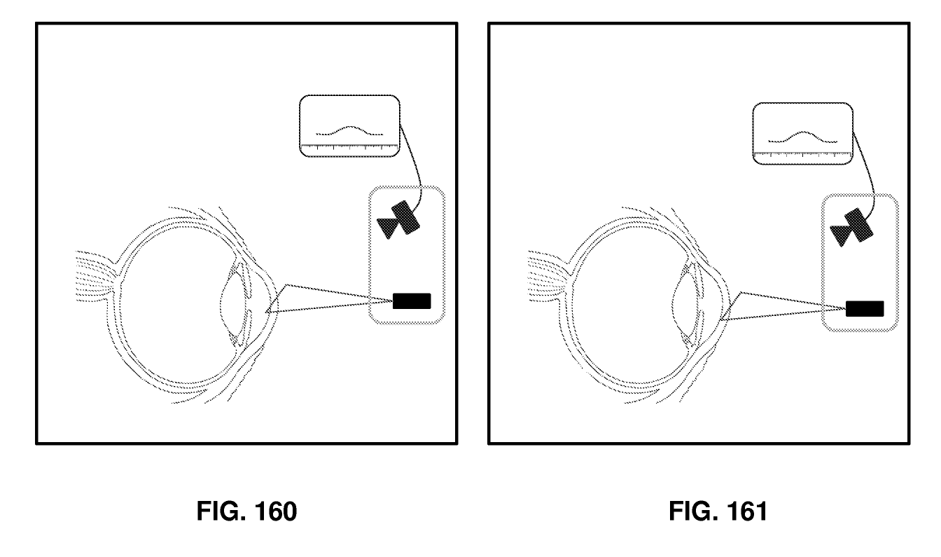

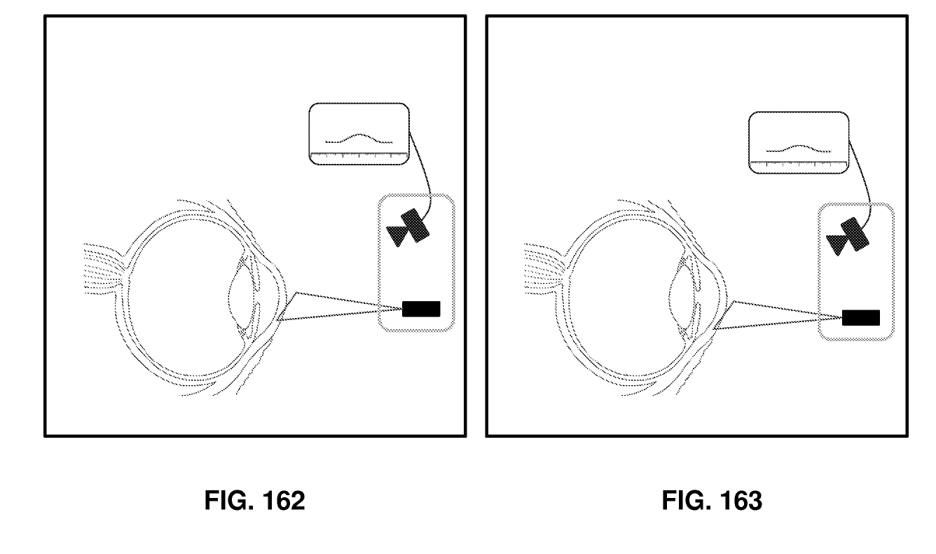

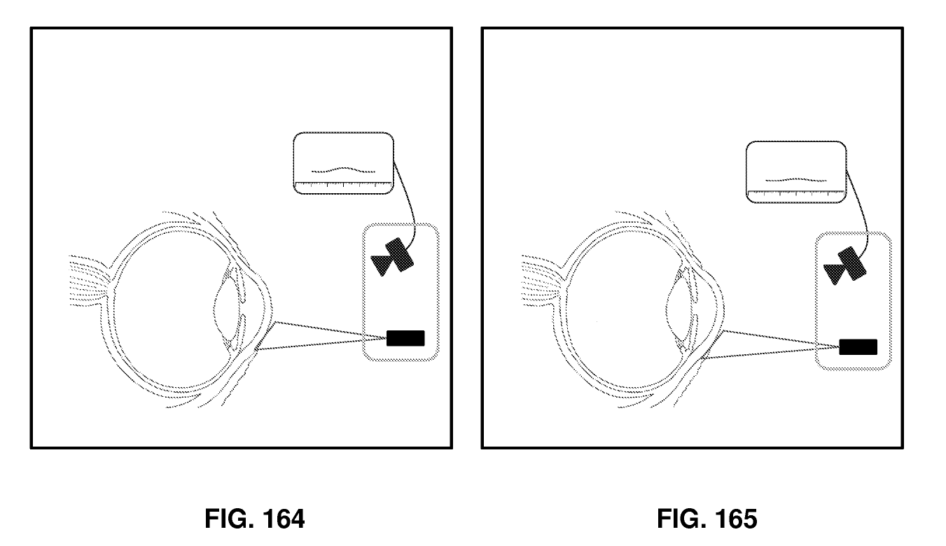

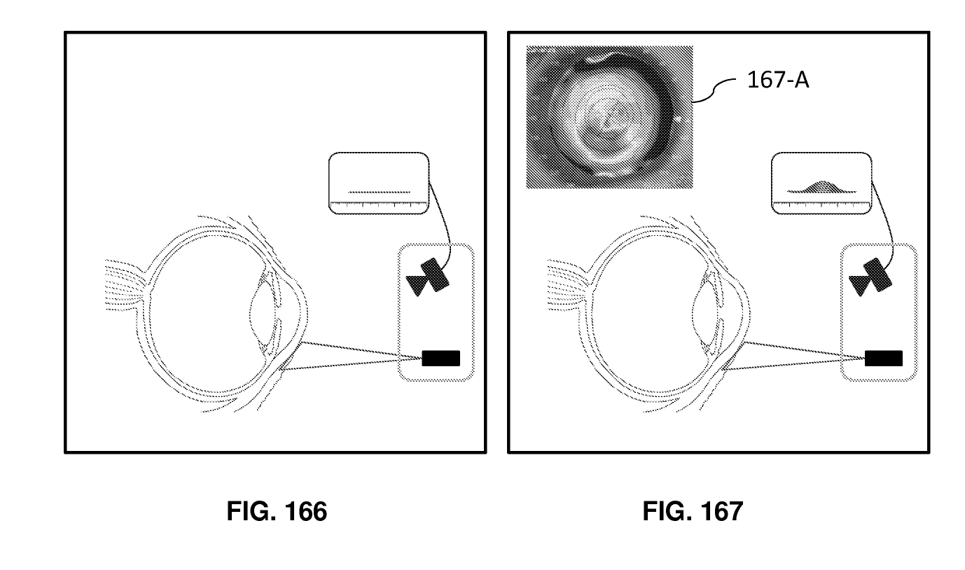

FIGS. 154 to 167 illustrate periodic diagrams of a corneal laser scanning operation with the 3D laser scanner described herein. FIG. 154 shows a laser scanner 154-A, a computer 1, and an eye 154-B. As shown in FIG. 155, laser scanner 154-A includes a detector 13 and a laser 15. In FIG. 156, in this embodiment, the scanning operation begins at a first position at the top of the eyelid. FIG. 157 shows the scanning operation at a second position away from the top of the eyelid in a direction toward the bottom of the eye. FIG. 158 shows the scanning operation at a third position away from the second position in a direction toward the bottom of the eye. FIG. 159 shows the scanning operation at a fourth position away from the third position in a direction toward the bottom of the eye. FIG. 160 shows the scanning operation at a fifth position away from the fourth position in a direction toward the bottom of the eye. FIG. 161 shows the scanning operation at a sixth position away from the fifth position in a direction toward the bottom of the eye. FIG. 162 shows the scanning operation at a seventh position away from the sixth position in a direction toward the bottom of the eye. FIG. 163 shows the scanning operation at an eighth position away from the seventh position in a direction toward the bottom of the eye. FIG. 164 shows the scanning operation at a ninth position away from the eighth position in a direction toward the bottom of the eye. FIG. 165 shows the scanning operation at a tenth position away from the ninth position in a direction toward the bottom of the eye. FIG. 166 shows the scanning operation at an eleventh position away from the tenth position at the bottom of the eye. FIG. 167 shows a topographical map 167-A as captured by computer 1, detector 13, and laser 15 during the scanning operation shown in FIGS. 154 to 166.

Although the present disclosure describes eleven positions, the number of data points should not be so limited. As shown in FIG. 152, the sensor resolution of the present laser scanner provides more than six thousand measuring points.

The Autorefractor-Keratometer with Corneal Topographer uses a smaller footprint when using an IR laser scanner and IR detector according to the embodiments described herein. From the corneal scan, corneal curvature and central K value are measured. In an exemplary embodiment, a chin rest can be used but it is not necessary. Data interpolation on the laser created scan creates a high resolution corneal topographical map. Then, refraction is determined using the IR laser scanner and IR detector for 3D laser scanning.

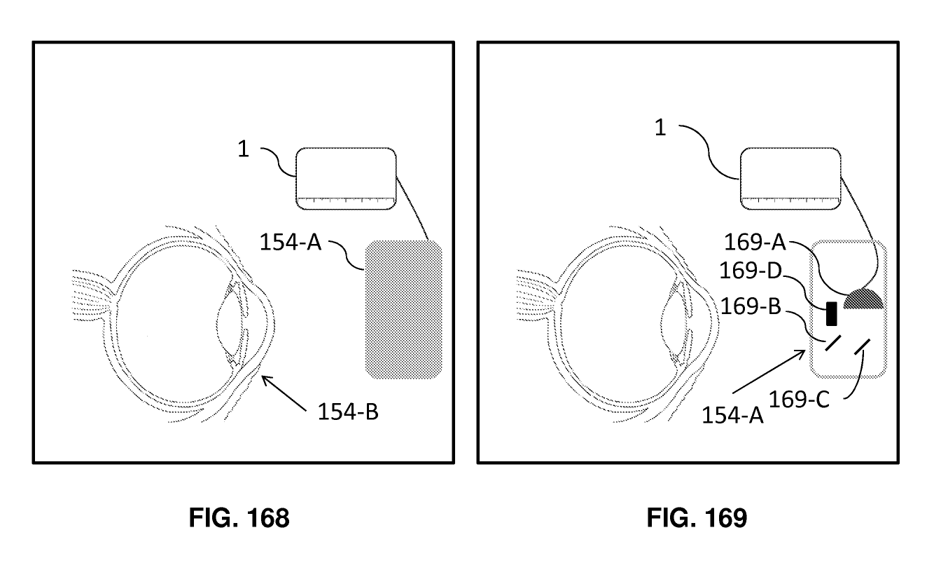

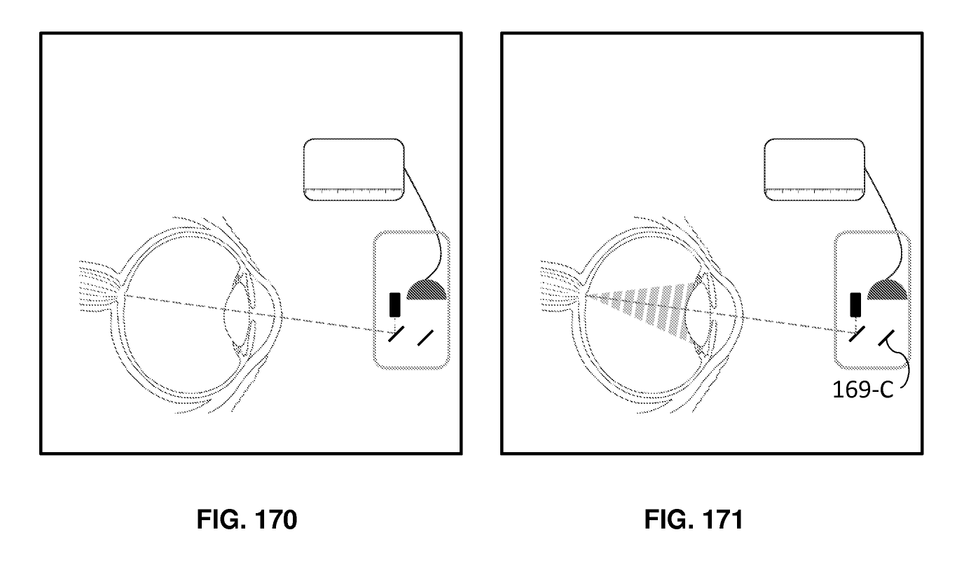

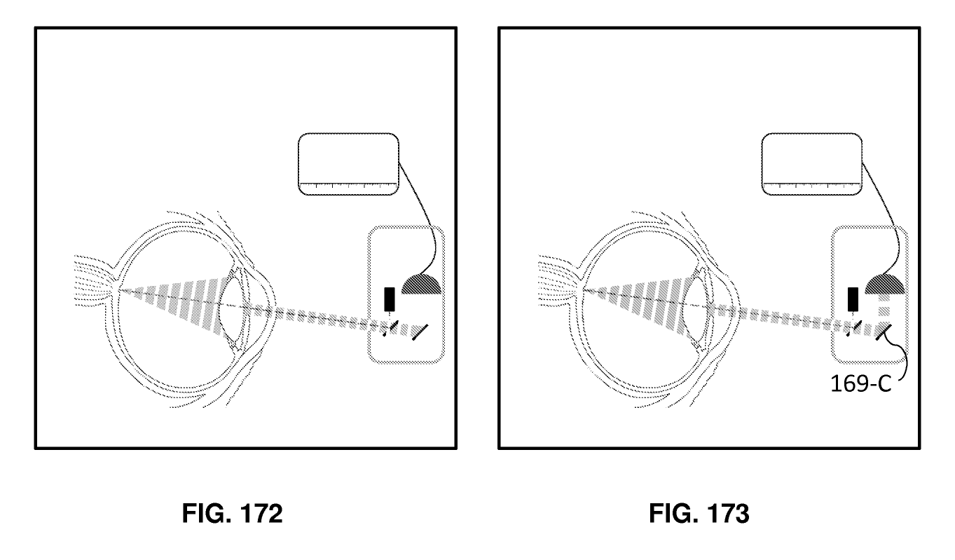

A refraction measurement and pupil diameter measurement is shown in FIGS. 168 to 174. FIG. 168 shows a laser scanner 154-A, a computer 1, and an eye 154-B. In FIG. 169, in addition to detector 13 (not shown) and laser 15 (not shown), the laser scanner 154-A includes detector 169-A, reflectors 169-B and 169-C, and laser 169-D. In this embodiment, the laser 169-D is different from laser 15. In one embodiment, laser 169-D is an IR laser diode. In one embodiment, the detector 169-A is also different from detector 13. In this embodiment, detector 169-A is a detector that works on the Time of Flight (TOF) principle. Refraction is based on a laser rangefinder applying time of flight principles. As shown in FIG. 170, a narrow beam of laser pulse is impinged on the retina (while the patient is looking at a fogged target to relax accommodation) using laser device 169-D and reflector 169-B. FIG. 171 shows the laser pulse being reflected back from the retina to the iris. FIG. 172 shows a portion of the reflected laser pulse passing through the iris and the pupil and on to reflector 169-C. The portion of the reflected laser pulse is detected by detector 169-A through deflector 169-C as shown in FIG. 173. The time taken by the pulse to be reflected back from the retina is measured by the detection system, e.g., reflector 169-C, detector 169-A, and computer 1. This measurement is used to provide an axial length and the pupil diameter. The alignment is important in determining the cylinder axis, therefore, orienting the patient with the device horizontally is very important.

In an exemplary embodiment, only one laser device is used. In this exemplary embodiment, laser 15 and laser 169-D are the same device.

In an exemplary embodiment, only one detector device is used. In this exemplary embodiment, detector 13 and detector 169-A are the same device. In other words, in this embodiment, there is one detector that detects both a scanning laser and also detects based on the time of flight principle.

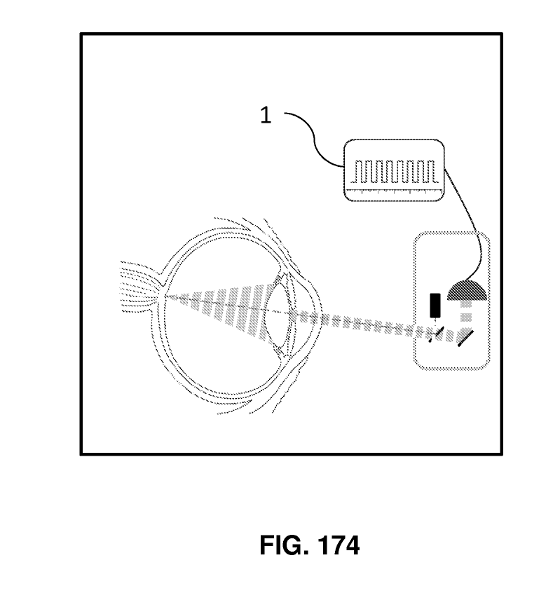

As stated above, the Autorefractor-Keratometer of the present disclosure provides a 3D digital surface, determines an ocular axial length along the optical path using a laser rangefinder, and determines pupil diameter. The reflection of the laser pulse from the retina is shown in FIG. 171. As shown in FIGS. 172 to 173, light is reflected through the pupil and onto reflector 169-C. The width of that reflected light allows the detector to determine the pupil diameter measurement. From the pupil diameter measurement, sphero-cylinder power for day and night time environments can be determined by appropriate calculation. Time of flight and width pupil diameter information is collected by computer 1 as shown in FIG. 174.

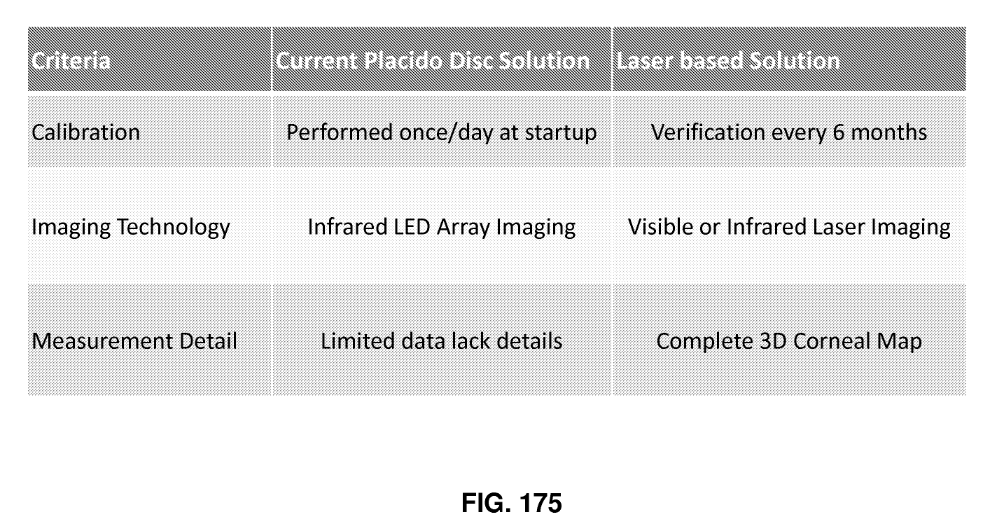

FIG. 175 shows a direct comparison of calibration, imaging technology, and measurement detail from the prior art Autorefractor-Keratometers. As before, calibration times change from once each day to simple verification twice a year. The imaging technology is limited to a particular array instead of using laser imaging. Finally, a complete 3D map of the cornea is provided instead of the limited data in the prior art devices. Thus, each characteristic is improved over the prior art.

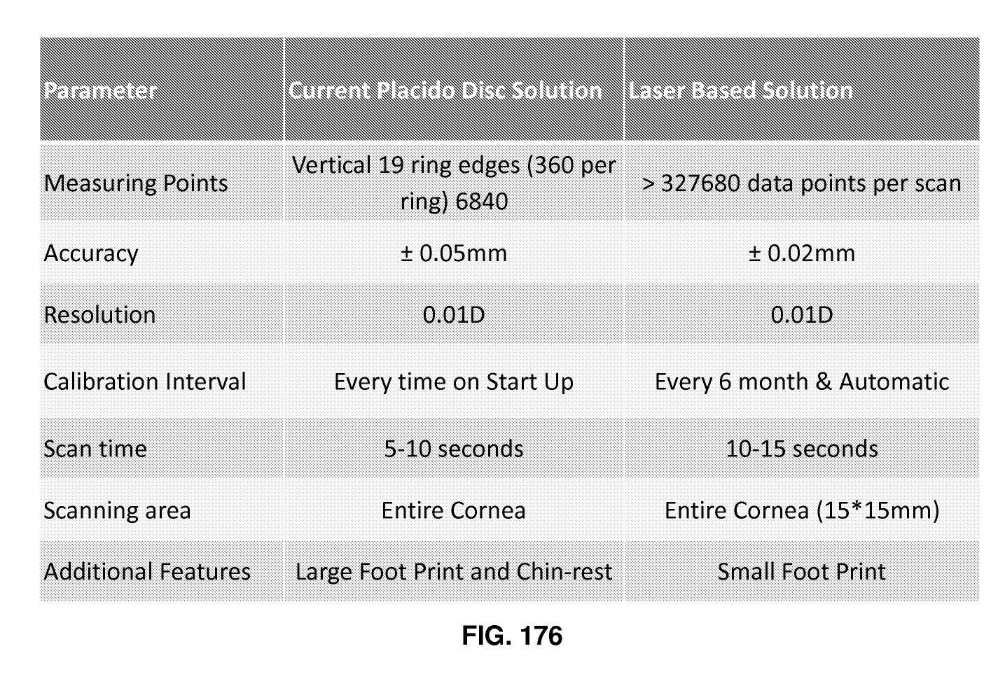

FIG. 176 shows a direct comparison of the specifications of prior art Placido disc solutions with an exemplary embodiment of a 3D laser scanning system described herein. First, the number of measuring points and resolution of the Placido disc solution is comparable with the present laser-based solution. However, the accuracy of the present devices and methods is significantly improved by a factor of 2.5 over the prior art. Calibration is vastly superior, changing from daily to bi-annual verification. As for scanning time and scanning area, these parameters are similar for both solutions. Finally, the laser-based solution of the present disclosure, however, requires a much smaller footprint.

Advantages of the present Autorefractor-Keratometer are numerous. The present Autorefractor-Keratometer is more accurate and provides more complete and high resolution data. Little or no user interaction is needed for scanning or calibration. The present Autorefractor-Keratometer provides an accurate and precise prescription power determination. The present Autorefractor-Keratometer provides an accurate and precise corneal topography. Data from the Autorefractor-Keratometer can be shared across the cloud, for example, as shown in FIGS. 126, 127, and 128. In addition, the present Autorefractor-Keratometer provides significant cost savings over prior art Autorefractor-Keratometers and is easy to use.

FIGS. 177 to 179 illustrate a glasses frame, e.g., an eyeglass frame, mounted in a frame holder inside the 3D laser scanner and shows, with a transparent cone and cylinder, a field of view of the camera that is large enough to engulf the entire depth of the eyeglass frame while it is being scanned by a laser line from the laser. The camera and laser are moveable by the linear drive mechanism. The embodiment of FIGS. 177 to 179 differs from the embodiment shown in FIGS. 38D to 38H in that in this embodiment, the camera is orthogonal and the laser is placed at an angle. The advantage of the new orientation is that the laser light scattering that occurred from the nasal and temple regions of the frame with the previous laser-camera orientation now occur at the top and/or bottom eye wire of the frame. This reduced laser scattering reduces the total affected frame area. This also improves the edge detection in this area and the frame edge is correctly determined. Alternately, the data from the laser scattering area may be ignored and new data may be interpolated to create the frame trace.

It is noted that various individual features of the inventive processes and systems may be described only in one exemplary embodiment herein. The particular choice for description herein with regard to a single exemplary embodiment is not to be taken as a limitation that the particular feature is only applicable to the embodiment in which it is described. All features described herein are equally applicable to, additive, or interchangeable with any or all of the other exemplary embodiments described herein and in any combination or grouping or arrangement. In particular, use of a single reference numeral herein to illustrate, define, or describe a particular feature does not mean that the feature cannot be associated or equated to another feature in another drawing figure or description. Further, where two or more reference numerals are used in the figures or in the drawings, this should not be construed as being limited to only those embodiments or features, they are equally applicable to similar features or not a reference numeral is used or another reference numeral is omitted.

The phrase “at least one of A and B” is used herein and/or in the following claims, where A and B are variables indicating a particular object or attribute. When used, this phrase is intended to and is hereby defined as a choice of A or B or both A and B, which is similar to the phrase “and/or”. Where more than two variables are present in such a phrase, this phrase is hereby defined as including only one of the variables, any one of the variables, any combination of any of the variables, and all of the variables.

The foregoing description and accompanying drawings illustrate the principles, exemplary embodiments, and modes of operation of the invention. However, the invention should not be construed as being limited to the particular embodiments discussed above. Additional variations of the embodiments discussed above will be appreciated by those skilled in the art and the above-described embodiments should be regarded as illustrative rather than restrictive. Accordingly, it should be appreciated that variations to those embodiments can be made by those skilled in the art without departing from the scope of the invention as defined by the following claims.

1. An autorefractor-keratometer device, comprising:
a first laser scanning device that emits a plurality of laser line scans;
a second laser device that emits a laser pulse;
a first detector that detects the plurality of emitted laser line scans to determine corneal topography; and
a second detector that detects a reflection of the emitted laser pulse to determine sphero-cylinder refraction.

2. The autorefractor-keratometer device of claim 1, wherein the first laser is configured to be orthogonal to a cornea and the first detector is configured to be at an angle to the cornea.

3. The autorefractor-keratometer device of claim 1, wherein the first detector is configured to be orthogonal to a cornea and the first laser is configured to be at an angle to the cornea.

4. The autorefractor-keratometer device of claim 1, wherein the autorefractor-keratometer device is configured within a handheld device.

5. The autorefractor-keratometer device of claim 1, wherein corneal topography is determined by scanning from a top eyelid to a bottom of an eye.

6. The autorefractor-keratometer device of claim 1, wherein corneal topography is determined by scanning from a bottom of an eye to a top eyelid.

7. The autorefractor-keratometer device of claim 1, wherein corneal topography is determined by scanning from a temporal side of an eye to a nasal side of the eye.

8. The autorefractor-keratometer device of claim 1, wherein corneal topography is determined by scanning from a nasal side of an eye to a temporal side of the eye.

9. The autorefractor-keratometer device of claim 1, wherein the first laser scanning device comprises a 3-dimensional laser scanning device.

10. The autorefractor-keratometer device of claim 9, wherein the first detector comprises a CMOS camera detector.

11. The autorefractor-keratometer device of claim 10, wherein the second detector comprises a detector that detects based on a time of flight principle.

12. The autorefractor-keratometer device of claim 11, wherein the second laser device comprises an infrared laser diode.

13. The autorefractor-keratometer device of claim 1, wherein a single laser device emits the plurality of laser line scans and the laser pulse.

14. The autorefractor-keratometer device of claim 1, wherein a single detector both detects laser line scans and detects on a time of flight principle.

15. The autorefractor-keratometer device of claim 1, wherein:
a single laser device emits the plurality of laser line scans and the laser pulse; and
a single detector both detects laser line scans and detects on a time of flight principle.

16. The autorefractor-keratometer device of claim 1, wherein the first laser scanning device provides at least 327,680 data points per scan.

17. The autorefractor-keratometer device of claim 1, wherein an accuracy of the first laser scanning device is ±0.02 mm.

18. The autorefractor-keratometer device of claim 1, wherein a resolution of the first laser scanning device is 0.01 Diopters.
